SEQUENCE LISTING (47 sequences)

SEQ ID NO: 1 - protein, 9 aa, synthetic construct
VMAPRTLVL

SEQ ID NO: 2 - protein, 9 aa, synthetic construct
VMAPRTVLL

SEQ ID NO: 3 - protein, 9 aa, synthetic construct
VMAPRTLIL

SEQ ID NO: 4 - protein, 9 aa, synthetic construct
VMAPRTLFL

SEQ ID NO: 5 - protein, 9 aa, synthetic construct
QMRPVSRVL

SEQ ID NO: 6 - protein, 9 aa, synthetic construct
VMAPRTLLL

SEQ ID NO: 7 - protein, 9 aa, synthetic construct
VMAPRSLLL

SEQ ID NO: 8 - protein, 9 aa, synthetic construct
VMAPRILIL

SEQ ID NO: 9 - protein, 9 aa, synthetic construct
RMYSPTSIL

SEQ ID NO: 10 - protein, 9 aa, synthetic construct
RLPAKAPLL

SEQ ID NO: 11 - DNA, 38 bp, synthetic construct
  source 1..38: mol_type = other DNA
taatacgactcactatagcagctcccggaggtgcaaaa

SEQ ID NO: 12 - DNA, 35 bp, synthetic construct
  source 1..35: mol_type = other DNA
ttctagctctaaaacttttgcacctccgggagctg

SEQ ID NO: 13 - protein, 8 aa, synthetic construct
VMAPRTLL

SEQ ID NO: 14 - DNA, 27 bp, synthetic construct
  source 1..27: mol_type = other DNA
gtgatggccccaagaaccctgatcctg

SEQ ID NO: 15 - DNA, 27 bp, synthetic construct
  source 1..27: mol_type = other DNA
gtgatggccccccggaccctggtgctg

SEQ ID NO: 16 - DNA, 27 bp, synthetic construct
  source 1..27: mol_type = other DNA
gtcatggcgccccgaaccgtcctcctg

SEQ ID NO: 17 - DNA, 27 bp, synthetic construct
  source 1..27: mol_type = other DNA
gtgatggccccaagaaccctgttcctg

SEQ ID NO: 18 - DNA, 27 bp, synthetic construct
  source 1..27: mol_type = other DNA
cagatgagaccggtgtccagggtactg

SEQ ID NO: 19 - DNA, 27 bp, synthetic construct
  source 1..27: mol_type = other DNA
gtgatggccccccggaccctgctgctg

SEQ ID NO: 20 - DNA, 27 bp, synthetic construct
  source 1..27: mol_type = other DNA
gtgatggccccccggagcctgctgctg

SEQ ID NO: 21 - DNA, 27 bp, synthetic construct
  source 1..27: mol_type = other DNA
gtgatggccccccggatcctgatcctg

SEQ ID NO: 22 - DNA, 27 bp, synthetic construct
  source 1..27: mol_type = other DNA
cggatgtacagccccaccagcatcctg

SEQ ID NO: 23 - DNA, 27 bp, synthetic construct
  source 1..27: mol_type = other DNA
cggctgcccgccaaggcccccctgctg

SEQ ID NO: 24 - protein, 23 aa, synthetic construct
GTGATGGCCCCCCGGACCCTGMT

SEQ ID NO: 25 - protein, 20 aa, synthetic construct
MSRSVALAVLALLSLSGLEA

SEQ ID NO: 26 - DNA, 60 bp, synthetic construct
  source 1..60: mol_type = other DNA
atgtcacgctctgtcgctcttgcagtacttgccctgttgagcctcagcggactcgaagcc

SEQ ID NO: 27 - DNA, 60 bp, synthetic construct
  source 1..60: mol_type = other DNA
tacagtgcgagacagcgagaacgtcatgaacgggacaactcggagtcgcctgagcttcgg

SEQ ID NO: 28 - protein, 15 aa, synthetic construct
GGGASGGGGSGGGGS

SEQ ID NO: 29 - DNA, 45 bp, synthetic construct
  source 1..45: mol_type = other DNA
ggaggaggtgcgagcggtggtggaggtagcggaggtggaggaagc

SEQ ID NO: 30 - DNA, 45 bp, synthetic construct
  source 1..45: mol_type = other DNA
cctcctccacgctcgccaccacctccatcgcctccacctccttcg

SEQ ID NO: 31 - protein, 99 aa, synthetic construct [sequence omitted]

SEQ ID NO: 32 - DNA, 297 bp, synthetic construct
  source 1..297: mol_type = other DNA
attcaaaggacaccgaaaatccaagtatatagcaggcaccccgctgaaaacggcaaaagcaactttctcaactgttacgtctccggcttccaccccagtgatatcgaggtcgatctgctcaagaacggcgaacgcatcgagaaggttgaacacagtgatctgtcatttagtaaagattggtcattttaccttctctattatacagagtttacaccaacggagaaggacgaatacgcatgtcgcgttaatcacgtcacgctttctcaacctaaaattgtgaaatgggaccgtgatatg

SEQ ID NO: 33 - DNA, 297 bp, synthetic construct
  source 1..297: mol_type = other DNA
taagtttcctgtggcttttaggttcatatatcgtccgtggggcgacttttgccgttttcgttgaaagagttgacaatgcagaggccgaaggtggggtcactatagctccagctagacgagttcttgccgcttgcgtagctcttccaacttgtgtcactagacagtaaatcatttctaaccagtaaaatggaagagataatatgtctcaaatgtggttgcctcttcctgcttatgcgtacagcgcaattagtgcagtgcgaaagagttggattttaacactttaccctggcactatac

SEQ ID NO: 34 - protein, 20 aa, synthetic construct
GGGGSGGGGSGGGGSGGGGS

SEQ ID NO: 35 - DNA, 60 bp, synthetic construct
  source 1..60: mol_type = other DNA
ggaggcggtgggtcaggtggaggtgggtctggcggaggtggatccggtggtggaggtagt

SEQ ID NO: 36 - DNA, 60 bp, synthetic construct
  source 1..60: mol_type = other DNA
cctccgccacccagtccacctccacccagaccgcctccacctaggccaccacctccatca

SEQ ID NO: 37 - protein, 337 aa, synthetic construct
GSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 38 - DNA, 1014 bp, synthetic construct
  source 1..1014: mol_type = other DNA
ggctcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttgtaa

SEQ ID NO: 39 - DNA, 1014 bp, synthetic construct
  source 1..1014: mol_type = other DNA
ccgagggtgaggaacttcataaaggtgtgaaggcacagggccgggccggcgcccctcggggcgaagtagagacacccgatgcacctgctgtgggtcaagcacgcgaagctgttgctgcggcgctcaggctcctaccacggcgcccgcggcacctacctcgtcctccccagtctcataaccctggccctctgtgcctcgcggtccctgtggcgtgtctaaaaggctcacttagacgcctgcgacgcgccgatgatgttagtctcgctccggcccagagtgtgggacgtcacctacgtaccgacgctcgaccccgggctgcccgcgaaggaggcgcccatacttgtcaagcggatgctgccgttcctaatagagtgggacttactcctggacgcgaggacctggcgccacctgtgccgccgagtctagaggctcgttttcagtttactacggagactccgcctcgtggtctctcggatggaccttctgtgtacgcacctcaccgaggtgtttatggacctcttccccttcctctgcgacgaagtggacctcgggggtttctgtgtgcactgagtggtggggtagagactggtactccggtgggactccacgacccgggacccgaagatgggacgcctctagtgtgactggaccgtcgtcctacccctcccggtatgggtcctgtgcctcgagcacctctggtccggacgtcccctaccttggaaggtcttcacccgtcgacaccaccacggaagacctctcctcgtctctatgtgcacggtacacgtcgtactccccgatgggctcgggcagtgggactctaccttcggccgaagggtcgggtggtaggggtagcacccgtagtaacgaccggaccaagaggaacctagacaccagagacctcgacaccaacgacgacactatacctccttcttctcgagtccaccttttcctccctcgatgagattccgactcacctcgctgtcacgggtccccagactcagagtgtcgaacatt

SEQ ID NO: 40 - protein, 500 aa, synthetic construct
MSRSVALAVLALLSLSGLEAVMAPRTLILGGGASGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 41 - DNA, 1521 bp, synthetic construct
  source 1..1521: mol_type = other DNA
tctagagccaccatgtcacgctctgtcgctcttgcagtacttgccctgttgagcctcagcggactcgaagccgtgatggccccaagaaccctgatcctgggaggaggtgcgagcggtggtggaggtagcggaggtggaggaagcattcaaaggacaccgaaaatccaagtatatagcaggcaccccgctgaaaacggcaaaagcaactttctcaactgttacgtctccggcttccaccccagtgatatcgaggtcgatctgctcaagaacggcgaacgcatcgagaaggttgaacacagtgatctgtcatttagtaaagattggtcattttaccttctctattatacagagtttacaccaacggagaaggacgaatacgcatgtcgcgttaatcacgtcacgctttctcaacctaaaattgtgaaatgggaccgtgatatgggaggcggtgggtcaggtggaggtgggtctggcggaggtggatccggtggtggaggtagtggctcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttgtaagtcgac

SEQ ID NO: 42 - DNA, 1521 bp, synthetic construct
  source 1..1521: mol_type = other DNA
agatctcggtggtacagtgcgagacagcgagaacgtcatgaacgggacaactcggagtcgcctgagcttcggcactaccggggttcttgggactaggaccctcctccacgctcgccaccacctccatcgcctccacctccttcgtaagtttcctgtggcttttaggttcatatatcgtccgtggggcgacttttgccgttttcgttgaaagagttgacaatgcagaggccgaaggtggggtcactatagctccagctagacgagttcttgccgcttgcgtagctcttccaacttgtgtcactagacagtaaatcatttctaaccagtaaaatggaagagataatatgtctcaaatgtggttgcctcttcctgcttatgcgtacagcgcaattagtgcagtgcgaaagagttggattttaacactttaccctggcactataccctccgccacccagtccacctccacccagaccgcctccacctaggccaccacctccatcaccgagggtgaggaacttcataaaggtgtgaaggcacagggccgggccggcgcccctcggggcgaagtagagacacccgatgcacctgctgtgggtcaagcacgcgaagctgttgctgcggcgctcaggctcctaccacggcgcccgcggcacctacctcgtcctccccagtctcataaccctggccctctgtgcctcgcggtccctgtggcgtgtctaaaaggctcacttagacgcctgcgacgcgccgatgatgttagtctcgctccggcccagagtgtgggacgtcacctacgtaccgacgctcgaccccgggctgcccgcgaaggaggcgcccatacttgtcaagcggatgctgccgttcctaatagagtgggacttactcctggacgcgaggacctggcgccacctgtgccgccgagtctagaggctcgttttcagtttactacggagactccgcctcgtggtctctcggatggaccttctgtgtacgcacctcaccgaggtgtttatggacctcttccccttcctctgcgacgaagtggacctcgggggtttctgtgtgcactgagtggtggggtagagactggtactccggtgggactccacgacccgggacccgaagatgggacgcctctagtgtgactggaccgtcgtcctacccctcccggtatgggtcctgtgcctcgagcacctctggtccggacgtcccctaccttggaaggtcttcacccgtcgacaccaccacggaagacctctcctcgtctctatgtgcacggtacacgtcgtactccccgatgggctcgggcagtgggactctaccttcggccgaagggtcgggtggtaggggtagcacccgtagtaacgaccggaccaagaggaacctagacaccagagacctcgacaccaacgacgacactatacctccttcttctcgagtccaccttttcctccctcgatgagattccgactcacctcgctgtcacgggtccccagactcagagtgtcgaacattcagctg

SEQ ID NO: 43 - DNA, 27 bp, synthetic construct
  source 1..27: mol_type = other DNA
cactaccggggttcttgggactaggac

SEQ ID NO: 44 - protein, 337 aa, synthetic construct
GSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDRRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 45 - DNA, 1011 bp, synthetic construct
  source 1..1011: mol_type = other DNA
ggctcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaacctgcggacgctgcgcggctactacaatcagagcgaggccgggtctcacaccctgcagtggatgcatggctgcgagctggggcccgacaggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttg

SEQ ID NO: 46 - protein, 9 aa, synthetic construct
RIIPRHLQL

SEQ ID NO: 47 - DNA, 27 bp, synthetic construct
  source 1..27: mol_type = other DNA
cggatcatcccccggcacctgcagctg

(19) United States
(12) Patent Application Publication
(10) Pub. No.: US 2026/0042815 A1
(43) Pub. Date: Feb. 12, 2026

(54) UNIVERSAL T CELLS AND COMPOSITIONS AND METHODS OF USE THEREOF

(71) Applicant: The Trustees of the University of Pennsylvania, Philadelphia, PA (US)

(72) Inventors: Gerald P. Linette, Gladwyne, PA (US); Beatriz Carreno, Gladwyne, PA (US); Chong Xu, Mount Laurel, NJ (US); Kimberly Apodaca, Philadelphia, PA (US)

(21) Appl. No.: 19/102,972

(22) PCT Filed: Aug. 11, 2023
(86) PCT No.: PCT/US2023/072072

§ 371 Date: Feb. 11, 2025

Related U.S. Application Data
(60) Provisional application No. 63/371,144, filed on Aug. 11, 2022.
(60) Provisional application No. 63/382,766, filed on Nov. 8, 2022.

(51) Int. Cl. C07K 14/74; A61K 40/11; A61K 40/31; A61P 35/00; C12N 5/0783

(52) U.S. Cl. CPC C07K 14/70539; A61K 40/11; A61K 40/31; A61P 35/00; C12N 5/0636; C07K 2319/02; C12N 2510/00

(57) ABSTRACT

The invention provides a modified HLA-E single chain trimer construct and nucleic acid molecules encoding thereof as well as cells and compositions comprising thereof for increasing the persistence or reducing the clearance of at least one cell of interest. In some embodiments, the present invention also provides methods of preventing and/or eliminating alloresponse, allorecognition, and/or allogeneic rejection. The invention also relates to chimeric antigen receptor (CAR) cells or engineered TCR-expressing T cells comprising the modified HLA-E single chain trimer construct and/or the nucleic acid molecules encoding thereof.

### CROSS-REFERENCE TO RELATED APPLICATIONS

This application claims priority to U.S. Provisional Application Ser. No. 63/371,144, filed Aug. 11, 2022, and U.S. Provisional Application Ser. No. 63/382,766, filed Nov. 8, 2022, the disclosures of which are hereby incorporated by reference herein in their entireties.

### STATEMENT REGARDING FEDERALLY SPONSORED RESEARCH OR DEVELOPMENT

This invention was made with government support under CA244711 awarded by National Institutes of Health. The government has certain rights in the invention.

### BACKGROUND OF THE INVENTION

Chimeric antigen receptor (CAR) T cell therapy is a form of T cell transfer therapy in which T cells are harvested from the patient and modified to express antigen receptors that are not typically expressed (Ahmad, A., 2020, International Journal of Molecular Sciences, 21(12):4303). Clinical development of CAR T cell products, however, is often hampered by low yield and poor functionality of peripheral blood T cells from cancer patients, especially individuals that received prior systemic therapy. Patients have dysfunctional T cells due to tumor microenvironment and pre-treatments (Thommen et al., Cancer Cell Vol. 33:547-562, 2018) and there is a delay in treatment time (Levine et al., Mol Ther Methods Clin Dev Vol. 4:92-101, 2017). Additional disadvantages of current CAR T cells is that CAR T cells are sensitive to host natural killer (NK) cell recognition due to lack of major histocompatibility complex class I molecule (MHC I) and the risk of graft vs host disease (GvHD).

Thus, there is a need in the art for a “universal” T cell product that can be engineered to optimize persistence and avoid rejection by the host's immune system. The present invention meets this unmet need.

### SUMMARY OF THE INVENTION

In various aspects, the present invention provides a peptide comprising: a human leukocyte antigen (HLA) signal peptide or a fragment thereof comprising at least one amino acid sequence that is at least about 70% identical to the amino acid sequence selected from SEQ ID NOs: 1-10, 13, and 46, a modified beta-2-microglobulin (B2M) or a fragment thereof, and an HLA class I histocompatibility antigen, alpha chain E (HLA-E) or a fragment thereof. In some embodiments, the peptide is a single chain trimer.

In some embodiments, the HLA signal peptide comprises at least one selected from HLA-A*02:01 signal peptide or a fragment thereof, HLA-B*08:01 signal peptide or a fragment thereof, HLA-C*03:01 signal peptide or a fragment thereof, HLA-G signal peptide or a fragment thereof, HSP60 signal peptide or a fragment thereof, CMV Towne signal peptide or a fragment thereof, CMV AF1 signal peptide or a fragment thereof, CMV 109b signal peptide or a fragment thereof, RL9HIV signal peptide or a fragment thereof, or Mtb44 signal peptide or a fragment thereof.

In some embodiments, the HLA-E is a human HLA-E or a fragment thereof.

In some embodiments, the modified B2M comprises at least one selected from a modified B2M signal peptide or a fragment thereof or leader-less modified B2M or a fragment thereof. In some embodiments, the modified B2M signal peptide or the fragment thereof is linked to the HLA signal peptide or the fragment thereof, the HLA signal peptide or the fragment is linked to the leader-less modified B2M or the fragment thereof, and the leader-less modified B2M or the fragment thereof is linked to the HLA-E or the fragment thereof.

In some embodiments, the peptide further comprises at least one linker and at least one spacer.

In some embodiments, the peptide comprises an amino acid sequence that is at least about 70% identical to the amino acid sequence set forth in SEQ ID NO: 40.

In some embodiments, the peptide prevents, reduces, or inhibits a natural killer (NK) cell-mediated killing of at least one donor cell. In some embodiments, the peptide increases the persistence or reduces the clearance of at least one donor cell.

In various aspects, the present invention provides to a composition comprising at least one peptide of the present invention. In some embodiments, the composition is a pharmaceutically acceptable composition.

In various aspects, the present invention also provides a nucleic acid molecule comprising: a nucleotide sequence encoding a HLA signal peptide or a fragment thereof that comprises at least one amino acid sequence that is at least about 70% identical to the amino acid sequence selected from SEQ ID NOs: 1-10, 13, and 46, a nucleotide sequence encoding a modified B2M or a fragment thereof, and a nucleotide sequence encoding an HLA-E or a fragment thereof.

In some embodiments, the nucleotide sequence encoding the HLA signal peptide or the fragment thereof comprises at least one nucleotide sequence that is at least about 70% identical to the nucleotide sequence selected from SEQ ID NOs: 14-24 and 47.

In some embodiments, the nucleic acid molecule comprises a nucleotide sequence that is at least about 70% identical to the nucleotide sequence set forth in SEQ ID NO: 41.

In other aspects, the present invention also provides a nucleic acid molecule comprising a nucleotide sequence encoding a HLA signal peptide or a fragment thereof comprising at least one nucleotide sequence that is at least about 70% identical to the nucleotide sequence selected from SEQ ID NOs: 14-24 and 47, a nucleotide sequence encoding a modified B2M or a fragment thereof, and a nucleotide sequence encoding an HLA-E or a fragment thereof;

In some embodiments, the nucleotide sequence encoding the modified B2M comprises at least one selected from the group consisting of a nucleotide sequence encoding a modified B2M signal peptide or a fragment thereof and a nucleotide sequence encoding a leader-less modified B2M or a fragment thereof.

In some embodiments, the nucleotide sequence encoding the modified B2M signal peptide or the fragment thereof is linked to the nucleotide sequence encoding the HLA signal peptide or the fragment thereof; the nucleotide sequence encoding the HLA signal peptide or the fragment is linked to the nucleotide sequence encoding the leader-less modified B2M or the fragment thereof; and the nucleotide sequence encoding the leader-less modified B2M or the fragment thereof is linked to the nucleotide sequence encoding the HLA-E or the fragment thereof.

In some embodiments, the nucleic acid molecule further comprises at least one nucleotide sequence encoding a linker and at least one nucleotide sequence encoding a spacer.

In some embodiments, the nucleic acid molecule comprises a nucleotide sequence that is at least about 70% identical to the nucleotide sequence set forth in SEQ ID NO: 41.

In some embodiments, the nucleic acid molecule prevents, reduces, or inhibits a NK cell-mediated killing of at least one donor cell. In some embodiments, the nucleic acid molecule increases the persistence or reduces the clearance of at least one donor cell.

In various aspects, the present invention also provides a composition comprising at least one nucleic acid molecule of the present invention.

In other aspects, the present invention provides a genetically engineered cell comprising at least one nucleic acid molecule of the present invention.

In some embodiments, the genetically engineered cell is modified to not express at least one selected from a B2M, class II major histocompatibility complex transactivator (CIITA), or native T cell receptor (TCR).

In some embodiments, the genetically engineered cell is a triple knockout (TKO) cell that does not express at least one selected from a major histocompatibility complex (MHC) I, MHC II, or native TCR.

In some embodiments, the genetically engineered cell is selected from an autologous cell, allogenic cell, alloresponsive cell, T cell, induced pluripotent stem cell (IPSC), chimeric antigen receptor (CAR) cell, engineered TCR cell, or any combination thereof.

In some embodiments, the T cell is selected from an alloresponsive T cell, T cell bearing engineered TCRs, allo-specific T cell, T cell bearing alloreactive TCR, CAR T cell, engineered TCR-expressing T cell, or any combination thereof.

In various aspects, the present invention provides a method of preventing, reducing, or eliminating an allograft or xenograft rejection in a subject in need thereof, the method comprising administering a therapeutically effective amount of at least one cell of the present invention to the subject.

In various aspects, the present invention provides a method of preventing, reducing, or eliminating an alloresponse in a subject in need thereof, the method comprising administering a therapeutically effective amount of at least one cell of the present invention to the subject.

In various aspects, the present invention provides a method of depleting the level of an alloresponsive cell in a subject in need thereof, the method comprising administering a therapeutically effective amount of at least one cell of the present invention to the subject.

In various aspects, the present invention provides a method of inducing an allogeneic tolerance in a subject in need thereof, the method comprising administering a therapeutically effective amount of at least one cell of the present invention to the subject.

In various aspects, the present invention provides a method of improving the effectiveness of a CAR cell therapy in a subject in need thereof, the method comprising administering a therapeutically effective amount of at least one CAR cell of the present invention to the subject.

In various aspects, the present invention provides a method of improving the effectiveness of an engineered TCR cell therapy in a subject in need thereof, the method comprising administering a therapeutically effective amount of at least one engineered TCR cell of the present invention to the subject.

In some embodiments, the subject has at least one selected from an organ transplantation, tissue transplantation, cell transplantation, allotransplantation, intestinal transplantation, reconstructive transplantation, autoimmune disease or disorder, graft-versus-host disease (GvHD), disease or disorder associated with at least one HLA receptor, disease or disorder associated with at least one HLA-containing receptor, disease or disorder associated with at least one MHC receptor, disease or disorder associated with at least one MHC-containing receptor, disease or disorder associated with expression of alloresponsive cells, disease or disorder associated with organ transplantation, disease or disorder associated with tissue transplantation, disease or disorder associated with cell transplantation, disease or disorder associated with allotransplantation, disease or disorder associated with intestinal transplantation, cancer, disease or disorder associated with cancer, or disease or disorder associated with reconstructive transplantation.

In various aspects, the present invention provides a method of preventing or treating a disease or disorder in a subject in need thereof, the method comprising administering a therapeutically effective amount of at least one cell of the present invention to the subject. In some embodiments, the method comprises depleting, reducing, or eliminating the level of at least one selected from an alloresponsive cell, immune cell, T cell, B cell, NK cell, white blood cell, myeloid cell, or plasma cell.

In some embodiments, the method is an engineered TCR cell therapy.

In some embodiments, the disease or disorder is selected from a disease or disorder associated with expression of alloresponsive cells, disease or disorder associated with at least one HLA receptor, disease or disorder associated with at least one HLA-containing receptor, disease or disorder associated with at least one MHC receptor, disease or disorder associated with at least one MHC-containing receptor, GvHD, autoimmune disease or disorder, disease or disorder associated with organ transplantation, disease or disorder associated with tissue transplantation, disease or disorder associated with cell transplantation, disease or disorder associated with allotransplantation, disease or disorder associated with intestinal transplantation, disease or disorder associated with reconstructive transplantation, cancer, disease or disorder associated with cancer, or any combination thereof.

In some embodiments, the disease or disorder is selected from the group consisting of a cancer, disease or disorder associated with cancer, and any combination thereof.

In some embodiments, the disease or disorder associated with expression of alloresponsive cell is selected from an allograft rejection, immune rejection, chronic allogeneic rejection, engraftment rejection, transplant rejection, inflammation, inflammation caused by ischemia/reperfusion, infection, immune response to an allograft, or any combination thereof.

In some embodiments, the subject had at least one selected from an organ transplantation, tissue transplantation, cell transplantation, allotransplantation, intestinal transplantation, or reconstructive transplantation.

In various aspects, the present invention provides a method of preventing or treating a cancer, disease or disorder associated with cancer, or a combination thereof in a subject in need thereof, the method comprising administering a therapeutically effective amount of at least one cell of the present invention.

In some embodiments, the method is an engineered TCR cell therapy.

### BRIEF DESCRIPTION OF THE DRAWINGS

The following detailed description of exemplary embodiments of the invention will be better understood when read in conjunction with the appended drawings. For the purpose of illustrating the invention, there are shown in the drawings exemplary embodiments. It should be understood, however, that the invention is not limited to the precise arrangements and instrumentalities of the embodiments shown in the drawings.

FIG. 1 depicts a schematic representation of universal CAR T (UCART) cells that overcome disadvantages of current CAR T cell manufacturing process.

FIG. 2 depicts schematic representations of normal conditions, knockout (KO) of MHC I induced NK cell killing due to “missing self” of NK cells (Karre et la., Immunol Today Vol. 11(7), 1990); and NK cells recognized stress ligands, which upregulated on surface of T cells.

FIG. 3 depicts a schematic representation of preventing the clearance of UCART cells through expression and interaction of human leukocyte antigen (HLA) class I histocompatibility antigen, alpha chain E (HLA-E) with cluster of differentiation 159 (CD159) NK receptor (NKG2A).

FIG. 4 depicts a schematic representation of designing the optimal HLA-E single chain trimer (SCT).

FIG. 5 depicts a schematic representation of mitigating lysis of target cells due to NKG2C/HLA-E interaction.

FIG. 6 depicts representative results demonstrating transducing K562 with HLA-E single chain dimer (SCD) and SCT.

FIG. 7 depicts representative generation of stress-ligand single KOs on K562 cells, such as MHC class I polypeptide-related sequence A (MICA) KO.

FIG. 8 depicts representative results demonstrating that single KOs of NK cell ligands on HLA-E+ K562 cells did not reduce residual lysis in ⁵¹Cr release assays.

FIG. 9 depicts representative results demonstrating that blocking DNAM-1 or 2B4 did not reduce NK cell activation.

FIG. 10 depicts representative results demonstrating that HLA-C*03:01 leader peptide was the optimal peptide to inhibit NK cells from cytomegalovirus (CMV)- and CMV+ donors.

FIG. 11 depicts representative results demonstrating that HLA-E SCT presenting HLA-C leader peptide results in the most significant NK cell inhibition.

FIG. 12 depicts representative results demonstrating that HLA-E+ SCT conferred protection from NK cell-mediated killing.

FIG. 13 depicts representative characterization of TKO HLA-E+ T cells. Phenotype shown at day 14 of T cell expansion. Bulk T cells activated with anti-CD3/28 beads for 5 days before EP at 3:1 bead:cell ratio.

FIG. 14 depicts representative results demonstrating that expression of HLA-E+ on TKO T cells inhibited the “missing-self” response of NK cells and that the absence of MHC class I on TKO cells resulted in activation of NK cells due to “missing-self” response.

FIG. 15 depicts representative results demonstrating that HLA-E+ SCT consistently reduced cell lysis mediated by NK cells from different donors.

FIG. 16 depicts representative results demonstrating that activated NK cells are distinguished by CD69, CD25, and CD8 upregulation.

FIG. 17 depicts representative results demonstrating phenotype of activated vs resting NK cells.

FIG. 18 depicts representative results demonstrating that NKG2A/C expression increased with NK cell activation.

FIG. 19 depicts representative results demonstrating that inhibition of NK cells when co-cultured with HLA-E SCT+ K562 target cells.

FIG. 20 depicts representative results demonstrating that blocking stress ligands on K562 did not significantly reduce activation of NK cells.

FIG. 21 depicts representative results demonstrating that HLA-E+ SCT inhibited both resting and activated NK cells.

FIG. 22 depicts a schematic representation that NK cell deficiency is associated with active human CMV (HCMV) infection.

FIG. 23 depicts a schematic representation of expression of human NK cells and separation of NKG2A+ and NKG2C+ subsets.

FIG. 24 depicts representative results demonstrating that NKG2A+ NK cells, but not NKG2C+ NK cells, are inhibited by HLA-E+ K562 cells.

FIG. 25 depicts a schematic representation of generation of TKO HLA-E+ SCT cells.

FIG. 26 depicts representative results demonstrating that TKO T cells were not recognized by unrelated donor T cells as there is no measurable alloreactivity.

FIG. 27 depicts representative HLA signal peptide alignments.

FIG. 28 depicts representative nucleotide sequence encoding HLA signal peptide alignments.

FIG. 29 depicts representative HLA-C signal peptide-HLA-E SCT sequences.

FIG. 30 depicts representative results of a 4-hour ⁵¹Cr release assay demonstrating HLA-E presenting HLA-C*03:01 leader peptide resulted in the most significant reduction in K562 lysis indicating its ability to inhibit NK cell activation due to the “missing-self” response. Shown at 2:1 E:T ratio.

FIG. 31 depicts representative results demonstrating HLA-E+ K562 cells inhibited NK cells from different donors and effect depended on phenotypic differences of donor NK cell populations.

FIG. 32 depicts representative results demonstrating surface expression of NKG2A and NKG2C of donors shown in FIG. 31.

FIG. 33 depicts representative results demonstrating NKG2A and NKG2C surface expression of expanded NK cells form 16 different normal donors.

FIG. 34, comprising FIG. 34A and FIG. 34B, depicts representative results demonstrating sorted NKG2A+ NK cells were inhibited by HLA-E+ K562 cells and sorted NKG2C+ NK cells were not inhibited by HLA-E expression on K562. (**P<0.0025, ***P<0.000125; data shown at 2:1 E:T ratio) FIG. 34A depicts representative results demonstrating sorted NKG2A+ NK cells were inhibited by HLA-E+ K562 cells. FIG. 34B depicts representative results demonstrating sorted NKG2C+ NK cells were not inhibited by HLA-E expression on K562.

FIG. 35 depicts representative results demonstrating that degranulation of NKG2A+NK cells was reduced when co-cultured with HLA-E+ K562 cells. Degranulation of NKG2C+ NK cells increased when co-cultured with HLA-E+ K562 indicating NK cell activation.

FIG. 36 depicts representative results demonstrating editing strategy and efficiency of generating TKO HLA-E+ T cells.

FIG. 37 depicts representative results demonstrating TKO T cells were not recognized by allogeneic donor PBMCs as an alloreactive response was not measured in a mixed lymphocyte reaction (MLR).

FIG. 38 depicts representative results demonstrating TKO CAR T cells were targeted and killed by NK cells due to “missing-self” response. HLA-E expression on TKO CAR T cells inhibited NK cell mediated-lysis.

FIG. 39 depicts representative results demonstrating that HLA-E SCT expression demonstrated in vivo protection against NK activity.

### DETAILED DESCRIPTION

The present invention is based, in part, on the unexpected results that expression of modified human leukocyte antigen (HLA) class I histocompatibility antigen alpha chain E (HLA-E) and the deletion of beta-2-microglobulin (B2M), class II major histocompatibility complex transactivator (CIITA), and native T cell receptor (TCR) alpha chain inhibited natural killer (NK) cells and increased persistence of donor T cells in the host. Thus, in one aspect, the present invention provides a single chain trimer comprising a modified B2M, HLA-E, and HLA signal peptide or a fragment thereof that comprises at least one amino acid sequence that is at least about 70% identical to the amino acid sequence selected from SEQ ID NOs: 1-10, 13, and 46. In some embodiments, the single chain trimer comprises an amino acid sequence that is at least about 70% identical to the amino acid sequence set forth in SEQ ID NO: 40.

In another aspect, the present invention provides a nucleic acid molecule comprising a nucleotide sequence encoding a single chain trimer comprising a modified B2M, HLA-E, and HLA signal peptide or a fragment thereof that comprises at least one amino acid sequence that is at least about 70% identical to the amino acid sequence selected from SEQ ID NOs: 1-10, 13, and 46. In another aspect, the present invention provides a nucleic acid molecule comprising a nucleotide sequence encoding a modified B2M, nucleotide sequence encoding a HLA-E, and nucleotide sequence encoding a HLA signal peptide or a fragment thereof that comprises at least one nucleotide sequence encoding sequence that is at least about 70% identical to the nucleotide sequence selected from SEQ ID NOs: 14-24 and 47. In some embodiments, the nucleic acid molecule comprises a nucleotide sequence that is at least about 70% identical to the nucleotide sequence set forth in SEQ ID NO: 41. In various embodiments, the present invention also provides a composition and genetically engineered cell comprising at least nucleic acid molecule of the present invention.

In some aspects, the present invention provides a method of preventing, reducing, or eliminating an alloresponse, allorecognition of donor cells, or allograft or xenograft rejection, method of depleting the level of an alloresponsive cell, method of inducing an allogeneic tolerance, method of improving the effectiveness of a CAR cell therapy and/or engineered TCR cell therapy, and/or method of preventing or treating a disease or disorder in a subject in need thereof using at least one genetically engineered cell of the present invention. In some embodiments, the subject has an organ transplantation, tissue transplantation, cell transplantation, allotransplantation, intestinal transplantation, reconstructive transplantation, cancer, and/or disease or disorder associated with cancer.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or in some instances±10%, or in some instances±5%, or in some instances 10%, or in some instances±0.1% from the specified value, as such variations are appropriate to perform the disclosed methods.

The terms “patient,” “subject,” “individual,” and the like are used interchangeably herein, and refer to any animal, in some embodiments a mammal, and in some embodiments a human, having a complement system, including a human in need of therapy for, or susceptible to, a condition or its sequelae. The individual may include, for example, dogs, cats, pigs, cows, sheep, goats, horses, rats, monkeys, and mice and humans.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected/homeostatic) respective characteristic. Characteristics which are normal or expected for one cell, tissue type, or subject, might be abnormal for a different cell or tissue type.

A “disease” is a state of health of a subject wherein the subject cannot maintain homeostasis, and wherein if the disease is not ameliorated then the subject's health continues to deteriorate.

In contrast, a “disorder” in a subject is a state of health in which the subject is able to maintain homeostasis, but in which the subject's state of health is less favorable than it would be in the absence of the disorder. Left untreated, a disorder does not necessarily cause a further decrease in the subject's state of health.

“Cancer,” as used herein, refers to the abnormal growth or division of cells. Generally, the growth and/or life span of a cancer cell exceeds, and is not coordinated with, that of the normal cells and tissues around it. Cancers may be benign, pre-malignant or malignant. Cancer occurs in a variety of cells and tissues, including the oral cavity (e.g., mouth, tongue, pharynx, etc.), digestive system (e.g., esophagus, stomach, small intestine, colon, rectum, liver, bile duct, gall bladder, pancreas, etc.), respiratory system (e.g., larynx, lung, bronchus, etc.), bones, joints, skin (e.g., basal cell, squamous cell, meningioma, etc.), breast, genital system, (e.g., uterus, ovary, prostate, testis, etc.), urinary system (e.g., bladder, kidney, ureter, etc.), eye, nervous system (e.g., brain, etc.), endocrine system (e.g., thyroid, etc.), and hematopoietic system (e.g., lymphoma, myeloma, leukemia, acute lymphocytic leukemia, chronic lymphocytic leukemia, acute myeloid leukemia, chronic myeloid leukemia, etc.).

A disease or disorder is “alleviated” if the severity of a sign or symptom of the disease or disorder, the frequency with which such a sign or symptom is experienced by a patient, or both, is reduced.

“Activation”, as used herein, refers to the state of a T cell that has been sufficiently stimulated to induce detectable cellular proliferation. Activation can also be associated with induced cytokine production, and detectable effector functions. The term “activated T cells” refers to, among other things, T cells that are undergoing cell division.

The terms “inhibit” and “inhibition,” as used herein. means to reduce, suppress, diminish or block an activity or function by at least about 10% relative to a control value. In some embodiments, the activity is suppressed or blocked by at least about 50% compared to a control value. In some embodiments, the activity is suppressed or blocked by at least about 75%. In some embodiments, the activity is suppressed or blocked by at least about 95%.

As used herein, the term “autologous” is meant to refer to any material derived from the same individual to whom it is later to be re-introduced into the individual.

“Allogeneic” refers to any material derived from a different animal of the same species as the individual to whom the material is introduced. Two or more individuals are said to be allogeneic to one another when the genes at one or more loci are not identical. In some aspects, allogeneic material from individuals of the same species may be sufficiently unlike genetically to interact antigenically.

The phrase “disease associated with expression of expression of alloresponsive cells” as used herein includes, but is not limited to, a disease associated with expression of alloresponsive cells or condition associated with cells which express alloresponse including, e.g., an allograft rejection, an immune rejection, a chronic allogeneic rejection, an engraftment rejection, a transplant rejection, an inflammation, an inflammation caused by ischemia/reperfusion, an infection, an immune response to an allograft, and any combination thereof.

“Xenogeneic” refers to a graft derived from an animal of a different species.

As used herein “endogenous” refers to any material from or produced inside an organism, cell, tissue or system.

As used herein, the term “exogenous” refers to any material introduced from or produced outside an organism, cell, tissue or system.

As used herein, the term “immune cell” includes cells that are of hematopoietic origin and that play a role in the immune response. Immune cells include lymphocytes, such as B cells and T cells; natural killer cells; myeloid cells, such as monocytes, macrophages, dendritic cells, eosinophils, mast cells, basophils, and granulocytes.

As used herein, the term “immune response” includes T cell mediated and/or B cell mediated immune responses that are influenced by modulation of T cell co-stimulation. The term immune response further includes immune responses that are indirectly effected by T cell activation such as antibody production (humoral responses) and the activation of cytokine responsive cells such as macrophages.

As used herein, the term “T cell immune response” refers to activation of antigen specific T cells as measured by proliferation or expression of molecules on the cell surface or secretion of proteins such as cytokines.

As used herein, the term “T cell” refers to a lymphocyte (e.g., white blood cell) that functions in cell-mediated immunity. In some embodiments, the presence of a T cell receptor (TCR) on the cell surface distinguishes T cells from other lymphocytes. As is known in the art, T cells typically do not present antigens, and rely on other lymphocytes (e.g., natural killer cells and B cells) to aid in antigen presentation. Types of T cells include: T helper cells (TH cells), Memory T cells (Tcm, Tem, or Temra), Regulatory T cells (Treg), Cytotoxic T cells (CTLs), Natural killer T cells (NK cells), gamma delta T cells, and Mucosal associated invariant T cells (MAIT).

As used herein, the term “TCR” refers to “T cell receptor.” A T cell receptor is a molecule on the surface of T lymphocytes (“T cells”). In embodiments, the receptor is an αβ-TCR receptor, meaning that the T cell receptor comprises an alpha (α) and beta (β) chain, which is typically expressed as part of a complex with CD3 chain molecules.

As used herein, the term “B cell” refers to a cell produced in the bone marrow of an animal expressing membrane-bound antibody specific for an antigen. Following interaction with the antigen it differentiates into a plasma cell producing antibodies specific for the antigen or into a memory B cell. “B cell” and “B lymphocyte” is used interchangeably. Naïve as well as activated B cells are within the scope of the invention.

As used herein, the term “chimeric antigen receptor” or alternatively a “CAR” refers to a recombinant polypeptide construct comprising at least an extracellular antigen binding domain, a transmembrane domain, and a cytoplasmic signaling domain comprising a functional signaling domain derived from a stimulatory molecule as defined below. In one aspect, the stimulatory molecule is the zeta chain associated with the TCR complex. In one aspect, the cytoplasmic signaling domain further comprises one or more functional signaling domains derived from at least one costimulatory molecule as defined below. In one aspect, the costimulatory molecule is chosen from 4 1BB (i.e., CD137), CD3, and/or CD28. In one aspect, the CAR comprises a chimeric fusion protein comprising an extracellular antigen recognition domain, a transmembrane domain, and an intracellular signaling domain comprising a functional signaling domain derived from a stimulatory molecule. In one aspect, the CAR comprises a chimeric fusion protein comprising an extracellular antigen recognition domain, a transmembrane domain and an intracellular signaling domain comprising a functional signaling domain derived from a co-stimulatory molecule and a functional signaling domain derived from a stimulatory molecule. In one aspect, the CAR comprises a chimeric fusion protein comprising an extracellular antigen recognition domain, a transmembrane domain and an intracellular signaling domain comprising two functional signaling domains derived from one or more co-stimulatory molecule(s) and a functional signaling domain derived from a stimulatory molecule. In one aspect, the CAR comprises a chimeric fusion protein comprising an extracellular antigen recognition domain, a transmembrane domain and an intracellular signaling domain comprising at least two functional signaling domains derived from one or more co-stimulatory molecule(s) and a functional signaling domain derived from a stimulatory molecule. In one aspect the CAR comprises an optional leader sequence at the amino-terminus (N-ter) of the CAR fusion protein. In one aspect, the CAR further comprises a leader sequence at the N-terminus of the extracellular antigen recognition domain, wherein the leader sequence is optionally cleaved from the scFv domain during cellular processing and localization of the CAR to the cellular membrane.

The portion of the CAR composition comprising an antibody or antibody fragment thereof may exist in a variety of forms where the antigen binding domain is expressed as part of a contiguous polypeptide chain including, for example, a single domain antibody fragment (sdAb), a single chain antibody (scFv) and a humanized antibody (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, New York; Houston et al., 1988, Proc. Natl. Acad. Sci. USA 85:5879-5883; Bird et al., 1988, Science 242:423-426). In one aspect, the antigen binding domain of a CAR composition of the invention comprises an antibody fragment. In one embodiment, the CAR comprises an antibody fragment that comprises a scFv.

As used herein, a “signaling domain” is the functional portion of a protein which acts by transmitting information within the cell to regulate cellular activity via defined signaling pathways by generating second messengers or functioning as effectors by responding to such messengers.

The term “antibody,” as used herein, refers to a protein, or polypeptide sequence derived from an immunoglobulin molecule which specifically binds with an antigen. Antibodies can be polyclonal or monoclonal, multiple or single chain, or intact immunoglobulins, and may be derived from natural sources or from recombinant sources. Antibodies are typically tetramers of immunoglobulin molecules.

The term “antibody fragment” refers to at least one portion of an intact antibody, or recombinant variants thereof, and refers to the antigen binding domain, e.g., an antigenic determining variable region of an intact antibody, that is sufficient to confer recognition and specific binding of the antibody fragment to a target, such as an antigen. Examples of antibody fragments include, but are not limited to, Fab, Fab′, F(ab′)2, and Fv fragments, scFv antibody fragments, linear antibodies, single domain antibodies such as sdAb (either VL or VH), camelid VHH domains, and multi-specific antibodies formed from antibody fragments. The term “scFv” refers to a fusion protein comprising at least one antibody fragment comprising a variable region of a light chain and at least one antibody fragment comprising a variable region of a heavy chain, wherein the light and heavy chain variable regions are contiguously linked via a short flexible polypeptide linker, and capable of being expressed as a single chain polypeptide, and wherein the scFv retains the specificity of the intact antibody from which it is derived. Unless specified, as used herein an scFv may have the VL and VH variable regions in either order, e.g., with respect to the N-terminal and C-terminal ends of the polypeptide, the scFv may comprise VL-linker-VH or may comprise VH-linker-VL.

An “antibody heavy chain,” as used herein, refers to the larger of the two types of polypeptide chains present in antibody molecules in their naturally occurring conformations, and which normally determines the class to which the antibody belongs.

An “antibody light chain,” as used herein, refers to the smaller of the two types of polypeptide chains present in antibody molecules in their naturally occurring conformations. Kappa and lambda light chains refer to the two major antibody light chain isotypes.

By the term “recombinant antibody” as used herein, is meant an antibody which is generated using recombinant DNA technology, such as, for example, an antibody expressed by a bacteriophage or yeast expression system. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using recombinant DNA or amino acid sequence technology which is available and well known in the art.

The term “antigen” or “Ag” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to encode polypeptides that elicit the desired immune response. Moreover, a skilled artisan will understand that an antigen need not be encoded by a “gene” at all. It is readily apparent that an antigen can be generated synthesized or can be derived from a biological sample, or might be macromolecule besides a polypeptide. Such a biological sample can include, but is not limited to a tissue sample, a cell or a fluid with other biological components.

An “antigen presenting cell” or “APC” as used herein, means an immune system cell such as an accessory cell (e.g., a B-cell, a dendritic cell, and the like) that displays foreign antigens complexed with HLA I, HLA II, MHC I, MHC II, or MHC III complexes on their surfaces. For example, T cells may recognize these complexes using their T-cell receptors (TCRs). APCs process antigens and present them to T-cells.

By the term “stimulation,” is meant a primary response induced by binding of a stimulatory molecule (e.g., a TCR complex, BCR complex, etc.) with its cognate ligand thereby mediating a signal transduction event, such as, but not limited to, signal transduction via the TCR complex and/or BCR complex, etc. Stimulation can mediate altered expression of certain molecules, such as downregulation of TGF-β, and/or reorganization of cytoskeletal structures, and the like.

A “stimulatory molecule,” as the term is used herein, means a molecule expressed by a cell that provide the primary cytoplasmic signaling sequence(s) that regulate primary activation of the TCR and/or BCR complex in a stimulatory way for at least some aspect of the cell signaling pathway. In one aspect, the primary signal is initiated by, for instance, binding of a TCR and/or BCR complex with a human leukocyte antigen (HLA) I, HLA II, major histocompatibility complex (MHC) I, MHC II, or MHC III molecule loaded with peptide, and which leads to mediation of a cell response, including, but not limited to, proliferation, activation, differentiation, and the like. Primary cytoplasmic signaling sequences that act in a stimulatory manner may contain signaling motifs which are known as immunoreceptor tyrosine-based activation motifs or ITAMs. Examples of ITAM containing primary cytoplasmic signaling sequences that are of use in the invention include those derived from TCR zeta, FcR gamma, FcR beta, CD3 gamma, CD3 delta, CD3 epsilon, CD5, CD22, CD79a, CD79b, CD278 (also known as “ICOS”) and CD66d. In a specific CAR of the invention, the cytoplasmic signaling sequence derived from CD3-zeta is derived from a non-human species, e.g., mouse, rodent, monkey, ape and the like.

The terms “effective amount” and “pharmaceutically effective amount” refer to a sufficient amount of an agent to provide the desired biological result. That result can be reduction and/or alleviation of the signs, symptoms, or causes of a disease or disorder, or any other desired alteration of a biological system. An appropriate effective amount in any individual case may be determined by one of ordinary skill in the art using routine experimentation.

The term “therapeutic” as used herein means a treatment. A therapeutic effect is obtained by reduction, suppression, remission, or eradication of a disease state.

A “therapeutic treatment” is a treatment administered to a subject who exhibits signs of disease or disorder, for the purpose of diminishing or eliminating those signs.

As used herein, “treating a disease or disorder” means reducing the frequency and/or severity of a sign and/or symptom of the disease or disorder is experienced by a patient.

The term “prophylaxis” as used herein means the prevention of or protective treatment for a disease or disease state.

The phrase “biological sample”, “sample” or “specimen” as used herein, is intended to include any sample comprising a cell, a tissue, or a bodily fluid in which expression of a nucleic acid or polypeptide can be detected. The biological sample may contain any biological material suitable for detecting the desired biomarkers, and may comprise cellular and/or non-cellular material obtained from the individual. Examples of such biological samples include but are not limited to blood, lymph, bone marrow, biopsies and smears. Samples that are liquid in nature are referred to herein as “bodily fluids.” Biological samples may be obtained from a patient by a variety of techniques including, for example, by scraping or swabbing an area or by using a needle to obtain bodily fluids. Methods for collecting various body samples are well known in the art.

“CDRs” are defined as the complementarity determining region amino acid sequences of a TCR or TCR chain.

As used herein, an “immunoassay” refers to any binding assay that uses an antibody capable of binding specifically to a target molecule to detect and quantify the target molecule.

By the term “specifically binds,” as used herein with respect to a polypeptide (e.g., a TCR or TCR chain), is meant a polypeptide which recognizes and binds to a specific target molecule, but does not substantially recognize or bind other molecules in a sample. In some instances, the terms “specific binding” or “specifically binding,” is used to mean that the recognition and binding is dependent upon the presence of a particular structure (e.g., an antigenic determinant or epitope) on the target molecule.

A “coding region” of a gene consists of the nucleotide residues of the coding strand of the gene and the nucleotides of the non-coding strand of the gene which are homologous with or complementary to, respectively, the coding region of an mRNA molecule which is produced by transcription of the gene. A “coding region” of a mRNA molecule also consists of the nucleotide residues of the mRNA molecule which are matched with an anti-codon region of a transfer RNA molecule during translation of the mRNA molecule or which encode a stop codon. The coding region may thus include nucleotide residues comprising codons for amino acid residues which are not present in the mature protein encoded by the mRNA molecule (e.g., amino acid residues in a protein export signal sequence).

“Complementary” as used herein to refer to a nucleic acid, refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. In some embodiments, the first region comprises a first portion and the second region comprises a second portion, whereby, when the first and second portions are arranged in an antiparallel fashion, at least about 50%, and or at least about 75%, or at least about 90%, or at least about 95% of the nucleotide residues of the first portion are capable of base pairing with nucleotide residues in the second portion. In some embodiments, all nucleotide residues of the first portion are capable of base pairing with nucleotide residues in the second portion.

“Homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared×100. For example, if 6 of 10 of the positions in two sequences are matched or homologous then the two sequences are 60% homologous. Generally, a comparison is made when two sequences are aligned to give maximum homology.

“Variant” as the term is used herein, is a nucleic acid sequence or a peptide sequence that differs in sequence from a reference nucleic acid sequence or peptide sequence respectively, but retains essential biological properties of the reference molecule. Changes in the sequence of a nucleic acid variant may not alter the amino acid sequence of a peptide encoded by the reference nucleic acid, or may result in amino acid substitutions, additions, deletions, fusions and truncations. Changes in the sequence of peptide variants are typically limited or conservative, so that the sequences of the reference peptide and the variant are closely similar overall and, in many regions, identical. A variant and reference peptide can differ in amino acid sequence by one or more substitutions, additions, deletions in any combination. A variant of a nucleic acid or peptide can be a naturally occurring such as an allelic variant, or can be a variant that is not known to occur naturally. Non-naturally occurring variants of nucleic acids and peptides may be made by mutagenesis techniques or by direct synthesis. In various embodiments, the variant sequence is at least 99%, at least 98%, at least 97%, at least 96%, at least 95%, at least 94%, at least 93%, at least 92%, at least 91%, at least 90%, at least 89%, at least 88%, at least 87%, at least 86%, at least 85% identical to the reference sequence.

The term “DNA” as used herein is defined as deoxyribonucleic acid.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting there from. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

“Isolated” means altered or removed from the natural state. For example, a nucleic acid or a peptide naturally present in its normal context in a living subject is not “isolated,” but the same nucleic acid or peptide partially or completely separated from the coexisting materials of its natural context is “isolated.” An isolated nucleic acid or protein can exist in substantially purified form, or can exist in a non-native environment such as, for example, a host cell.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “RNA” as used herein is defined as ribonucleic acid.

The term “recombinant DNA” as used herein is defined as DNA produced by joining pieces of DNA from different sources.

The term “recombinant polypeptide” as used herein is defined as a polypeptide produced by using recombinant DNA methods.

As used herein, “conjugated” refers to covalent attachment of one molecule to a second molecule.

“Operably linked” or “operatively linked” as used herein may mean that expression of a gene is under the control of a promoter with which it is spatially connected. A promoter may be positioned 5′ (upstream) or 3′ (downstream) of a gene under its control. The distance between the promoter and a gene may be approximately the same as the distance between that promoter and the gene it controls in the gene from which the promoter is derived. As is known in the art, variation in this distance may be accommodated without loss of promoter function.

The term “promoter” as used herein is defined as a DNA sequence recognized by the synthetic machinery of the cell, or introduced synthetic machinery, required to initiate the specific transcription of a polynucleotide sequence.

The term “regulating” as used herein can mean any method of altering the level or activity of a substrate. Non-limiting examples of regulating with regard to a protein include affecting expression (including transcription and/or translation), affecting folding, affecting degradation or protein turnover, and affecting localization of a protein. Non-limiting examples of regulating with regard to an enzyme further include affecting the enzymatic activity. “Regulator” refers to a molecule whose activity includes affecting the level or activity of a substrate. A regulator can be direct or indirect. A regulator can function to activate or inhibit or otherwise modulate its substrate.

“Vector” as used herein may mean a nucleic acid sequence containing an origin of replication. A vector may be a plasmid, bacteriophage, bacterial artificial chromosome or yeast artificial chromosome. A vector may be a DNA or RNA vector. A vector may be either a self-replicating extrachromosomal vector or a vector which integrates into a host genome.

As used herein, a “substantially purified” cell is a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some embodiments, the cells are cultured in vitro. In other embodiments, the cells are not cultured in vitro.

“Knockout” means having a specific single gene or allele(s) of a gene disrupted from a genome by genetic manipulation. Accordingly, a “single-allele, knockout cell” refers to a cell in which a single allele of a gene has been disrupted such that its gene product is not expressed.

The term “knockout construct” refers to a nucleic acid sequence that is designed to decrease or suppress expression of a protein encoded by endogenous DNA sequences in a cell. The nucleic acid sequence used as the knockout construct is typically comprised of (1) DNA from some portion of the gene (exon sequence, intron sequence, and/or promoter sequence) to be suppressed and (2) a marker sequence used to detect the presence of the knockout construct in the cell. The knockout construct is inserted into a cell, and integrates with the genomic DNA of the cell in such a position so as to prevent or interrupt transcription of the native DNA sequence. Such insertion usually occurs by homologous recombination (i.e., regions of the knockout construct that are homologous to endogenous DNA sequences hybridize to each other when the knockout construct is inserted into the cell and recombine so that the knockout construct is incorporated into the corresponding position of the endogenous DNA). The knockout construct nucleic acid sequence may comprise 1) a full or partial sequence of one or more exons and/or introns of the gene to be suppressed, 2) a full or partial promoter sequence of the gene to be suppressed, or 3) combinations thereof.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention provides a single chain trimer comprising a modified B2M, HLA-E, and HLA signal peptide or a fragment thereof that comprises at least one amino acid sequence that is at least about 70% identical to the amino acid sequence selected from SEQ ID NOs: 1-10, 13, and 46. In some embodiments, the single chain trimer comprises an amino acid sequence that is at least about 70% identical to the amino acid sequence set forth in SEQ ID NO: 40. In another aspect, the present invention provides a nucleic acid molecule comprising a nucleotide sequence encoding a single chain trimer comprising a modified B2M, HLA-E, and HLA signal peptide or a fragment thereof that comprises at least one amino acid sequence that is at least about 70% identical to the amino acid sequence selected from SEQ ID NOs: 1-10, 13, and 46. In another aspect, the present invention provides a nucleic acid molecule comprising a nucleotide sequence encoding a modified B2M, nucleotide sequence encoding a HLA-E, and nucleotide sequence encoding a HLA signal peptide or a fragment thereof that comprises at least one nucleotide sequence encoding sequence that is at least about 70% identical to the nucleotide sequence selected from SEQ ID NOs: 14-24 and 47. In some embodiments, the nucleic acid molecule comprises a nucleotide sequence that is at least about 70% identical to the nucleotide sequence set forth in SEQ ID NO: 41. In various embodiments, the present invention also provides a composition and genetically engineered cell comprising at least nucleic acid molecule of the present invention.

In some aspects, the present invention provides a method of preventing, reducing, or eliminating an alloresponse, allorecognition of donor cells, or allograft or xenograft rejection, method of depleting the level of an alloresponsive cell, method of inducing an allogeneic tolerance, method of improving the effectiveness of a CAR therapy, TCR therapy, CAR cell therapy, and/or engineered TCR cell therapy method of preventing or treating a disease or disorder in a subject in need thereof using at least one genetically engineered cell of the present invention. In some embodiments, the subject has an organ transplantation, tissue transplantation, cell transplantation, allotransplantation, intestinal transplantation, reconstructive transplantation, cancer, and/or disease or disorder associated with cancer.

### Peptides, Nucleic Acid Molecules, and Compositions

In one aspect, the present invention relates, in part to a peptide for reducing clearance or increasing persistence of at least one cell of interest, the peptide comprising an HLA signal peptide or a fragment thereof, a modified B2M or a fragment thereof, and an HLA-E or a fragment thereof. In some embodiments, the peptide is a single chain trimer. In another aspect, the present invention provides a nucleic acid molecule for reducing clearance or increasing persistence of at least one cell of interest, the nucleic acid molecule comprising a nucleotide sequence that encodes a peptide comprising an HLA signal peptide or a fragment thereof, a modified B2M or a fragment thereof, and an HLA-E or a fragment thereof. In another aspect, a nucleic acid molecule for reducing clearance or increasing persistence of at least one cell of interest, the nucleic acid molecule comprising a nucleotide sequence that encodes an HLA signal peptide or a fragment thereof, a nucleotide sequence that encodes a modified B2M or a fragment thereof, and a nucleotide sequence that encodes an HLA-E or a fragment thereof. In various embodiments, the at least one cell of interest is a donor cell, such as a T cell, CAR T cell, engineered TCR-expressing T cell, etc.

In some embodiments, the HLA-E inhibits an NK cell-mediated killing of a donor cell. In some embodiments, the HLA-E comprises a modified HLA-E or a fragment thereof. In some embodiments, the HLA-E comprises a human HLA-E or a fragment thereof. In some embodiments, the HLA-E comprises a HLA-E*0101 or a fragment thereof and/or HLA-E*0103 or a fragment thereof. For example, in some embodiments, the HLA-E comprises the 64 to 1077 amino acid segment of HLA-E*0101 set forth in SEQ ID NO: 37. In some embodiments, the HLA-E comprises the amino acid sequence of HLA-E*0103 set forth in SEQ ID NO: 43.

In some embodiments, the modified B2M comprises a B2M signal peptide or a fragment thereof and/or leader-less B2M or a fragment thereof. In one embodiment, the B2M signal peptide is a modified B2M signal peptide set forth in SEQ ID NO: 25. In some embodiment, the leader-less B2M is a modified leader-less B2M set forth in SEQ ID NO: 31.

In some embodiments, the modified B2M or fragment thereof is linked to the HLA-E or fragment thereof, the HLA signal peptide or fragment thereof, or any combination thereof. In some embodiments, the modified B2M or fragment thereof is directly linked to the HLA-E or fragment thereof, the HLA signal peptide or fragment thereof, or any combination thereof. In some embodiments, the modified B2M or fragment thereof is linked to the HLA-E or fragment thereof, the HLA signal peptide or fragment thereof, or any combination thereof via a linker and/or a spacer.

In some embodiments, the nucleotide sequence encoding the modified B2M or fragment thereof is linked to the nucleotide sequence encoding the HLA-E or fragment thereof, the nucleotide sequence encoding the HLA signal peptide or fragment thereof, or any combination thereof. In some embodiments, the nucleotide sequence encoding the modified B2M or fragment thereof is directly linked to the nucleotide sequence encoding the HLA-E or fragment thereof, the nucleotide sequence encoding the HLA signal peptide or fragment thereof, or any combination thereof. In some embodiments, the nucleotide sequence encoding the modified B2M or fragment thereof is linked to the nucleotide sequence encoding the HLA-E or fragment thereof, the nucleotide sequence encoding the HLA signal peptide or fragment thereof, or any combination thereof via a linker and/or a spacer.

In various embodiments, the linker is any linker known in the art. In one embodiment, the linker is not a cleavable linker. In one embodiment, the linker is a cleavable linker. In some embodiment, the cleavable linker is a chemically cleavable linker, enzyme cleavable linker, peptide-based linker, or any combination thereof. In some embodiments, the chemically cleavable linker is an acid-cleavable linkers or reducible linker. In one embodiment, the acid-cleavable linker is specifically designed to remain stable at the neutral pH of blood circulation, but undergo hydrolysis and release the cytotoxic drug in the acidic environment of the cellular compartments. In one embodiment, the reducible linker is designed to remain stable in the oxygen-rich environment in the bloodstream, and is selectively cleaved in the reducing environment of the cell. In another embodiment, the peptide-based linker is designed to keep intact in systemic circulation, and to be cleaved by specific intracellular proteases, such as cathepsin B. Examples of linkers include, but are not limited to, linkers containing a lysosomal-specific protease cleavage site, linkers containing mixed disulfides, aminoethoxyethoxyacetate (AEEA), β-glucuronide linker, peptide linkers cleavable by intracellular proteases, e.g., lysosomal proteases or endosomal proteases, dipeptide linker (e.g., valine-citrulline (val-cit) or a phenylalanine-lysine (phe-lys) linker), aninohexanoate, hydrazone, thiomaleimide, and dibenzocyclooctyne (DBCO). For example, in some embodiments, the linker comprises an amino acid set forth in SEQ ID NO: 28. In some embodiments, the linker comprises a nucleotide sequence set forth in SEQ ID Nos: 29-30. In some embodiments, the linker comprises a nucleotide sequence set forth in SEQ ID No: 29.

In various embodiments, the spacer is any spacer known in the art. For example, in some embodiments, the spacer comprises an amino acid set forth in SEQ ID NO: 34. In some embodiments, the linker comprises a nucleotide sequence set forth in SEQ ID Nos: 35-36. In some embodiments, the linker comprises a nucleotide sequence set forth in SEQ ID No: 35.

In some embodiments, the peptide comprises a modB2M signal peptide (for example SEQ ID NO: 25), an HLA signal peptide described herein (for example SEQ ID NOs: 1-10, 13, or 46), a leader-less modB2M (for example SEQ ID NO: 31), and HLA-E (for example SEQ ID NOs: 37 or 44). In one embodiment, the peptide comprises a linker or spacer between the HLA signal peptide and the leader-less modB2M. In one embodiment the peptide comprises a linker or spacer between the leader-less modB2M and HLA-E.

For example, in some embodiments, the modified B2M signal peptide set forth in SEQ ID NO: 25 or fragment thereof is directly linked to the HLA signal peptide set forth in SEQ ID NO: 3 or fragment thereof; the HLA signal peptide or fragment is linked to the leader-less modified B2M set forth in SEQ ID NO: 31 or fragment thereof via a linker set forth in SEQ ID NO: 28; and the leader-less modified B2M or fragment thereof is linked to the HLA-E set forth in SEQ ID NO: 37 or fragment thereof via a spacer set forth in SEQ ID NO: 34.

In other embodiments, the modified B2M signal peptide set forth in SEQ ID NO: 25 or fragment thereof is directly linked to the HLA signal peptide set forth in SEQ ID NO: 3 or fragment thereof; the HLA signal peptide or fragment is linked to the leader-less modified B2M set forth in SEQ ID NO: 31 or fragment thereof via a linker set forth in SEQ ID NO: 28; and the leader-less modified B2M or fragment thereof is linked to the HLA-E set forth in SEQ ID NO: 44 or fragment thereof via a spacer set forth in SEQ ID NO: 34.

In some embodiments, the nucleic acid molecule encoding the peptide of the present invention comprises a nucleotide sequence encoding the modB2M signal peptide (for example SEQ ID NOs: 26 or 27), a nucleotide sequence encoding the HLA signal peptide described herein (for example SEQ ID NOs: 14-24, 43, or 47), a nucleotide sequence encoding the leader-less modB2M (for example SEQ ID NOs: 32 or 33), and a nucleotide sequence encoding the HLA-E (for example SEQ ID NOs: 38, 39, or 45). In one embodiment, the nucleic acid molecule comprises a linker or spacer between the nucleotide sequence encoding the HLA signal peptide and the nucleotide sequence encoding the leader-less modB2M. In one embodiment the nucleic acid molecule comprises a linker or spacer between the nucleotide sequence encoding the leader-less modB2M and the nucleotide sequence encoding the HLA-E.

For example, in some embodiments, the nucleotide sequence encoding the modified B2M signal peptide set forth in SEQ ID NO: 26 or fragment thereof is directly linked to the nucleotide sequence encoding the HLA signal peptide set forth in SEQ ID NO: 14 or fragment thereof; the nucleotide sequence encoding the HLA signal peptide or fragment is linked to the nucleotide sequence encoding the leader-less modified B2M set forth in SEQ ID NO: 32 or fragment thereof via a linker set forth in SEQ ID NO: 29; and the nucleotide sequence encoding the leader-less modified B2M or fragment thereof is linked to the nucleotide sequence encoding the HLA-E set forth in SEQ ID NO: 38 or fragment thereof via a spacer set forth in SEQ ID NO: 35.

In some embodiments, the nucleotide sequence encoding the modified B2M signal peptide set forth in SEQ ID NO: 26 or fragment thereof is directly linked to the nucleotide sequence encoding the HLA signal peptide set forth in SEQ ID NO: 14 or fragment thereof; the nucleotide sequence encoding the HLA signal peptide or fragment is linked to the nucleotide sequence encoding the leader-less modified B2M set forth in SEQ ID NO: 32 or fragment thereof via a linker set forth in SEQ ID NO: 29; and the nucleotide sequence encoding the leader-less modified B2M or fragment thereof is linked to the nucleotide sequence encoding the HLA-E set forth in SEQ ID NO: 45 or fragment thereof via a spacer set forth in SEQ ID NO: 35.

In one embodiment, the HLA signal peptide or the fragment thereof has a length of about 8 to about 24 amino acid residues, or about 9 to about 11 amino acid residues. In an embodiment of the invention, the modified HLA or the fragment thereof has a length of about 8 amino acid residues, about 9 amino acid residues, about 10 amino acid residues, about 11 amino acid residues, about 12 amino acid residues, about 13 amino acid residues, about 14 amino acid residues, about 15 amino acid residues, about 16 amino acid residues, about 17 amino acid residues, about 18 amino acid residues, about 19 amino acid residues, about 20 amino acid residues, about 21 amino acid residues, about 22 amino acid residues, about 23 amino acid residues, or about 24 amino acid residues.

In some embodiments, the HLA signal peptide or a fragment thereof is an HLA-A signal peptide or a fragment thereof, HLA-A2 signal peptide or a fragment thereof, HLA-B signal peptide or a fragment thereof, HLA-B5 signal peptide or a fragment thereof, HLA-C signal peptide or a fragment thereof, HLA-DP signal peptide or a fragment thereof, HLA-DPA1 signal peptide or a fragment thereof, HLA-DPB1 signal peptide or a fragment thereof, HLA-DQ signal peptide or a fragment thereof, HLA-DQA1 signal peptide or a fragment thereof, HLA-DQA2 signal peptide or a fragment thereof, HLA-DQB1 signal peptide or a fragment thereof, HLA-DQB2 signal peptide or a fragment thereof, HLA-DR signal peptide or a fragment thereof, HLA-DR3 signal peptide or a fragment thereof, HLA-DRA1 signal peptide or a fragment thereof, HLA-DRB1 signal peptide or a fragment thereof, HLA-DRB2 signal peptide or a fragment thereof, HLA-DRB3 signal peptide or a fragment thereof, HLA-DRB4 signal peptide or a fragment thereof, HLA-DRB5 signal peptide or a fragment thereof, HLA-DRB6 signal peptide or a fragment thereof, HLA-DRB7 signal peptide or a fragment thereof, HLA-DRB8 signal peptide or a fragment thereof, HLA-DRB9 signal peptide or a fragment thereof, HLA-E signal peptide or a fragment thereof, HLA-F signal peptide or a fragment thereof, HLA-G signal peptide or a fragment thereof, or any combination thereof.

In some embodiments, the HLA signal peptide or a fragment thereof is a modified HLA signal peptide or a fragment thereof. For example, in some embodiments, the HLA signal peptide comprises at least one selected from an HLA-A*02:01 signal peptide or a fragment thereof, HLA-B*08:01 signal peptide or a fragment thereof, HLA-C*03:01 signal peptide or a fragment thereof, HLA-G signal peptide or a fragment thereof, HSP60 signal peptide or a fragment thereof, CMV Towne signal peptide or a fragment thereof, CMV AFT signal peptide or a fragment thereof, CMV 109b signal peptide or a fragment thereof, RL9HIV signal peptide or a fragment thereof, and Mtb44 signal peptide or a fragment thereof.

In some embodiments, the HLA signal peptide comprises at least one amino acid sequence selected from SEQ ID NOs: 1-10, 13, and 46, or fragments thereof. In some embodiments, the HLA signal peptide comprises at least one amino acid sequence that is substantially homologous to the amino acid sequence selected from SEQ ID NOs: 1-10, 13, and 46, or fragments thereof. For example, in certain embodiments, the amino acid sequence has a degree of identity with respect to the original amino acid sequence of at least about 60%, of at least about 65%, of at least about 70%, of at least about 75%, of at least about 80%, of at least about 85%, of at least about 90%, of at least about 91%, of at least about 92%, of at least about 93%, of at least about 94%, of at least about 95%, of at least about 96%, of at least about 97%, of at least about 98%, of at least about 99%, or of at least about 99.5% to the amino acid sequence selected from SEQ ID NOs: 1-10, 13, and 46, or fragments thereof.

In certain embodiments, the HLA signal peptide comprises an amino acid sequence that has one or more, two or more, three or more, four or more, five or more, six or more, seven or more, eight or more, nine or more, or ten or more mutations, such as point mutations, relative to an amino acid sequence selected from SEQ ID NOs: 1-10, 13, and 46.

In some embodiments, the peptide of the invention (i.e., the peptide comprising an HLA signal peptide or a fragment thereof, a modified B2M or a fragment thereof, and an HLA-E or a fragment thereof) comprises at least one amino acid sequence as set forth in SEQ ID NOs: 1-10, 13, 25, 28, 31, 34, 37, 40, 44, and 46 or a fragment thereof. In some embodiments, the peptide of the invention comprises at least one amino acid sequence that is substantially homologous to the amino acid sequence selected from SEQ ID NOs: 1-10, 13, 25, 28, 31, 34, 37, 40, 44, and/or 46, or a fragment thereof. For example, in certain embodiments, the amino acid sequence has a degree of identity with respect to the original amino acid sequence of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5% to the amino acid sequence selected from SEQ ID NOs: 1-10, 13, 25, 28, 31, 34, 37, 40, 44, and/or 46 or a fragment thereof.

As known in the art the “similarity” between two polypeptides is determined by comparing the amino acid sequence and its conserved amino acid substitutes of one polypeptide to a sequence of a second polypeptide. Variants are defined to include polypeptide sequences different from the original sequence, for example different from the original sequence in less than 40% of residues per segment of interest, or different from the original sequence in less than 25% of residues per segment of interest, or different by less than 10% of residues per segment of interest, or different from the original protein sequence in just a few residues per segment of interest and at the same time sufficiently homologous to the original sequence to preserve the functionality of the original sequence and/or the ability to bind to ubiquitin or to a ubiquitylated protein. The present invention includes amino acid sequences that are at least 60%, 65%, 70%, 72%, 74%, 76%, 78%, 80%, 90%, or 95% similar or identical to the original amino acid sequence. The degree of identity between two polypeptides is determined using computer algorithms and methods that are widely known for the persons skilled in the art. The identity between two amino acid sequences can be determined, by way of one example, by using the BLASTP algorithm (BLAST Manual, Altschul, S., et al., NCBI NLM NIH Bethesda, Md. 20894, Altschul, S., et al., J. Mol. Biol. 215: 403-410 (1990)).

The peptides of the invention can be post-translationally modified. For example, post-translational modifications that fall within the scope of the present invention include signal peptide cleavage, glycosylation, acetylation, isoprenylation, proteolysis, myristoylation, protein folding and proteolytic processing, etc. Some modifications or processing events require introduction of additional biological machinery. For example, processing events, such as signal peptide cleavage and core glycosylation, are examined by adding canine microsomal membranes or Xenopus egg extracts (U.S. Pat. No. 6,103,489) to a standard translation reaction.

The peptides of the invention may include unnatural amino acids formed by post-translational modification or by introducing unnatural amino acids during translation. A variety of approaches are available for introducing unnatural amino acids during protein translation. By way of example, special tRNAs, such as tRNAs which have suppressor properties, suppressor tRNAs, have been used in the process of site-directed non-native amino acid replacement (SNAAR). In SNAAR, a unique codon is required on the mRNA and the suppressor tRNA, acting to target a non-native amino acid to a unique site during the protein synthesis (described in WO 90/05785). However, the suppressor tRNA must not be recognizable by the aminoacyl tRNA synthetases present in the protein translation system. In certain cases, a non-native amino acid can be formed after the tRNA molecule is aminoacylated using chemical reactions which specifically modify the native amino acid and do not significantly alter the functional activity of the aminoacylated tRNA. These reactions are referred to as post-aminoacylation modifications. For example, the epsilon-amino group of the lysine linked to its cognate tRNA (tRNALYS), could be modified with an amine specific photoaffinity label.

The peptide of the present invention may be made using chemical methods. For example, peptides can be synthesized by solid phase techniques (Roberge J Y et al (1995) Science 269: 202-204), cleaved from the resin, and purified by preparative high performance liquid chromatography. Automated synthesis may be achieved, for example, using the ABI 431 A Peptide Synthesizer (Perkin Elmer) in accordance with the instructions provided by the manufacturer. The peptide may alternatively be made by recombinant means or by cleavage from a longer polypeptide. The composition of a peptide may be confirmed by amino acid analysis or sequencing.

The variants of the peptides according to the present invention may be (i) one in which one or more of the amino acid residues are substituted with a conserved or non-conserved amino acid residue and such substituted amino acid residue may or may not be one encoded by the genetic code, (ii) one in which there are one or more modified amino acid residues, e.g., residues that are modified by the attachment of substituent groups, (iii) one in which the polypeptide is an alternative splice variant of the polypeptide of the present invention, (iv) fragments of the polypeptides and/or (v) one in which the polypeptide is fused with another polypeptide, such as a leader or secretory sequence or a sequence which is employed for purification (for example, His-tag) or for detection (for example, Sv5 epitope tag). The fragments include polypeptides generated via proteolytic cleavage (including multi-site proteolysis) of an original sequence. Variants may be post-translationally, or chemically modified. Such variants are deemed to be within the scope of those skilled in the art from the teaching herein.

The peptides of the invention may be converted into pharmaceutical salts by reacting with inorganic acids such as hydrochloric acid, sulfuric acid, hydrobromic acid, phosphoric acid, etc., or organic acids such as formic acid, acetic acid, propionic acid, glycolic acid, lactic acid, pyruvic acid, oxalic acid, succinic acid, malic acid, tartaric acid, citric acid, benzoic acid, salicylic acid, benezenesulfonic acid, and toluenesulfonic acids.

In some embodiments, the nucleotide sequence encoding the HLA signal peptide comprises at least one nucleotide sequence that encodes the amino acid sequence selected from SEQ ID NOs: 1-10, 13, and 46, or fragments thereof. In some embodiments, the nucleotide sequence encoding the HLA signal peptide comprises at least one nucleotide sequence encoding an amino acid sequence that is substantially homologous to the amino acid sequence selected from SEQ ID NOs: 1-10, 13, and 46, or fragments thereof. For example, in certain embodiments, the nucleotide sequence encoding the HLA signal peptide comprises at least one nucleotide sequence encoding the amino acid sequence having a degree of identity with respect to the original amino acid sequence of at least about 60%, of at least about 65%, of at least about 70%, of at least about 75%, of at least about 80%, of at least about 85%, of at least about 90%, of at least about 91%, of at least about 92%, of at least about 93%, of at least about 94%, of at least about 95%, of at least about 96%, of at least about 97%, of at least about 98%, of at least about 99%, or of at least about 99.5% to the amino acid sequence selected from SEQ ID NOs: 1-10, 13, and 46, or fragments thereof.

In certain embodiments, the nucleotide sequence encoding the HLA signal peptide comprises at least one nucleotide sequence that encodes an amino acid sequence that has one or more, two or more, three or more, four or more, five or more, six or more, seven or more, eight or more, nine or more, or ten or more mutations, such as point mutations, relative to an amino acid sequence selected from SEQ ID NOs: 1-10, 13, and 46.

In some embodiments, the nucleic acid molecule of the invention (i.e., the nucleic acid molecule comprising a nucleotide sequence that encodes a peptide comprising an HLA signal peptide or a fragment thereof, a modified B2M or a fragment thereof, and an HLA-E or a fragment thereof or the nucleic acid molecule comprising a nucleotide sequence that encodes an HLA signal peptide or a fragment thereof, a nucleotide sequence that encodes a modified B2M or a fragment thereof, and a nucleotide sequence that encodes an HLA-E or a fragment thereof) comprises at least one nucleotide sequence encoding at least one amino acid sequence as set forth in SEQ ID NOs: 1-10, 13, 25, 28, 31, 34, 37, and 40 or a fragment thereof. In some embodiments, the nucleic acid molecule of the invention comprises at least one nucleotide sequence encoding amino acid sequence that is substantially homologous to the amino acid sequence selected from SEQ ID NOs: 1-10, 13, 25, 28, 31, 34, 37, 40, 44, and/or 46, or a fragment thereof. For example, in certain embodiments, the nucleotide sequence encodes the amino acid sequence has a degree of identity with respect to the original amino acid sequence of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5% to the amino acid sequence selected from SEQ ID NOs: 1-10, 13, 25, 28, 31, 34, 37, 40, 44, and/or 46, or a fragment thereof.

In some embodiments, the nucleotide sequence encoding the HLA signal peptide comprises at least one nucleotide sequence selected from SEQ ID NOs: 14-24, 43, and 47, or fragments thereof. In some embodiments, the nucleotide sequence encoding the HLA signal peptide comprises at least one nucleotide sequence comprises at least one nucleotide sequence that is substantially homologous to the nucleotide sequence selected from SEQ ID NOs: 14-24, 43, and 47. For example, in certain embodiments, the nucleotide sequence has a degree of identity with respect to the original nucleotide sequence of at least about 60%, of at least about 65%, of at least about 70%, of at least about 75%, of at least about 80%, of at least about 85%, of at least about 90%, of at least about 91%, of at least about 92%, of at least about 93%, of at least about 94%, of at least about 95%, of at least about 96%, of at least about 97%, of at least about 98%, of at least about 99%, or of at least about 99.5% to the nucleotide sequence selected from SEQ ID NOs: 14-24, 43, and 47, or fragments thereof.

In certain embodiments, the nucleotide sequence encoding the HLA signal peptide comprises a nucleotide sequence that has one or more, two or more, three or more, four or more, five or more, six or more, seven or more, eight or more, nine or more, or ten or more mutations, such as point mutations, relative to a nucleotide sequence selected from SEQ ID NOs: 14-24, 43, and 47.

In some embodiments, the nucleic acid molecule of the invention (i.e., the nucleic acid molecule comprising a nucleotide sequence that encodes a peptide comprising an HLA signal peptide or a fragment thereof, a modified B2M or a fragment thereof, and an HLA-E or a fragment thereof or the nucleic acid molecule comprising a nucleotide sequence that encodes an HLA signal peptide or a fragment thereof, a nucleotide sequence that encodes a modified B2M or a fragment thereof, and a nucleotide sequence that encodes an HLA-E or a fragment thereof) comprises at least one nucleotide sequence selected from SEQ ID NOs: 14-24, 26, 27, 29, 30, 32, 33, 35, 36, 38, 39, 41-43, 45, and 47, or fragments thereof. In some embodiments, the nucleic acid molecule of the invention comprises at least one nucleotide sequence that is substantially homologous to the nucleotide sequence selected from SEQ ID NOs: 14-24, 26, 27, 29, 30, 32, 33, 35, 36, 38, 39, 41-43, 45, and 47, or fragments thereof. For example, in certain embodiments, the nucleotide sequence has a degree of identity with respect to the original nucleotide sequence of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5% to the nucleotide sequence selected from SEQ ID NOs: 14-24, 26, 27, 29, 30, 32, 33, 35, 36, 38, 39, 41-43, 45, and 47 or fragments thereof.

The isolated nucleic acid sequence encoding a peptide of the invention can be obtained using any of the many recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned.

The isolated nucleic acid may comprise any type of nucleic acid, including, but not limited to DNA and RNA. For example, in one embodiment, the composition comprises an isolated DNA molecule, including for example, an isolated cDNA molecule, encoding a polypeptide of the invention, or functional fragment thereof. In one embodiment, the composition comprises an isolated RNA molecule encoding the polypeptide of the invention, or a functional fragment thereof.

The nucleic acid molecules of the present invention can be modified to improve stability in serum or in growth medium for cell cultures. Modifications can be added to enhance stability, functionality, and/or specificity and to minimize immunostimulatory properties of the nucleic acid molecule of the invention. For example, in order to enhance the stability, the 3′-residues may be stabilized against degradation, e.g., they may be selected such that they consist of purine nucleotides, particularly adenosine or guanosine nucleotides. Alternatively, substitution of pyrimidine nucleotides by modified analogues, e.g., substitution of uridine by 2′-deoxythymidine is tolerated and does not affect function of the molecule.

In one embodiment of the present invention the nucleic acid molecule may contain at least one modified nucleotide analogue. For example, the ends may be stabilized by incorporating modified nucleotide analogues.

Non-limiting examples of nucleotide analogues include sugar- and/or backbone-modified ribonucleotides (i.e., include modifications to the phosphate-sugar backbone). For example, the phosphodiester linkages of natural RNA may be modified to include at least one of a nitrogen or sulfur heteroatom. In one embodiment of the backbone-modified ribonucleotides, the phosphoester group connecting to adjacent ribonucleotides is replaced by a modified group, e.g., of phosphothioate group. In some embodiments of sugar-modified ribonucleotides, the 2′ OH-group is replaced by a group selected from H, OR, R, halo, SH, SR, NH₂, NHR, NR₂ or ON, wherein R is C₁-C₆ alkyl, alkenyl or alkynyl and halo is F, Cl, Br or I.

Other examples of modifications are nucleobase-modified ribonucleotides, i.e., ribonucleotides, containing at least one non-naturally occurring nucleobase instead of a naturally occurring nucleobase. Bases may be modified to block the activity of adenosine deaminase. Exemplary modified nucleobases include, but are not limited to, uridine and/or cytidine modified at the 5-position, e.g., 5-(2-amino)propyl uridine, 5-bromo uridine; adenosine and/or guanosines modified at the 8 position, e.g., 8-bromo guanosine; deaza nucleotides, e.g., 7-deaza-adenosine; O- and N-alkylated nucleotides, e.g., N6-methyl adenosine are suitable. It should be noted that the above modifications may be combined.

In some instances, the nucleic acid molecule comprises at least one of the following chemical modifications: 2′-H, 2′-O-methyl, or 2′-OH modification of one or more nucleotides. In certain embodiments, a nucleic acid molecule of the invention can have enhanced resistance to nucleases. For increased nuclease resistance, a nucleic acid molecule, can include, for example, 2′-modified ribose units and/or phosphorothioate linkages. For example, the 2′ hydroxyl group (OH) can be modified or replaced with a number of different “oxy” or “deoxy” substituents. For increased nuclease resistance the nucleic acid molecules of the invention can include 2′-O-methyl, 2′-fluorine, 2′-O-methoxyethyl, 2′-O-aminopropyl, 2′-amino, and/or phosphorothioate linkages. Inclusion of locked nucleic acids (LNA), ethylene nucleic acids (ENA), e.g., 2′-4′-ethylene-bridged nucleic acids, and certain nucleobase modifications such as 2-amino-A, 2-thio (e.g., 2-thio-U), G-clamp modifications, can also increase binding affinity to a target.

In one embodiment, the nucleic acid molecule includes a 2′-modified nucleotide, e.g., a 2′-deoxy, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA). In one embodiment, the nucleic acid molecule includes at least one 2′-O-methyl-modified nucleotide, and in some embodiments, all of the nucleotides of the nucleic acid molecule include a 2′-O-methyl modification.

The present invention also includes a vector in which the isolated nucleic acid of the present invention is inserted. The art is replete with suitable vectors that are useful in the present invention.

In brief summary, the expression of natural or synthetic nucleic acids encoding a peptide of the invention is typically achieved by operably linking a nucleic acid encoding the peptide or portions thereof to a promoter, and incorporating the construct into an expression vector. The vectors to be used are suitable for replication and, optionally, integration in eukaryotic cells. Typical vectors contain transcription and translation terminators, initiation sequences, and promoters useful for regulation of the expression of the desired nucleic acid sequence.

The vectors of the present invention may also be used for nucleic acid immunization and gene therapy, using standard gene delivery protocols. Methods for gene delivery are known in the art. See, e.g., U.S. Pat. Nos. 5,399,346, 5,580,859, 5,589,466, incorporated by reference herein in their entireties. In another embodiment, the invention provides a gene therapy vector.

The isolated nucleic acid of the invention can be cloned into a number of types of vectors. For example, the nucleic acid can be cloned into a vector including, but not limited to a plasmid, a phagemid, a phage derivative, an animal virus, and a cosmid. Vectors of interest include expression vectors, replication vectors, probe generation vectors, and sequencing vectors.

Further, the vector may be provided to a cell in the form of a viral vector. Viral vector technology is well known in the art and is described, for example, in Sambrook et al. (2012, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In one embodiment, lentivirus vectors are used.

For example, vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity. In one embodiment, the composition includes a vector derived from an adeno-associated virus (AAV). Adeno-associated viral (AAV) vectors have become powerful gene delivery tools for the treatment of various disorders. AAV vectors possess a number of features that render them ideally suited for gene therapy, including a lack of pathogenicity, minimal immunogenicity, and the ability to transduce postmitotic cells in a stable and efficient manner. Expression of a particular gene contained within an AAV vector can be specifically targeted to one or more types of cells by choosing the appropriate combination of AAV serotype, promoter, and delivery method

In certain embodiments, the vector also includes conventional control elements which are operably linked to the transgene in a manner which permits its transcription, translation and/or expression in a cell transfected with the plasmid vector or infected with the virus produced by the invention. As used herein, “operably linked” sequences include both expression control sequences that are contiguous with the gene of interest and expression control sequences that act in trans or at a distance to control the gene of interest. Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A great number of expression control sequences, including promoters which are native, constitutive, inducible and/or tissue-specific, are known in the art and may be utilized.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1α (EF-1α). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

Enhancer sequences found on a vector also regulates expression of the gene contained therein. Typically, enhancers are bound with protein factors to enhance the transcription of a gene. Enhancers may be located upstream or downstream of the gene it regulates. Enhancers may also be tissue-specific to enhance transcription in a specific cell or tissue type. In one embodiment, the vector of the present invention comprises one or more enhancers to boost transcription of the gene present within the vector.

In one embodiment, the isolated nucleic acid encoding a peptide of the invention comprises in vitro transcribed (IVT) RNA. The RNA is produced by in vitro transcription using a polymerase chain reaction (PCR)-generated template. DNA of interest from any source can be directly converted by PCR into a template for in vitro mRNA synthesis using appropriate primers and RNA polymerase. The source of the DNA can be, for example, genomic DNA, plasmid DNA, phage DNA, cDNA, synthetic DNA sequence or any other appropriate source of DNA.

In one embodiment, the DNA to be used for PCR contains an open reading frame. The DNA can be from a naturally occurring DNA sequence from the genome of an organism. In one embodiment, the DNA is a full length gene of interest of a portion of a gene. The gene can include some or all of the 5′ and/or 3′ untranslated regions (UTRs). The gene can include exons and introns. In one embodiment, the DNA to be used for PCR is a human gene. In another embodiment, the DNA to be used for PCR is a human gene including the 5′ and 3′ UTRs. The DNA can alternatively be an artificial DNA sequence that is not normally expressed in a naturally occurring organism. An exemplary artificial DNA sequence is one that contains portions of genes that are ligated together to form an open reading frame that encodes a fusion protein. The portions of DNA that are ligated together can be from a single organism or from more than one organism.

Genes that can be used as sources of DNA for PCR include genes that encode polypeptides that provide a therapeutic or prophylactic effect to an organism or that can be used to diagnose a disease or disorder in an organism. Examples of such genes are genes which are useful for a short term treatment, or where there are safety concerns regarding dosage or the expressed gene. For example, for treatment of cancer, autoimmune disorders, parasitic, viral, bacterial, fungal or other infections, the transgene(s) to be expressed may encode a polypeptide that functions as a ligand or receptor for cells of the immune system, or can function to stimulate or inhibit the immune system of an organism. In some embodiments, it is not desirable to have prolonged ongoing stimulation of the immune system, nor necessary to produce changes which last after successful treatment, since this may then elicit anew problem. For treatment of an autoimmune disorder, it may be desirable to inhibit or suppress the immune system during a flare-up, but not long term, which could result in the patient becoming overly sensitive to an infection.

PCR is used to generate a template for in vitro transcription of mRNA which is used for transfection. Methods for performing PCR are well known in the art. Primers for use in PCR are designed to have regions that are substantially complementary to regions of the DNA to be used as a template for the PCR. “Substantially complementary”, as used herein, refers to sequences of nucleotides where a majority or all of the bases in the primer sequence are complementary, or one or more bases are non-complementary, or mismatched. Substantially complementary sequences are able to anneal or hybridize with the intended DNA target under annealing conditions used for PCR. The primers can be designed to be substantially complementary to any portion of the DNA template. For example, the primers can be designed to amplify the portion of a gene that is normally transcribed in cells (the open reading frame), including 5′ and 3′ UTRs. The primers can also be designed to amplify a portion of a gene that encodes a particular domain of interest. In one embodiment, the primers are designed to amplify the coding region of a human cDNA, including all or portions of the 5′ and 3′ UTRs. Primers useful for PCR are generated by synthetic methods that are well known in the art. “Forward primers” are primers that contain a region of nucleotides that are substantially complementary to nucleotides on the DNA template that are upstream of the DNA sequence that is to be amplified. “Upstream” is used herein to refer to a location 5, to the DNA sequence to be amplified relative to the coding strand. “Reverse primers” are primers that contain a region of nucleotides that are substantially complementary to a double-stranded DNA template that are downstream of the DNA sequence that is to be amplified. “Downstream” is used herein to refer to a location 3′ to the DNA sequence to be amplified relative to the coding strand.

Any DNA polymerase useful for PCR can be used in the methods disclosed herein. The reagents and polymerase are commercially available from a number of sources.

Chemical structures with the ability to promote stability and/or translation efficiency may also be used. In one embodiment, the RNA has 5′ and 3′ UTRs. In one embodiment, the 5′ UTR is between zero and 3000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths required to achieve optimal translation efficiency following transfection of the transcribed RNA.

The 5′ and 3′ UTRs can be the naturally occurring, endogenous 5′ and 3′ UTRs for the gene of interest. Alternatively, UTR sequences that are not endogenous to the gene of interest can be added by incorporating the UTR sequences into the forward and reverse primers or by any other modifications of the template. The use of UTR sequences that are not endogenous to the gene of interest can be useful for modifying the stability and/or translation efficiency of the RNA. For example, it is known that AU-rich elements in 3′ UTR sequences can decrease the stability of mRNA. Therefore, 3′ UTRs can be selected or designed to increase the stability of the transcribed RNA based on properties of UTRs that are well known in the art.

In one embodiment, the 5′ UTR can contain the Kozak sequence of the endogenous gene. Alternatively, when a 5′ UTR that is not endogenous to the gene of interest is being added by PCR as described above, a consensus Kozak sequence can be redesigned by adding the 5′ UTR sequence. Kozak sequences can increase the efficiency of translation of some RNA transcripts, but does not appear to be required for all RNAs to enable efficient translation. The requirement for Kozak sequences for many mRNAs is known in the art. In other embodiments the 5′ UTR can be derived from an RNA virus whose RNA genome is stable in cells. In other embodiments various nucleotide analogues can be used in the 3′ or 5′ UTR to impede exonuclease degradation of the mRNA.

To enable synthesis of RNA from a DNA template without the need for gene cloning, a promoter of transcription should be attached to the DNA template upstream of the sequence to be transcribed. When a sequence that functions as a promoter for an RNA polymerase is added to the 5′ end of the forward primer, the RNA polymerase promoter becomes incorporated into the PCR product upstream of the open reading frame that is to be transcribed. In one embodiment, the promoter is a T7 polymerase promoter, as described elsewhere herein. Other useful promoters include, but are not limited to, T3 and SP6 RNA polymerase promoters. Consensus nucleotide sequences for T7, T3 and SP6 promoters are known in the art.

In one embodiment, the mRNA has both a cap on the 5′ end and a 3′ poly(A) tail which determine ribosome binding, initiation of translation and stability mRNA in the cell. On a circular DNA template, for instance, plasmid DNA, RNA polymerase produces a long concatameric product which is not suitable for expression in eukaryotic cells. The transcription of plasmid DNA linearized at the end of the 3′ UTR results in normal sized mRNA which is not effective in eukaryotic transfection even if it is after transcription.

On a linear DNA template, phage T7 RNA polymerase can extend the 3′ end of the transcript beyond the last base of the template (Schenbom and Mierendorf, Nuc Acids Res., 13:6223-36 (1985); Nacheva and Berzal-Herranz, Eur. J. Biochem., 270:1485-65 (2003).

The conventional method of integration of polyA/T stretches into a DNA template is molecular cloning. However polyA/T sequence integrated into plasmid DNA can cause plasmid instability, which is why plasmid DNA templates obtained from bacterial cells are often highly contaminated with deletions and other aberrations. This makes cloning procedures not only laborious and time consuming but often not reliable. That is why a method which allows construction of DNA templates with polyA/T 3′ stretch without cloning highly desirable.

The polyA/T segment of the transcriptional DNA template can be produced during PCR by using a reverse primer containing a polyT tail, such as 100T tail (size can be 50-5000 T), or after PCR by any other method, including, but not limited to, DNA ligation or in vitro recombination. Poly(A) tails also provide stability to RNAs and reduce their degradation. Generally, the length of a poly(A) tail positively correlates with the stability of the transcribed RNA. In one embodiment, the poly(A) tail is between 100 and 5000 adenosines.

Poly(A) tails of RNAs can be further extended following in vitro transcription with the use of a poly(A) polymerase, such as E. coli polyA polymerase (E-PAP). In one embodiment, increasing the length of a poly(A) tail from 100 nucleotides to between 300 and 400 nucleotides results in about a two-fold increase in the translation efficiency of the RNA. Additionally, the attachment of different chemical groups to the 3′ end can increase mRNA stability. Such attachment can contain modified/artificial nucleotides, aptamers and other compounds. For example, ATP analogs can be incorporated into the poly(A) tail using poly(A) polymerase. ATP analogs can further increase the stability of the RNA.

5′ caps on also provide stability to RNA molecules. In one embodiment, RNAs produced by the methods disclosed herein include a 5′ cap. The 5′ cap is provided using techniques known in the art and described herein (Cougot, et al., Trends in Biochem. Sci., 29:436-444 (2001); Stepinski, et al., RNA, 7:1468-95 (2001); Elango, et al., Biochim. Biophys. Res. Commun., 330:958-966 (2005)).

The RNAs produced by the methods disclosed herein can also contain an internal ribosome entry site (IRES) sequence. The IRES sequence may be any viral, chromosomal or artificially designed sequence which initiates cap-independent ribosome binding to mRNA and facilitates the initiation of translation. Any solutes suitable for cell electroporation, which can contain factors facilitating cellular permeability and viability such as sugars, peptides, lipids, proteins, antioxidants, and surfactants can be included.

In one aspect, the present invention relates, in part to a composition comprising at least one peptide of the present invention and/or at least one nucleic acid molecule of the present invention. For example, in certain aspects, the composition comprises DNA, RNA, mRNA, or cDNA encoding one or more peptides described herein. In some embodiments, the composition reduces the clearance of at least one cell of interest.

In some embodiments, the composition increases the persistence of at least one cell of interest. Examples of such cells include, but are not limited to: donor cells, T cells, CAR cells, CAR T cells, engineered TCR-expressing T cell, etc.

In some embodiments, the composition inhibits or reduces the level of at least one NK cell, alloresponsive cell, immune cell, T cell, B cell, NK cell, white blood cell, myeloid cell, or plasma cell associated with a donor cell. In some embodiments, the composition inhibits an NK cell-mediated killing of at least one donor cell.

In some embodiments, the composition prevents, reduces, or inhibits an immune response against at least one donor cell.

It is understood that the composition of the present invention may be made by a method that is well known in the art, including but not limited to chemical synthesis by solid phase synthesis and purification away from the other products of the chemical reactions by HPLC, or production by the expression of a nucleic acid sequence (e.g., a DNA sequence) encoding the peptide of the present invention in an in vitro translation system or in a living cell. In addition, the composition can comprise a cellular component isolated from a biological sample. The composition isolated and extensively dialyzed to remove one or more undesired small molecular weight molecules and/or lyophilized for more ready formulation into a desired vehicle. A peptide sequence may be synthesized by methods known to those of ordinary skill in the art, such as, for example, peptide synthesis using automated peptide synthesis machines, such as those available from Applied Biosystems, Inc., Foster City, CA (Foster City, CA).

Longer peptides or polypeptides may be also prepared, e.g., by recombinant means. In certain embodiments, a nucleic acid encoding the peptide described herein may be used, for example, to produce a composition in vitro or in vivo for the various compositions and methods of the present invention. For example, in certain embodiments, the nucleic acid encoding the peptide of the present invention is comprised in, for example, a vector in a recombinant cell. The nucleic acid may be expressed to produce a peptide or polypeptide comprising an antigenic sequence. The peptide or polypeptide may be secreted from the cell, or comprised as part of or within the cell.

### Modified Cells

In various aspects, the present invention provides a genetically engineered cell that comprises at least one peptide and/or at least one nucleic acid molecule of the present invention. In some embodiments, the cell is genetically modified to comprise or express the peptide of the invention. In some embodiments, the cell is genetically modified to not express a beta-2-microglobulin (B2M), class II major histocompatibility complex transactivator (CIITA), and/or native T cell receptor (TCR). In certain embodiments, the cell is genetically modified to reduce or eliminate an MHC I, MHC II, and/or native TCR ab heterodimer. For example, in some embodiments, the cell is triple knockout (TKO) cell that does not express an MHC I, MHC II, and/or native TCR.

The TKO cells of the present invention can be prepared using any method known in the art. For example, in some embodiments, efficient production of TKO cells can be facilitated by genome editing techniques, such as protein-based techniques (e.g., zinc-finger nucleases (ZFNs), transcription activator-like effector nucleases (TALENs), etc.) or RNA-based DNA recognition techniques derived from a bacterial adaptive immune system (e.g., and the clustered regularly interspaced short palindromic repeats (CRISPR)/Cas nuclease systems). In some embodiments, the TKO cell is achieved by using (i) Cas9 protein or expression vectors or RNAs encoding the Cas9 protein and (ii) target-locus-specific guide RNAs (gRNAs) or expression vectors encoding the gRNA. In some embodiments, the TKO cell is achieved by using a method for specifically cutting a target sequence by introducing, into a cell, (i) guide RNAs designed based on the target sequence and (ii) RNAs encoding a Cas protein (e.g., PCT International Publication, No. WO/2014/093661, which is incorporated by reference herein in its entirety).

In some embodiments, a method for knocking out a target gene in a cell is a method including the step of introducing a CRISPR-Cas system into a cell having one or more kinds of target genes, the CRISPR-Cas system being able to produce (i) three or more kinds of guide RNAs for each of the one or more kinds of target genes and (ii) a Cas protein. In an embodiment of the present invention, the one or more kinds of target genes are knocked out by (i) causing three or more kinds of guide RNAs to target each of the one or more kinds of target genes and then (ii) causing a Cas protein to cut each of the one or more kinds of target gene.

CRISPR includes several tens of base pairs of short repetitive sequence and is a locus serving as a type of acquired immune system in a prokaryote. It is known that a CRISPR-associated (cas) gene cluster encoding nuclease and helicase exists in the vicinity of a CRISPR repetitive sequence. Foreign DNA is fragmented, by protein encoded by any cas gene cluster, into a length of approximately 30 base pairs. In a case where it is inserted into the CRISPR locus by any method, the fragments function as immunological memory. At the CRISPR locus, RNA is transcribed, so that RNA is fragmented by Cas protein into smaller RNAs (crRNAs) having respective foreign sequences. The RNA guides another Cas protein to the foreign DNA (or RNA derived from the foreign DNA), so that a mechanism similar to RNAi of a eukaryote suppresses the function of the foreign DNA (or RNA derived from the foreign DNA). The CRISPR-Cas system is applied to RNA-guided genomic engineering at a cellular level or an individual level.

There are type I, type II, and type III of CRISPR/Cas. The type mainly used in genome editing is type II CRISPR/Cas. In the type II, Cas9 is used as RGN. Cas9 of Streptococcus pyogenes recognizes three bases, NGG, as Proto-spacer Adjacent Motif (PAM). Therefore, a sequence in which two guanines are adjacent can be cut at the upstream thereof. This makes it possible to target any one of substantially all of DNA sequences on a genome.

A method in which CRISPR/Cas is used allows, only by synthesis of short gRNA homologous with a target DNA sequence as described above, editing of a genome with use of Cas protein which is a single protein. Examples of the Cas protein encompass, but are not limited to, CAS1, CAS1B, CAS2, CAS3, CAS4, Cas5, Cas6, Cas7, Cas8, Cas9, Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, a homologue thereof, and a mutant thereof. These enzymes are publicly known. For example, in one embodiment, the Cas protein is a Cas9 protein.

Examples of a manner in which a Cas protein can be produced encompass (i) a form in which Cas protein itself is introduced as a protein into a cell, (ii) a form in which RNA encoding a Cas protein is introduced into a cell, and (iii) a form in which a vector (such as DNA vector) that can express a Cas protein in a cell is introduced into a cell. In some embodiments where a vector that can express a Cas protein in a cell is used, an expression vector that contains (i) a DNA encoding the Cas protein and (ii) an expression regulatory sequence (such as a promoter) upstream of the DNA is used.

Components constituting the CRISPR-Cas system are not limited to any particular ones, provided that guide RNAs and Cas protein can be produced in a cell. Examples of a manner in which guide RNA can be produced encompass (i) a form in which a guide RNA itself is introduced as RNA into a cell and (ii) a form in which a vector (such as DNA vector) that can express a guide RNA in a cell is introduced into a cell. In a case where a guide RNA itself is introduced into a cell, for example, a guide RNA can be obtained by chemical synthesis or in vitro transcription of a guide RNA. In some embodiments where a vector that can express a guide RNA in a cell is used, an expression vector that contains (i) a DNA encoding the guide RNA and (ii) an expression regulatory sequence (such as a promoter) upstream of the DNA is used.

A vector can be a nucleic acid molecule containing DNA, RNA, or both of DNA and RNA. Specific examples of the vector encompass, but are not limited to, plasmid vectors and virus vectors (such as a retrovirus vector, an adenovirus vector, and an adeno-associated virus vector). The vector can be a vector which is autonomously replicated in a host cell into which the vector is introduced. Alternatively, the vector can be a vector that is integrated into a genome of a host cell when the vector is introduced into the host cell.

The kind of an expression regulatory sequence to be used in a vector expressing a guide RNA or a Cas protein is not particularly limited. Any expression regulatory sequence functions in a cell into which an expression vector is introduced can be used. Examples of the expression regulatory sequence encompass, but are not limited to, promoters, enhancers, internal ribosome entry sites (IRES), and any other expression regulatory elements (e.g. transcription termination signals such as a polyadenylation signal and a poly(U) sequence). The expression regulatory sequence can be a sequence that induces constitutive expression of genes in a wide range of host cells, or can be a sequence that induces expression of genes in certain host cells. Examples of a tissue-specific promoter that induces expression of gene in only a certain host cell encompass promoters which can induce expression in a desired tissue such as muscle, nerve, bone, skin, blood, certain organs (e.g. liver and pancreas), and certain cell types (e.g. lymphocyte). Specific examples of the promoter encompass, but are not limited to, pol III promoter, pol II promoter, pol I promoter, and a combination thereof. Specific examples of the pol III promoter encompass, but are not limited to, U6 promoter and HI promoter. Examples of the poll promoter encompass, but are not limited to, retrovirus Rous sarcoma VIMS (RSV) LTR promoters, cytomegalovirus (CMV) promoters), SV40 promoters, dihydrofolate reductase promoters, p actin promoters, phospho-glycerol kinase (PGR) promoters, and EFL promoters.

In a case where an expression vector expressing guide RNA or Cas protein is to be used, at least four kinds of DNAs that encode three or more kinds of guide RNAs and a Cas protein can be contained in a single expression vector or contained in respective expression vectors. For example, the following cases are possible:
(1) A case where a single expression vector, which contains all of DNAs encoding three or more kinds of guide RNAs and a Cas protein, is to be used;
(2) A case where an expression vector, which contains all of DNAs encoding three or more kinds of guide RNAs, and an expression vector encoding a Cas protein, are to be used;
(3) A case where three or more kinds of expression vectors, which contain respective three or more kinds of guide RNAs, and an expression vector, which contains DNA encoding a Cas protein, are to be used.

The type of cell into which the CRISPR-Cas system in accordance with an embodiment of the present invention is to be introduced is not limited to any particular one. The cell can be a prokaryotic cell or a eukaryotic cell. In one embodiment, the cell is a eukaryotic cell. In another embodiment, the cell is an animal cell. In one embodiment, the animal cell is a mammal cell (such as a mouse cell or a human cell).

The CRISPR-Cas system, when introduced into a cell, is not particularly limited in terms of which site of the cell the CRISPR-Cas system is to be introduced. The CRISPR-Cas system can be introduced into a nucleus, or can be introduced into a cytoplasm. In a case where the CRISPR-Cas system is to be introduced in a form of RNA, the CRISPR-Cas system can be introduced into a cytoplasm.

The CRISPR-Cas system can be introduced into a cell by a method such as viral particles, liposome, electroporation, microinjection, and conjugation.

The method of the present invention for knocking out a target gene in a cell can be used for production of a knockout non-human organism, gene therapy, drug screening, and diagnosis and prognosis of a disease. In some embodiments, the method is used for production of a knockout non-human organism.

In some embodiments, the nucleic acid sequence is delivered into cells using a retroviral or lentiviral vector. For example, retroviral and lentiviral vectors expressing a peptide of the invention can be delivered into different types of eukaryotic cells as well as into tissues and whole organisms using transduced cells as carriers or cell-free local or systemic delivery of encapsulated, bound or naked vectors. The method used can be for any purpose where stable expression is required or sufficient.

In other embodiments, the nucleic acid sequence is delivered into cells using in vitro transcribed mRNA. In vitro transcribed mRNA can be delivered into different types of eukaryotic cells as well as into tissues and whole organisms using transfected cells as carriers or cell-free local or systemic delivery of encapsulated, bound or naked mRNA. The method used can be for any purpose where transient expression is required or sufficient.

In certain embodiments, the cell may be of any suitable cell type that can express the desired peptide. In certain embodiments, the modified cell is used in a method where the cell is introduced into a recipient. In certain embodiments, the cell is autologous, allogeneic, syngeneic or xenogeneic with respect to recipient. In certain embodiments the cell is derived from a stem cell or precursor cell. In some embodiments, the stem cell or precursor cell from which the modified cell is derived is autologous, allogeneic, syngeneic or xenogeneic with respect to recipient.

In one embodiment, the cell is an immune cell. Exemplary immune cells include, but are not limited to, T cells (including killer T cells, helper T cells, regulatory T cells, T cell bearing TCRs, alloresponsive T cell, allo-specific T cell, T cell bearing alloreactive TCR, and gamma delta T cells), B cells, antigen presenting cells (APCs), NK cells, NK T cells, CAR T cells, and TCR-expressing T cells.

For example, in certain embodiments, the composition comprises an immune cell that comprises or expresses one or more peptide (e.g., SCT) described herein. Exemplary immune cells that may comprise or express one or more CARs or engineered TCRs described herein include, but are not limited to, T cells (including killer T cells, helper T cells, regulatory T cells, and gamma delta T cells), natural killer (NK) cells, NK T cells, CAR T cells, and engineered TCR-expressing T cells. Exemplary immune cells that may comprise or express one or more peptide (e.g., SCT) described herein include, but are not limited to, an antigen presenting cell, dendritic cell, B cell, macrophage, Langerhans cell, T cell, NK cell, NK T cell, CAR T cells, and engineered TCR-expressing T cells.

In one embodiment, the cell is an antigen presenting cell (APC). For example, in certain embodiments, the composition comprises an APC that is modified to comprise or express one or more peptide (e.g., SCT) described herein. Exemplary APCs include, but is not limited to, dendritic cells (DCs), macrophages, Langerhans cells, B cells, and the like.

The disclosed compositions and methods can be applied to the modulation of T cell activity in basic research and therapy, in the fields of cancer, stem cells, acute and chronic infections, and autoimmune diseases, including the assessment of the ability of the genetically modified T cell to kill a target cancer cell.

Prior to expansion and genetic modification of the T cells of the invention, a source of T cells is obtained from a subject. T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In certain embodiments of the present invention, any number of T cell lines available in the art, may be used. In certain embodiments of the present invention, T cells can be obtained from a unit of blood collected from a subject using any number of techniques known to the skilled artisan, such as Ficoll™ separation. In one embodiment, cells from the circulating blood of an individual are obtained by apheresis. The apheresis product typically contains lymphocytes, including T cells, monocytes, granulocytes, B cells, other nucleated white blood cells, red blood cells, and platelets. In one embodiment, the cells collected by apheresis may be washed to remove the plasma fraction and to place the cells in an appropriate buffer or media for subsequent processing steps. In one embodiment of the invention, the cells are washed with phosphate buffered saline (PBS). In an alternative embodiment, the wash solution lacks calcium and may lack magnesium or may lack many if not all divalent cations. Again, surprisingly, initial activation steps in the absence of calcium lead to magnified activation. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca²⁺-free, Mg²⁺-free PBS, PlasmaLyte A, or other saline solution with or without buffer. Alternatively, the undesirable components of the apheresis sample may be removed and the cells directly resuspended in culture media.

In another embodiment, T cells are isolated from peripheral blood lymphocytes by lysing the red blood cells and depleting the monocytes, for example, by centrifugation through a PERCOLL™ gradient or by counterflow centrifugal elutriation. A specific subpopulation of T cells, such as CD3⁺, CD28⁺, CD4⁺, CD8⁺, CD45RA⁺, and CD45RO⁺T cells, can be further isolated by positive or negative selection techniques. For example, in one embodiment, T cells are isolated by incubation with anti-CD3/anti-CD28 (i.e., 3×28)-conjugated beads, such as DYNABEADS® M-450 CD3/CD28 T, for a time period sufficient for positive selection of the desired T cells. In one embodiment, the time period is about 30 minutes. In a further embodiment, the time period ranges from 30 minutes to 36 hours or longer and all integer values there between. In a further embodiment, the time period is at least 1, 2, 3, 4, 5, or 6 hours. In yet another embodiment, the time period is 10 to 24 hours. In one embodiment, the incubation time period is 24 hours. For isolation of T cells from patients with leukemia, use of longer incubation times, such as 24 hours, can increase cell yield. Longer incubation times may be used to isolate T cells in any situation where there are few T cells as compared to other cell types, such in isolating tumor infiltrating lymphocytes (TIL) from tumor tissue or from immune-compromised individuals. Further, use of longer incubation times can increase the efficiency of capture of CD8+ T cells. Thus, by simply shortening or lengthening the time T cells are allowed to bind to the CD3/CD28 beads and/or by increasing or decreasing the ratio of beads to T cells (as described further herein), subpopulations of T cells can be selected for or against at culture initiation or at other time points during the process. Additionally, by increasing or decreasing the ratio of anti-CD3 and/or anti-CD28 antibodies on the beads or other surface, subpopulations of T cells can be selected for or against at culture initiation or at other desired time points. The skilled artisan would recognize that multiple rounds of selection can also be used in the context of this invention. In certain embodiments, it may be desirable to perform the selection procedure and use the “unselected” cells in the activation and expansion process. “Unselected” cells can also be subjected to further rounds of selection.

Enrichment of a T cell population by negative selection can be accomplished with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4⁺ cells by negative selection, a monoclonal antibody cocktail typically includes antibodies to CD14, CD20, CD11b, CD16, HLA-DR, and CD8. In certain embodiments, it may be desirable to enrich for or positively select for regulatory T cells which typically express CD4⁺, CD25⁺, CD62L^{hi}, GITR⁺, and FoxP3⁺. Alternatively, in certain embodiments, T regulatory cells are depleted by anti-C25 conjugated beads or other similar method of selection.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (i.e., leukemic blood, tumor tissue, etc.). Such populations of cells may have therapeutic value and would be desirable to obtain. For example, using high concentration of cells allows more efficient selection of CD8¹ T cells that normally have weaker CD28 expression.

In a related embodiment, it may be desirable to use lower concentrations of cells. By significantly diluting the mixture of T cells and surface (e.g., particles such as beads), interactions between the particles and cells is minimized. This selects for cells that express high amounts of desired antigens to be bound to the particles. For example, CD4⁺ T cells express higher levels of CD28 and are more efficiently captured than CD8⁺ T cells in dilute concentrations. In one embodiment, the concentration of cells used is 5×10⁶/ml. In other embodiments, the concentration used can be from about 1×10⁵/ml to 1×10⁶/ml, and any integer value in between.

In other embodiments, the cells may be incubated on a rotator for varying lengths of time at varying speeds at either 2-10° C. or at room temperature.

T cells for stimulation can also be frozen after a washing step. Wishing not to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, one method involves using PBS containing 20% DMSO and 8% human serum albumin, or culture media containing 10% Dextran 40 and 5% Dextrose, 20% Human Serum Albumin and 7.5% DMSO, or 31.25% Plasmalyte-A, 31.25% Dextrose 5%, 0.45% NaCl, 10% Dextran 40 and 5% Dextrose, 20% Human Serum Albumin, and 7.5% DMSO or other suitable cell freezing media containing for example, Hespan and PlasmaLyte A, the cells then are frozen to −80° C. at a rate of 1° C. per minute and stored in the vapor phase of a liquid nitrogen storage tank. Other methods of controlled freezing may be used as well as uncontrolled freezing immediately at −20° C. or in liquid nitrogen.

In certain embodiments, cryopreserved cells are thawed and washed as described herein and allowed to rest for one hour at room temperature prior to activation using the methods of the present invention.

Also contemplated in the context of the invention is the collection of blood samples or apheresis product from a subject at a time period prior to when the expanded cells as described herein might be needed. As such, the source of the cells to be expanded can be collected at any time point necessary, and desired cells, such as T cells, isolated and frozen for later use in T cell therapy for any number of diseases or conditions that would benefit from T cell therapy, such as those described herein. In one embodiment a blood sample or an apheresis is taken from a generally healthy subject. In certain embodiments, a blood sample or an apheresis is taken from a generally healthy subject who is at risk of developing a disease, but who has not yet developed a disease, and the cells of interest are isolated and frozen for later use. In certain embodiments, the T cells may be expanded, frozen, and used at a later time. In certain embodiments, samples are collected from a patient shortly after diagnosis of a particular disease as described herein but prior to any treatments. In a further embodiment, the cells are isolated from a blood sample or an apheresis from a subject prior to any number of relevant treatment modalities, including but not limited to treatment with agents such as natalizumab, efalizumab, antiviral agents, chemotherapy, radiation, immunosuppressive agents, such as cyclosporin, azathioprine, methotrexate, mycophenolate, and FK506, antibodies, or other immunoablative agents such as CAMPATH, anti-CD3 antibodies, cytoxan, fludarabine, cyclosporin, FK506, rapamycin, mycophenolic acid, steroids, FR901228, and irradiation. These drugs inhibit either the calcium dependent phosphatase calcineurin (cyclosporine and FK506) or inhibit the p70S6 kinase that is important for growth factor induced signaling (rapamycin) (Liu et al., Cell 66:807-815, 1991; Henderson et al., Immun. 73:316-321, 1991; Bierer et al., Curr. Opin. Immun. 5:763-773, 1993). In a further embodiment, the cells are isolated for a patient and frozen for later use in conjunction with (e.g., before, simultaneously or following) bone marrow or stem cell transplantation, T cell ablative therapy using either chemotherapy agents such as, fludarabine, external-beam radiation therapy (XRT), cyclophosphamide, or antibodies such as OKT3 or CAMPATH. In another embodiment, the cells are isolated prior to and can be frozen for later use for treatment following B-cell ablative therapy such as agents that react with CD20, e.g., Rituxan.

In a further embodiment of the present invention, T cells are obtained from a patient directly following treatment. In this regard, it has been observed that following certain cancer treatments, in particular treatments with drugs that damage the immune system, shortly after treatment during the period when patients would normally be recovering from the treatment, the quality of T cells obtained may be optimal or improved for their ability to expand ex vivo. Likewise, following ex vivo manipulation using the methods described herein, these cells may be in a state for enhanced engraftment and in vivo expansion. Thus, it is contemplated within the context of the present invention to collect blood cells, including T cells, dendritic cells, or other cells of the hematopoietic lineage, during this recovery phase. Further, in certain embodiments, mobilization (for example, mobilization with GM-CSF) and conditioning regimens can be used to create a condition in a subject wherein repopulation, recirculation, regeneration, and/or expansion of particular cell types is favored, especially during a defined window of time following therapy. Illustrative cell types include T cells, B cells, dendritic cells, and other cells of the immune system.

Whether prior to or after genetic modification of the T cells to express a peptide of the invention, the T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005.

Generally, the T cells of the invention are expanded by contact with a surface having attached thereto an agent that stimulates a CD3/TCR complex associated signal and a ligand that stimulates a co-stimulatory molecule on the surface of the T cells. In some embodiments, T cell populations may be stimulated as described herein, such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. To stimulate proliferation of either CD4⁺ T cells or CD8⁺ T cells, an anti-CD3 antibody and an anti-CD28 antibody. Examples of an anti-CD28 antibody include 9.3, B-T3, XR-CD28 (Diaclone, Besangon, France) can be used as can other methods commonly known in the art (Berg et al., Transplant Proc. 30(8):3975-3977, 1998; Haanen et al., J. Exp. Med. 190(9):13191328, 1999; Garland et al., J. Immunol Meth. 227(1-2):53-63, 1999).

In certain embodiments, the primary stimulatory signal and the co-stimulatory signal for the T cell may be provided by different protocols. For example, the agents providing each signal may be in solution or coupled to a surface. When coupled to a surface, the agents may be coupled to the same surface (i.e., in “cis” formation) or to separate surfaces (i.e., in “trans” formation). Alternatively, one agent may be coupled to a surface and the other agent in solution. In one embodiment, the agent providing the co-stimulatory signal is bound to a cell surface and the agent providing the primary activation signal is in solution or coupled to a surface. In certain embodiments, both agents can be in solution. In another embodiment, the agents may be in soluble form, and then cross-linked to a surface, such as a cell expressing Fc receptors or an antibody or other binding agent which will bind to the agents. In this regard, see for example, U.S. Patent Application Publication Nos. 2004/0101519 and 2006/0034810 for artificial antigen presenting cells (aAPCs) that are contemplated for use in activating and expanding T cells in the present invention.

In one embodiment, the two agents are immobilized on beads, either on the same bead, i.e., “cis,” or to separate beads, i.e., “trans.” By way of example, the agent providing the primary activation signal is an anti-CD3 antibody or an antigen-binding fragment thereof and the agent providing the co-stimulatory signal is an anti-CD28 antibody or antigen-binding fragment thereof; and both agents are co-immobilized to the same bead in equivalent molecular amounts. In one embodiment, a 1:1 ratio of each antibody bound to the beads for CD4⁺ T cell expansion and T cell growth is used. In certain aspects of the present invention, a ratio of anti CD3:CD28 antibodies bound to the beads is used such that an increase in T cell expansion is observed as compared to the expansion observed using a ratio of 1:1. In one embodiment an increase of from about 1 to about 3 fold is observed as compared to the expansion observed using a ratio of 1:1. In one embodiment, the ratio of CD3:CD28 antibody bound to the beads ranges from 100:1 to 1:100 and all integer values there between. In one aspect of the present invention, more anti-CD28 antibody is bound to the particles than anti-CD3 antibody, i.e., the ratio of CD3:CD28 is less than one. In certain embodiments of the invention, the ratio of anti CD28 antibody to anti CD3 antibody bound to the beads is greater than 2:1. In one embodiment, a 1:100 CD3:CD28 ratio of antibody bound to beads is used. In another embodiment, a 1:75 CD3:CD28 ratio of antibody bound to beads is used. In a further embodiment, a 1:50 CD3:CD28 ratio of antibody bound to beads is used. In another embodiment, a 1:30 CD3:CD28 ratio of antibody bound to beads is used. In one embodiment, a 1:10 CD3:CD28 ratio of antibody bound to beads is used. In another embodiment, a 1:3 CD3:CD28 ratio of antibody bound to the beads is used. In yet another embodiment, a 3:1 CD3:CD28 ratio of antibody bound to the beads is used.

Ratios of particles to cells from 1:500 to 500:1 and any integer values in between may be used to stimulate T cells or other target cells. As those of ordinary skill in the art can readily appreciate, the ratio of particles to cells may depend on particle size relative to the target cell. For example, small sized beads could only bind a few cells, while larger beads could bind many. In certain embodiments the ratio of cells to particles ranges from 1:100 to 100:1 and any integer values in-between and in further embodiments the ratio comprises 1:9 to 9:1 and any integer values in between, can also be used to stimulate T cells. The ratio of anti-CD3- and anti-CD28-coupled particles to T cells that result in T cell stimulation can vary as noted above, exemplary values include 1:100, 1:50, 1:40, 1:30, 1:20, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, and 15:1 with one exemplary ratio being at least 1:1 particles per T cell. In one embodiment, a ratio of particles to cells of 1:1 or less is used. In one embodiment, a particle:cell ratio is 1:5. In further embodiments, the ratio of particles to cells can be varied depending on the day of stimulation. For example, in one embodiment, the ratio of particles to cells is from 1:1 to 10:1 on the first day and additional particles are added to the cells every day or every other day thereafter for up to 10 days, at final ratios of from 1:1 to 1:10 (based on cell counts on the day of addition). In one embodiment, the ratio of particles to cells is 1:1 on the first day of stimulation and adjusted to 1:5 on the third and fifth days of stimulation. In another embodiment, particles are added on a daily or every other day basis to a final ratio of 1:1 on the first day, and 1:5 on the third and fifth days of stimulation. In another embodiment, the ratio of particles to cells is 2:1 on the first day of stimulation and adjusted to 1:10 on the third and fifth days of stimulation. In another embodiment, particles are added on a daily or every other day basis to a final ratio of 1:1 on the first day, and 1:10 on the third and fifth days of stimulation. One of skill in the art will appreciate that a variety of other ratios may be suitable for use in the present invention. In some embodiments, ratios will vary depending on particle size and on cell size and type.

In further embodiments of the present invention, the cells, such as T cells, are combined with agent-coated beads, the beads and the cells are subsequently separated, and then the cells are cultured. In an alternative embodiment, prior to culture, the agent-coated beads and cells are not separated but are cultured together. In a further embodiment, the beads and cells are first concentrated by application of a force, such as a magnetic force, resulting in increased ligation of cell surface markers, thereby inducing cell stimulation.

By way of example, cell surface proteins may be ligated by allowing paramagnetic beads to which anti-CD3 and anti-CD28 are attached (3×28 beads) to contact the T cells. In one embodiment the cells (for example, 104 to 109 T cells) and beads (for example, DYNABEADS® M-450 CD3/CD28 T paramagnetic beads at a ratio of 1:1) are combined in a buffer, such as PBS (without divalent cations such as, calcium and magnesium). Again, those of ordinary skill in the art can readily appreciate any cell concentration may be used. For example, the target cell may be very rare in the sample and comprise only 0.01% of the sample or the entire sample (i.e., 100%) may comprise the target cell of interest. Accordingly, any cell number is within the context of the present invention. In certain embodiments, it may be desirable to significantly decrease the volume in which particles and cells are mixed together (i.e., increase the concentration of cells), to ensure maximum contact of cells and particles. For example, in one embodiment, a concentration of about 2 billion cells/ml is used. In another embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T cells. Such populations of cells may have therapeutic value and would be desirable to obtain in certain embodiments. For example, using high concentration of cells allows more efficient selection of CD8+ T cells that normally have weaker CD28 expression.

In one embodiment of the present invention, the mixture may be cultured for several hours (about 3 hours) to about 14 days or any hourly integer value in between. In another embodiment, the mixture may be cultured for 21 days. In one embodiment of the invention the beads and the T cells are cultured together for about eight days. In another embodiment, the beads and T cells are cultured together for 2-3 days. Several cycles of stimulation may also be desired such that culture time of T cells can be 60 days or more. Conditions appropriate for T cell culture include an appropriate media (e.g., Minimal Essential Media or RPMI Media 1640 or, X-vivo 15, (Lonza)) that may contain factors necessary for proliferation and viability, including serum (e.g., fetal bovine or human serum), interleukin-2 (IL-2), insulin, IFN-γ, IL-4, IL-7, GM-CSF, IL-10, IL-12, IL-15, TGFβ, and TNF-α or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, α-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO₂).

T cells that have been exposed to varied stimulation times may exhibit different characteristics. For example, typical blood or apheresed peripheral blood mononuclear cell products have a helper T cell population (T_{H}, CD4⁺) that is greater than the cytotoxic or suppressor T cell population (T_{C}, CD8¹). Ex vivo expansion of T cells by stimulating CD3 and CD28 receptors produces a population of T cells that prior to about days 8-9 consists predominately of T_{II} cells, while after about days 8-9, the population of T cells comprises an increasingly greater population of T_{C} cells. Accordingly, depending on the purpose of treatment, infusing a subject with a T cell population comprising predominately of T_{H} cells may be advantageous. Similarly, if an antigen-specific subset of T_{C} cells has been isolated it may be beneficial to expand this subset to a greater degree.

Further, in addition to CD4 and CD8 markers, other phenotypic markers vary significantly, but in large part, reproducibly during the course of the cell expansion process. Thus, such reproducibility enables the ability to tailor an activated T cell product for specific purposes.

In one embodiment, the cell comprises a chimeric antigen receptor (CAR). In certain embodiments disclosed herein, a CAR generally comprises an antigen binding domain, a transmembrane domain and an intracellular domain. In some embodiments, the CAR comprises an antigen binding domain that binds to a tumor-associated antigen or a tumor-specific antigen.

In various embodiments, the CAR can be any CAR molecule including, but not limited to, a “first generation,” “second generation,” “third generation,” “fourth generation” or “fifth generation” CAR (see, for example, Sadelain et al., Cancer Discov. 3(4):388-398 (2013); Jensen et al., Immunol. Rev. 257:127-133 (2014); Sharpe et al., Dis. Model Mech. 8(4):337-350 (2015); Brentjens et al., Clin. Cancer Res. 13:5426-5435 (2007); Gade et al., Cancer Res. 65:9080-9088 (2005); Maher et al., Nat. Biotechnol. 20:70-75 (2002); Kershaw et al., J. Immunol. 173:2143-2150 (2004); Sadelain et al., Curr. Opin. Immunol. (2009); Hollyman et al., J. Immunother. 32:169-180 (2009)).

“First generation” CARs for use in the invention comprise an antigen binding domain, for example, a single-chain variable fragment (scFv), fused to a transmembrane domain, which is fused to a cytoplasmic/intracellular domain of a TCR chain. “First generation” CARs typically have the intracellular domain from the CD3ζ-chain, which is the primary transmitter of signals from endogenous TCRs. “First generation” CARs can provide de novo antigen recognition and cause activation of both CD4+ and CD8+ T cells through their CD3ζ chain signaling domain in a single fusion molecule, independent of HLA-mediated antigen presentation.

“Second-generation” CARs for use in the invention comprise an antigen binding domain, for example, a single-chain variable fragment (scFv), fused to an intracellular signaling domain capable of activating T cells and a co-stimulatory domain designed to augment T cell potency and persistence (Sadelain et al., Cancer Discov. 3:388-398 (2013)). CAR design can therefore combine antigen recognition with signal transduction, two functions that are physiologically borne by two separate complexes, the TCR heterodimer and the CD3 complex. “Second generation” CARs include an intracellular domain from various co-stimulatory molecules, for example, CD28, 4-1BB, ICOS, OX40, and the like, in the cytoplasmic tail of the CAR to provide additional signals to the cell.

“Second generation” CARs provide both co-stimulation, for example, by CD28 or 4-1BB domains, and activation, for example, by a CD3ζ signaling domain. Preclinical studies have indicated that “Second Generation” CARs can improve the anti-tumor activity of T cells. For example, robust efficacy of “Second Generation” CAR modified T cells was demonstrated in clinical trials targeting the CD19 molecule in patients with chronic lymphoblastic leukemia (CLL) and acute lymphoblastic leukemia (ALL) (Davila et al., Oncoimmunol. 1(9):1577-1583 (2012)).

“Third generation” CARs provide multiple co-stimulation, for example, by comprising both CD28 and 4-1BB domains, and activation, for example, by comprising a CD3ζ activation domain.

“Fourth generation” CARs provide co-stimulation, for example, by CD28 or 4-1BB domains, and activation, for example, by a CD3ζ signaling domain in addition to a constitutive or inducible chemokine component.

“Fifth generation” CARs provide co-stimulation, for example, by CD28 or 4-1BB domains, and activation, for example, by a CD3ζ signaling domain, a constitutive or inducible chemokine component, and an intracellular domain of a cytokine receptor, for example, IL-2Rβ.

In various embodiments, the CAR can be included in a multivalent CAR system, for example, a DualCAR or “TandemCAR” system. Multivalent CAR systems include systems or cells comprising multiple CARs and systems or cells comprising bivalent/bispecific CARs targeting more than one antigen.

The present invention encompasses a number of CAR cells engineered for inhibiting or reducing the level of at least one cell of interest (e.g., alloresponsive cell, immune cell, T cell, B cell, natural killer cell, white blood cell, myeloid cell, plasma cell, etc.). In some embodiments, the CAR cell comprises at least one peptide and/or nucleic acid molecule of the present invention.

For example, in various embodiment, the CAR constructs can comprise a variety of CAR constructs known in the art, including but not limited to CAR disclosed in U.S. Pat. Nos. 8,906,682 B2, 8,911,993 B2, 8,916,381 B1, 8,975,071 B1, 9,101,584 B2, 9,102,760 B2, 9,102,761 B2, 9,328,156 B2, 9,464,140 B2, 9,481,728 B2, 9,499,629 B2, 9,518,123 B2, 9,540,445 B2, 9,573,988 B2, 10,040,846 B2, 10,117,896 B2, 10,174,095 B2, 10,221,245 B2, 10,308,717 B2, U.S. Patent Application No. 20170137783 A1, and/or U.S. Patent Application No. 20180258149 A1.

In one embodiment, the scFv portion of a CAR of the invention is encoded by a transgene whose sequence has been codon optimized for expression in a mammalian cell. In one embodiment, the entire CAR construct of the invention is encoded by a transgene whose entire sequence has been codon optimized for expression in a mammalian cell. Codon optimization refers to the discovery that the frequency of occurrence of synonymous codons (i.e., codons that code for the same amino acid) in coding DNA is biased in different species. Such codon degeneracy allows an identical polypeptide to be encoded by a variety of nucleotide sequences. A variety of codon optimization methods is known in the art, and include, e.g., methods disclosed in at least U.S. Pat. Nos. 5,786,464 and 6,114,148.

In some embodiments, the CAR cell comprises an antibody or a fragment thereof engineered for enhanced binding to at least one target of interest. The target-specific binding domain can be any domain that binds to a specific target including, but not limited to, target-specific binding domains derived from any one or more of monoclonal antibodies, polyclonal antibodies, recombinant antibodies, human antibodies, humanized antibodies, HLA I molecules, HLA II molecules, MHC I molecules, MHC II molecules, and fragments thereof, including, but not limited to, single-domain antibodies, such as a heavy chain variable domain (VH), a light chain variable domain (VL), and a variable domain (VHH) of camelid derived nanobody, and to alternative scaffolds known in the art that function as antigen binding domains, such as recombinant fibronectin domains, and the like. In some instances, it is beneficial for the target-specific binding domain to be derived from the same species in which the CAR will ultimately be used in. For example, for use in humans, it may be beneficial for the target-specific binding domain of the CAR to comprise human or humanized residues for the target-specific binding domain of an antibody or a fragment thereof. Thus, in one embodiment, the target-specific binding domain portion comprises a humanized antibody or a fragment thereof.

In some embodiments, anon-human antibody is humanized, where specific sequences or regions of the antibody are modified to increase similarity to an antibody naturally produced in a human or fragment thereof. In one embodiment, the target-specific binding domain portion is humanized. In one embodiment, the antigen binding domain portion is humanized.

A humanized antibody can be produced using a variety of techniques known in the art, including but not limited to, CDR-grafting (see, e.g., European Patent No. EP 239,400; International Publication No. WO 91/09967; and U.S. Pat. Nos. 5,225,539, 5,530,101, and 5,585,089, each of which is incorporated herein in its entirety by reference), veneering or resurfacing (see, e.g., European Patent Nos. EP 592,106 and EP 519,596; Padlan, 1991, Molecular Immunology, 28(4/5):489-498; Studnicka et al., 1994, Protein Engineering, 7(6):805-814; and Roguska et al., 1994, PNAS, 91:969-973, each of which is incorporated herein by its entirety by reference), chain shuffling (see, e.g., U.S. Pat. No. 5,565,332, which is incorporated herein in its entirety by reference), and techniques disclosed in, e.g., U.S. Patent Application Publication No. US2005/0042664, U.S. Patent Application Publication No. US2005/0048617, U.S. Pat. Nos. 6,407,213, 5,766,886, International Publication No. WO 9317105, Tan et al., J. Immunol., 169:1119-25 (2002), Caldas et al., Protein Eng., 13(5):353-60 (2000), Morea et al., Methods, 20(3):267-79 (2000), Baca et al., J. Biol. Chem., 272(16):10678-84 (1997), Roguska et al., Protein Eng., 9(10):895-904 (1996), Couto et al., Cancer Res., 55 (23 Supp):5973s-5977s (1995), Couto et al., Cancer Res., 55(8):1717-22 (1995), Sandhu J S, Gene, 150(2):409-10 (1994), and Pedersen et al., J. Mol. Biol., 235(3):959-73 (1994), each of which is incorporated herein in its entirety by reference. Often, framework residues in the framework regions will be substituted with the corresponding residue from the CDR donor antibody to alter, for example improve, antigen binding. These framework substitutions are identified by methods well-known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; and Riechmann et al., 1988, Nature, 332:323, which are incorporated herein by reference in their entireties.)

A humanized antibody or antibody fragment has one or more amino acid residues remaining in it from a source which is nonhuman. These nonhuman amino acid residues are often referred to as “import” residues, which are typically taken from an “import” variable domain. As provided herein, humanized antibodies or antibody fragments comprise one or more CDRs from nonhuman immunoglobulin molecules and framework regions wherein the amino acid residues comprising the framework are derived completely or mostly from human germline. Multiple techniques for humanization of antibodies or antibody fragments are well-known in the art and can essentially be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody, i.e., CDR-grafting (EP 239,400; PCT Publication No. WO 91/09967; and U.S. Pat. Nos. 4,816,567; 6,331,415; 5,225,539; 5,530,101; 5,585,089; 6,548,640, the contents of which are incorporated herein by reference herein in their entirety). In such humanized chimeric antibodies and antibody fragments, substantially less than an intact human variable domain has been substituted by the corresponding sequence from a nonhuman species. In practice, humanized antibodies are typically human antibodies in which some CDR residues and possibly some framework (FR) residues are substituted by residues from analogous sites in rodent antibodies. Humanization of antibodies and antibody fragments can also be achieved by veneering or resurfacing (EP 592,106; EP 519,596; Padlan, 1991, Molecular Immunology, 28(4/5):489-498; Studnicka et al, Protein Engineering, 7(6):805-814 (1994); and Roguska et al., PNAS, 91:969-973 (1994)) or chain shuffling (U.S. Pat. No. 5,565,332), the contents of which are incorporated herein by reference herein in their entirety.

The choice of human variable domains, both light and heavy, to be used in making the humanized antibodies is to reduce antigenicity. According to the so-called “best-fit” method, the sequence of the variable domain of a rodent antibody is screened against the entire library of known human variable-domain sequences. The human sequence which is closest to that of the rodent is then accepted as the human framework (FR) for the humanized antibody (Sims et al., J. Immunol., 151:2296 (1993); Chothia et al., J. Mol. Biol., 196:901 (1987), the contents of which are incorporated herein by reference herein in their entirety). Another method uses a particular framework derived from the consensus sequence of all human antibodies of a particular subgroup of light or heavy chains. The same framework may be used for several different humanized antibodies (see, e.g., Nicholson et al. Mol. Immun. 34 (16-17): 1157-1165 (1997); Carter et al., Proc. Natl. Acad. Sci. USA, 89:4285 (1992); Presta et al., J. Immunol., 151:2623 (1993), the contents of which are incorporated herein by reference herein in their entirety).

In various embodiments, the portion of the CAR composition that comprises an antibody or a fragment thereof is humanized with retention of high affinity for the target antigen, target cell bearing antigen, target alloresponsive cell bearing antigen, target TCR, target T cell bearing TCR, target alloresponsive T cell bearing TCR, target BCR, target B cell bearing BCR, target alloresponsive B cell bearing BCR, target immune cell, target T cell, target B cell, target natural killer cell, target white blood cell, target myeloid cell, target plasma cell, or any combination thereof, and other favorable biological properties.

In one embodiment, humanized antibodies and antibody fragments are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind the target antigen. In this way, FR residues can be selected and combined from the recipient and import sequences so that the desired antibody or antibody fragment characteristic, such as increased affinity for the target antigen, is achieved. In general, the CDR residues are directly and most substantially involved in influencing antigen binding.

A humanized antibody or antibody fragment retains a similar antigenic specificity as the original antibody. However, the methods disclosed herein may further improve the affinity and/or specificity of binding of the antibody for human cell bearing antigen. In one embodiment, the target-specific binding domain is characterized by particular functional features or properties of an antibody or antibody fragment.

In one aspect, the invention relates to an antigen binding domain comprising an antibody or antibody fragment, wherein the antibody binding domain specifically binds to a target of interest or fragment thereof. In one embodiment, the scFv is contiguous with and in the same reading frame as a leader sequence.

In one embodiment, the antibody fragment provided herein is a single chain variable fragment (scFv). In various embodiments, the antibodies of the invention may exist in a variety of other forms including, for example, Fv, Fab, and (Fab′) 2, as well as bi-functional (i.e. bi-specific) hybrid antibodies (e.g., Lanzavecchia et al., Eur. J. Immunol. 17, 105 (1987)).

ScFvs can be prepared according to method known in the art (see, for example, Bird et al., (1988) Science 242:423-426 and Huston et al., (1988) Proc. Natl. Acad. Sci. USA 85:5879-5883). ScFv molecules can be produced by linking VH and VL regions together using flexible polypeptide linkers. The scFv molecules comprise flexible polypeptide linker (e.g., a Ser-Gly linker) with an optimized length and/or amino acid composition. The flexible polypeptide linker length can greatly affect how the variable regions of an scFv fold and interact. In fact, if a short polypeptide linker is employed (e.g., between 5-10 amino acids, intrachain folding is prevented. Interchain folding is also required to bring the two variable regions together to form a functional epitope binding site. For examples of linker orientation and size see, e.g., Hollinger et al. 1993 Proc Natl Acad. Sci. U.S.A. 90:6444-6448, U.S. Patent Application Publication Nos. 2005/0100543, 2005/0175606, 2007/0014794, and PCT publication Nos. WO2006/020258 and WO2007/024715, is incorporated herein by reference.

The scFv can comprise a polypeptide linker sequence of at least 1, 2, 3, 4, 5, 6, 7, 8, 9, 10, 11, 12, 13, 14, 15, 16, 17, 18, 19, 20, 25, 30, 35, 40, 45, 50, or more amino acid residues between its VL and VH regions. The flexible polypeptide linker sequence may comprise any naturally occurring amino acid. In some embodiments, the flexible polypeptide linker sequence comprises amino acids glycine and serine. In another embodiment, the flexible polypeptide linker sequence comprises sets of glycine and serine repeats such as (Gly4Ser)n, where n is a positive integer equal to or greater than 1. In one embodiment, the flexible polypeptide linkers include, but are not limited to, (Gly4Ser)4 or (Gly4Ser)3. Variation in the flexible polypeptide linker length may retain or enhance activity, giving rise to superior efficacy in activity studies.

### Stability and Mutations

The stability of scFv molecules (e.g., soluble scFv) can be evaluated in reference to the biophysical properties (e.g., thermal stability) of a conventional control scFv molecule or a full length antibody. In one embodiment, the humanized soluble scFv has a thermal stability that is greater than about 0.1, about 0.25, about 0.5, about 0.75, about 1, about 1.25, about 1.5, about 1.75, about 2, about 2.5, about 3, about 3.5, about 4, about 4.5, about 5, about 5.5, about 6, about 6.5, about 7, about 7.5, about 8, about 8.5, about 9, about 9.5, about 10 degrees, about 11 degrees, about 12 degrees, about 13 degrees, about 14 degrees, or about 15 degrees Celsius than a control binding molecule (e.g. a conventional scFv molecule) in the described assays.

The improved thermal stability of the scFv is subsequently conferred to the entire CAR construct, leading to improved therapeutic properties of the CAR construct. The thermal stability of the scFv can be improved by at least about 2° C. or 3° C. as compared to a conventional antibody. In one embodiment, the scFv has a 1° C. improved thermal stability as compared to a conventional antibody. In another embodiment, the scFv has a 2° C. improved thermal stability as compared to a conventional antibody. In another embodiment, the scFv has a 4, 5, 6, 7, 8, 9, 10, 11, 12, 13, 14, 15° C. improved thermal stability as compared to a conventional antibody. Comparisons can be made, for example, between the scFv molecules disclosed herein and scFv molecules or Fab fragments of an antibody from which the scFv VH and VL were derived. Thermal stability can be measured using methods known in the art. For example, in one embodiment, Tm can be measured. Methods for measuring Tm and other methods of determining protein stability are described in more detail below.

Mutations in scFv (arising through humanization or direct mutagenesis of the soluble scFv) alter the stability of the scFv and improve the overall stability of the scFv and the CAR construct. Stability of the humanized scFv is compared against the murine scFv using measurements such as Tm, temperature denaturation and temperature aggregation. The residues introduced by humanization thereby have improved the Tm of the scFv by more than 10° C.

In one embodiment, the scFv comprises at least one mutation arising from the humanization process such that the mutated scFv confers improved stability to the CAR construct. In various embodiments, the scFv comprises at least 1, 2, 3, 4, 5, 6, 7, 8, 9, 10 mutations arising from the humanization process such that the mutated scFv confers improved stability to the CAR construct. The binding capacity of the mutant scFvs can be determined using any of the assays described herein.

In some embodiments, the engineered TCR comprises one or more TCR chains (e.g., TCR alpha chain, TCR beta chain, TCR delta chain, and TCR gamma chain) that, either alone or together, specifically bind to a target of interest, such as a target of interest on a cancer cell. In one embodiment, the engineered TCR comprises a TCR alpha chain and a TCR beta chain, where the engineered TCR specifically binds to target of interest.

### Methods of Evaluating Protein Stability

To assess the stability of scFv constructs, the stability of the minimal domain of a multidomain protein, i.e., a scFv of a CAR construct, is predicted using the methods and those described below. Such methods allow for the determination of multiple thermal unfolding transitions where the least stable domain either unfolds first or limits the overall stability threshold of a multidomain unit that unfolds cooperatively (i.e., a multidomain protein which exhibits a single unfolding transition). The least stable domain can be identified in a number of additional ways. Mutagenesis can be performed to probe which domain limits the overall stability. Additionally, protease resistance of a multidomain protein can be performed under conditions where the least stable domain is known to be intrinsically unfolded via DSC or other spectroscopic methods (Fontana, et al., 1997, Fold. Des., 2: R17-26; Dimasi et al., 2009, J. Mol. Biol. 393: 672-692). Once the least stable domain is identified, the sequence encoding this domain (or a portion thereof) may be employed as a test sequence in the methods.

### a) Thermal Stability

The thermal stability of the compositions may be analyzed using a number of non-limiting biophysical or biochemical techniques known in the art. In certain embodiments, thermal stability is evaluated by analytical spectroscopy.

An exemplary analytical spectroscopy method is Differential Scanning Calorimetry (DSC). DSC employs a calorimeter which is sensitive to the heat absorbances that accompany the unfolding of most proteins or protein domains (e.g., Sanchez-Ruiz et al., 1988, Biochemistry, 27: 1648-1652). To determine the thermal stability of a protein, a sample of the protein is inserted into the calorimeter and the temperature is raised until the Fab or scFv unfolds. The temperature at which the protein unfolds is indicative of overall protein stability.

Another exemplary analytical spectroscopy method is Circular Dichroism (CD) spectroscopy. CD spectrometry measures the optical activity of a composition as a function of increasing temperature. Circular dichroism (CD) spectroscopy measures differences in the absorption of left-handed polarized light versus right-handed polarized light which arise due to structural asymmetry. A disordered or unfolded structure results in a CD spectrum very different from that of an ordered or folded structure. The CD spectrum reflects the sensitivity of the proteins to the denaturing effects of increasing temperature and is therefore indicative of a protein's thermal stability (van Mierlo and Steemsma, J. Biotechnol., 2000, 79:281-298).

Another exemplary analytical spectroscopy method for measuring thermal stability is Fluorescence Emission Spectroscopy (van Mierlo and Steemsma, supra). Yet another exemplary analytical spectroscopy method for measuring thermal stability is Nuclear Magnetic Resonance (NMR) spectroscopy (e.g., van Mierlo and Steemsma, supra).

The thermal stability of a composition can be measured biochemically. An exemplary biochemical method for assessing thermal stability is a thermal challenge assay. In a “thermal challenge assay”, a composition is subjected to a range of elevated temperatures for a set period of time. For example, in one embodiment, test scFv molecules or molecules comprising scFv molecules are subject to a range of increasing temperatures, e.g., for 1-1.5 hours. The activity of the protein is then assayed by a relevant biochemical assay. For example, if the protein is a binding protein (e.g. an scFv or scFv-containing polypeptide) the binding activity of the binding protein may be determined by a functional or quantitative ELISA.

Such an assay may be done in a high-throughput format and those disclosed in the Examples using E. coli and high throughput screening. A library of scFv variants may be created using methods known in the art. scFv expression may be induced an scFvs may be subjected to thermal challenge. The challenged test samples may be assayed for binding and those scFvs which are stable may be scaled up and further characterized.

Thermal stability is evaluated by measuring the melting temperature (Tm) of a composition using any of the above techniques (e.g. analytical spectroscopy techniques). The melting temperature is the temperature at the midpoint of a thermal transition curve wherein 50% of molecules of a composition are in a folded state (e.g., Dimasi et al., 2009, J. Mol Biol. 393: 672-692). In one embodiment, Tm values for a scFv are about 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., 80° C., 81° C., 82° C., 83° C., 84° C., 85° C., 86° C., 87° C., 88° C., 89° C., 90° C., 91° C., 92° C., 93° C., 94° C., 95° C., 96° C., 97° C., 98° C., 99° C., 100° C. In one embodiment, Tm values for an IgG is about 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., 80° C., 81° C., 82° C., 83° C., 84° C., 85° C., 86° C., 87° C., 88° C., 89° C., 90° C., 91° C., 92° C., 93° C., 94° C., 95° C., 96° C., 97° C., 98° C., 99° C., 100° C. In one embodiment, Tm values for an multivalent antibody is about 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., 80° C., 81° C., 82° C., 83° C., 84° C., 85° C., 86° C., 87° C., 88° C., 89° C., 90° C., 91° C., 92° C., 93° C., 94° C., 95° C., 96° C., 97° C., 98° C., 99° C., 100° C.

Thermal stability is also evaluated by measuring the specific heat or heat capacity (Cp) of a composition using an analytical calorimetric technique (e.g. DSC). The specific heat of a composition is the energy (e.g. in kcal/mol) is required to rise by 1° C., the temperature of 1 mol of water. As large Cp is a hallmark of a denatured or inactive protein composition. The change in heat capacity (ΔCp) of a composition is measured by determining the specific heat of a composition before and after its thermal transition. Thermal stability may also be evaluated by measuring or determining other parameters of thermodynamic stability including Gibbs free energy of unfolding (ΔG), enthalpy of unfolding (ΔH), or entropy of unfolding (ΔS). One or more of the above biochemical assays (e.g. a thermal challenge assay) are used to determine the temperature (i.e. the TC value) at which 50% of the composition retains its activity (e.g. binding activity).

In addition, mutations to the scFv alter the thermal stability of the scFv compared with the unmutated scFv. When the humanized scFv is incorporated into a CAR construct, the humanized scFv confers thermal stability to the overall anti-CAR construct. In one embodiment, the scFv comprises a single mutation that confers thermal stability to the scFv. In another embodiment, the scFv comprises multiple mutations that confer thermal stability to the scFv. In one embodiment, the multiple mutations in the scFv have an additive effect on thermal stability of the scFv.

### b) % Aggregation

The stability of a composition can be determined by measuring its propensity to aggregate. Aggregation can be measured by a number of non-limiting biochemical or biophysical techniques. For example, the aggregation of a composition may be evaluated using chromatography, e.g. Size-Exclusion Chromatography (SEC). SEC separates molecules on the basis of size. A column is filled with semi-solid beads of a polymeric gel that will admit ions and small molecules into their interior but not large ones. When a protein composition is applied to the top of the column, the compact folded proteins (i.e. non-aggregated proteins) are distributed through a larger volume of solvent than is available to the large protein aggregates. Consequently, the large aggregates move more rapidly through the column, and in this way the mixture can be separated or fractionated into its components. Each fraction can be separately quantified (e.g., by light scattering) as it elutes from the gel. Accordingly, the % aggregation of a composition can be determined by comparing the concentration of a fraction with the total concentration of protein applied to the gel. Stable compositions elute from the column as essentially a single fraction and appear as essentially a single peak in the elution profile or chromatogram.

### c) Binding Affinity

The stability of a composition can be assessed by determining its target binding affinity. A wide variety of methods for determining binding affinity are known in the art. An exemplary method for determining binding affinity employs surface plasmon resonance. Surface plasmon resonance is an optical phenomenon that allows for the analysis of real-time biospecific interactions by detection of alterations in protein concentrations within a biosensor matrix, for example using the BIAcore system (Pharmacia Biosensor AB, Uppsala, Sweden and Piscataway, N.J.). For further descriptions, see Jonsson, U., et al. (1993) Ann. Biol. Clin. 51:19-26; Jonsson, U., i (1991) Biotechniques 11:620-627; Johnsson, B., et al. (1995) J. Mol. Recognit. 8:125-131; and Johnnson, B., et al. (1991) Anal. Biochem. 198:268-277.

In various embodiments, the portion of a CAR composition of the invention comprising an antibody or antibody fragment further comprises heavy and light chain variable regions comprising amino acid sequences that are homologous to the amino acid sequences of the antibodies described herein, and wherein the antibodies retain the desired functional properties of the antibodies of the invention. In one embodiment, the CAR composition comprises an antibody fragment. In one embodiment, the antibody fragment comprises an scFv.

In various embodiments, the portion comprising an antibody or antibody fragment of the CAR composition is engineered by modifying one or more amino acids within one or both variable regions (i.e., VH and/or VL). For example, the antibody or antibody fragment of the CAR composition is engineered by modifying one or more amino acids within one or more CDR regions and/or within one or more framework regions.

In some embodiments, the CAR cell comprises an extracellular receptor, a transmembrane domain, and an intracellular signaling domain. In one embodiment, the extracellular receptor is connected to the intracellular signaling domain. In one embodiment, the extracellular receptor is connected to the intracellular signaling domain through the transmembrane domain to the intracellular signaling domain.

In some embodiments, the CAR cells comprises a recombinant DNA construct comprising sequences encoding the CAR disclosed therein.

The nucleic acid sequences coding for the desired molecules can be obtained using recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned.

The nucleic acid sequences coding for the desired molecules can be obtained using recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned.

In some embodiments, in order to assess the expression of a peptide, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2012, Molecular Cloning: A Laboratory Manual, Cold Spring Harbor Laboratory, New York). An exemplary method for the introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, MO; dicetyl phosphate (“DCP”) can be obtained from K & K Laboratories (Plainview, NY); cholesterol (“Choi”) can be obtained from Calbiochem-Behring; dimyristyl phosphatidylglycerol (“DMPG”) and other lipids may be obtained from Avanti Polar Lipids, Inc. (Birmingham, AL). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

RNA can be introduced into target cells using any of a number of different methods, for instance, commercially available methods which include, but are not limited to, electroporation (Amaxa Nucleofector-II (Amaxa Biosystems, Cologne, Germany)), (ECM 830 (BTX) (Harvard Instruments, Boston, Mass.) or the Gene Pulser II (BioRad, Denver, Colo.), Multiporator (Eppendort, Hamburg Germany), cationic liposome mediated transfection using lipofection, polymer encapsulation, peptide mediated transfection, or biolistic particle delivery systems such as “gene guns” (see, for example, Nishikawa, et al. Hum Gene Ther., 12(8):861-70 (2001).

### Methods of Use

The present invention also relates, in part, to a method of reducing clearance or increasing persistence of at least one cell of interest (e.g., donor cell, such as T cell, CAR cell, CAR T cell, engineered TCR-expressing T cell, etc.). In various aspects, the present invention provides a method of preventing, reducing, or eliminating an alloresponse; a method of decreasing, reducing, eliminating, or depleting a level of alloresponsive cells, and a method of inducing allogeneic tolerance or unresponsiveness of allogeneic cells. In various aspects, the present invention also provides a method of improving the effectiveness of a CAR therapy or engineered TCR cell therapy.

In various aspects, the present invention also provides a method of preventing or treating various disease or disorder. In some embodiments, the method comprises an administration of a therapeutically effective of any of the nucleic acid molecules, compositions, and/or cells described herein. For example, in one embodiment, the method comprises an administration of a therapeutically effective of at least one genetically engineered TKO cell that expresses at least one peptide (e.g., SCT) of the invention. In one embodiment, the method comprises an administration of a therapeutically effective of at least one CAR T cell that expresses at least one peptide (e.g., SCT) of the invention. In one embodiment, the method comprises an administration of a therapeutically effective of at least one engineered TCR-expressing T cell that expresses at least one peptide (e.g., SCT) of the invention.

In some embodiments, the disease or disorder comprises a disease or disorder associated with expression of alloresponsive cells, disease or disorder associated with at least one HLA receptor, disease or disorder associated with at least one HLA-containing receptor, disease or disorder associated with at least one MHC receptor, disease or disorder associated with at least one MHC-containing receptor, GvHD, autoimmune disease or disorder, disease or disorder associated with organ transplantation, disease or disorder associated with tissue transplantation, disease or disorder associated with cell transplantation, disease or disorder associated with allotransplantation, disease or disorder associated with intestinal transplantation, disease or disorder associated with reconstructive transplantation, cancer, disease or disorder associated with cancer, or any combination thereof.

In various aspects, the present invention provides a method of preventing or treating a cancer, disease or disorder associated with cancer, or a combination thereof in a subject in need thereof, the method comprising administering an effective amount of at least one peptide, nucleic acid molecule, cell, or composition of the present invention to the subject. For example, in one embodiment, the method is an engineered TCR cell therapy.

In some aspects, the present invention provides methods of administering an effective amount of at least one peptide, nucleic acid molecule, cell, or composition of the present invention to the subject. In some embodiments, the subject has a cancer, disease or disorder associated with cancer, or a combination thereof.

The following are non-limiting examples of cancers that can be treated by the disclosed methods and compositions: acute lymphoblastic; acute myeloid leukemia; adrenocortical carcinoma; adrenocortical carcinoma, childhood; appendix cancer; basal cell carcinoma; bile duct cancer, extrahepatic; bladder cancer; bone cancer; osteosarcoma and malignant fibrous histiocytoma; brain stem glioma, childhood; brain tumor, adult; brain tumor, brain stem glioma, childhood; brain tumor, central nervous system atypical teratoid/rhabdoid tumor, childhood; central nervous system embryonal tumors; cerebellar astrocytoma; cerebral astrocytoma/malignant glioma; craniopharyngioma; ependymoblastoma; ependymoma; medulloblastoma; medulloepithelioma; pineal parenchymal tumors of intermediate differentiation; supratentorial primitive neuroectodermal tumors and pineoblastoma; visual pathway and hypothalamic glioma; brain and spinal cord tumors; breast cancer; bronchial tumors; Burkitt lymphoma; carcinoid tumor; carcinoid tumor, gastrointestinal; central nervous system atypical teratoid/rhabdoid tumor; central nervous system embryonal tumors; central nervous system lymphoma; cerebellar astrocytoma cerebral astrocytoma/malignant glioma, childhood; cervical cancer; chordoma, childhood; chronic lymphocytic leukemia; chronic myelogenous leukemia; chronic myeloproliferative disorders; colon cancer; colorectal cancer; craniopharyngioma; cutaneous T-cell lymphoma; esophageal cancer; Ewing family of tumors; extragonadal germ cell tumor; extrahepatic bile duct cancer; eye cancer, intraocular melanoma; eye cancer, retinoblastoma; gallbladder cancer; gastric (stomach) cancer; gastrointestinal carcinoid tumor; gastrointestinal stromal tumor (gist); germ cell tumor, extracranial; germ cell tumor, extragonadal; germ cell tumor, ovarian; gestational trophoblastic tumor; glioma; glioma, childhood brain stem; glioma, childhood cerebral astrocytoma; glioma, childhood visual pathway and hypothalamic; hairy cell leukemia; head and neck cancer; hepatocellular (liver) cancer; histiocytosis, langerhans cell; Hodgkin lymphoma; hypopharyngeal cancer; hypothalamic and visual pathway glioma; intraocular melanoma; islet cell tumors; kidney (renal cell) cancer; Langerhans cell histiocytosis; laryngeal cancer; leukemia, acute lymphoblastic; leukemia, acute myeloid; leukemia, chronic lymphocytic; leukemia, chronic myelogenous; leukemia, hairy cell; lip and oral cavity cancer; liver cancer; lung cancer, non-small cell; lung cancer, small cell; lymphoma, aids-related; lymphoma, burkitt; lymphoma, cutaneous T-cell; lymphoma, non-Hodgkin lymphoma; lymphoma, primary central nervous system; macroglobulinemia, Waldenstrom; malignant fibrous histiocytoma of bone and osteosarcoma; medulloblastoma; melanoma; melanoma, intraocular (eye); Merkel cell carcinoma; mesothelioma; metastatic squamous neck cancer with occult primary; mouth cancer; multiple endocrine neoplasia syndrome, (childhood); multiple myeloma/plasma cell neoplasm; mycosis; fungoides; myelodysplastic syndromes; myelodysplastic/myeloproliferative diseases; myelogenous leukemia, chronic; myeloid leukemia, adult acute; myeloid leukemia, childhood acute; myeloma, multiple; myeloproliferative disorders, chronic; nasal cavity and paranasal sinus cancer; nasopharyngeal cancer; neuroblastoma; non-small cell lung cancer; oral cancer; oral cavity cancer; oropharyngeal cancer; osteosarcoma and malignant fibrous histiocytoma of bone; ovarian cancer; ovarian epithelial cancer; ovarian germ cell tumor; ovarian low malignant potential tumor; pancreatic cancer; pancreatic cancer, islet cell tumors; papillomatosis; parathyroid cancer; penile cancer; pharyngeal cancer; pheochromocytoma; pineal parenchymal tumors of intermediate differentiation; pineoblastoma and supratentorial primitive neuroectodermal tumors; pituitary tumor; plasma celt neoplasm/multiple myeloma; pleuropulmonary blastoma; primary central nervous system lymphoma; prostate cancer; rectal cancer; renal cell (kidney) cancer; renal pelvis and ureter, transitional cell cancer; respiratory tract carcinoma involving the nut gene on chromosome 15; retinoblastoma; rhabdomyosarcoma; salivary gland cancer; sarcoma, ewing family of tumors; sarcoma, Kaposi; sarcoma, soft tissue; sarcoma, uterine; sezary syndrome; skin cancer (nonmelanoma); skin cancer (melanoma); skin carcinoma, Merkel cell; small cell lung cancer; small intestine cancer; soft tissue sarcoma; squamous cell carcinoma, squamous neck cancer with occult primary, metastatic; stomach (gastric) cancer; supratentorial primitive neuroectodermal tumors; T-cell lymphoma, cutaneous; testicular cancer; throat cancer; thymoma and thymic carcinoma; thyroid cancer; transitional cell cancer of the renal pelvis and ureter; trophoblastic tumor, gestational; urethral cancer; uterine cancer, endometrial; uterine sarcoma; vaginal cancer; vulvar cancer; Waldenstrom macroglobulinemia; and Wilms tumor.

In some aspects, the present invention also provides methods comprising administering an effective amount of any peptide, nucleic acid molecule, cell, or composition disclosed herein to the subject, wherein the subject has at least one donor cell. In some aspects, the present invention also provides methods comprising administering an effective amount of any peptide, nucleic acid molecule, cell, or composition disclosed herein to the subject, wherein the subject is in a need of reducing clearance or increasing persistence of at least one cell of interest (e.g., donor cell, such as T cell, CAR cell, CAR T cell, engineered TCR-expressing T cell, etc.).

In one embodiment, the invention provides methods for preventing or treating a disease or disorder associated with alloresponsive cell expression. In various embodiments, the disease or disorder associated with expression of alloresponsive cells is an allograft rejection, an immune rejection, a chronic allogeneic rejection, an engraftment rejection, a transplant rejection, an inflammation, an inflammation caused by ischemia/reperfusion, an infection, an immune response to an allograft, or any combination thereof. In various embodiments, the disease or disorder associated with expression of alloresponsive cells are caused by an organ transplantation, cell transplantation, cell transplantation. allotransplantation, intestinal transplantation, reconstructive transplantation, autoimmune disease or disorder, disease or disorder associated with at least one HLA receptor, disease or disorder associated with at least one HLA-containing receptor, disease or disorder associated with at least one MHC receptor, disease or disorder associated with at least one MHC-containing receptor, or any combination thereof.

In various embodiments, the alloresponsive cells are cells bearing antigens, TCRs, BCRs, alloresponsive immune cells, allo-specific immune cells, allogeneic immune cells, autologous immune cell, immune cells bearing alloreactive antigen, TCR, and/or BCR, alloresponsive T cells, allo-specific T cells, allogeneic T cells, autologous T cell, T cells bearing alloreactive TCR, alloresponsive B cells, allo-specific B cells, allogeneic B cells, autologous B cell, B cells bearing alloreactive BCR, or any combination thereof. For example, in various embodiments, the alloresponsive T cells are T cells bearing TCRs, alloresponsive T cells, allo-specific T cells, allogeneic T cells, autologous T cell, T cells bearing alloreactive TCR, or any combination thereof. In various embodiments, the alloresponsive B cells are B cells bearing BCRs, alloresponsive B cells, allo-specific B cells, allogeneic B cells, autologous B cell, B cells bearing alloreactive BCR, or any combination thereof.

In one embodiment, the method comprises administering at least one cell genetically modified to express at least one peptide (e.g., SCT) of the invention, wherein the peptide specifically inhibits NK cells.

Furthermore, the present invention provides nucleic acid molecules and compositions and cells comprising thereof as well as their use in medicaments or methods for preventing, reducing, and/or eliminating an alloresponse, methods for the eliminating specific cell population, methods for depleting a level of alloresponsive cells (e.g., T cell), or methods of inducting allogeneic tolerance, and/or immune unresponsiveness to an allograft. For example, the nucleic acid molecule of the invention is useful for treating subjects that have undergone treatment for a disease or disorder associated with expression of alloresponsive T cells, wherein the subject that has undergone treatment for expression of alloresponsive T cells exhibits a disease associated with expression of alloresponsive cells.

In one embodiment, the invention pertains to a vector comprising the nucleic acid molecules described herein operably linked to promoter for expression in mammalian cells (e.g., mammalian T cells, mammalian B cells, etc.). For example, in one embodiment, the invention provides a recombinant cell expressing at least one peptide (e.g., SCT) of the invention for use in preventing and/or treating disease or disorder associated with alloresponsive cells. In one embodiment, the cell of the invention is capable of contacting an alloresponsive cell with at least one peptide (e.g., SCT) of the invention expressed on its surface such that at least one NK cell is inhibited and alloresponse is inhibited.

For example, in one embodiment, the invention provides a recombinant T cell expressing at least one peptide (e.g., SCT) of the invention for use in preventing and/or treating disease or disorder associated with alloresponsive T cells.

In one embodiment, the cell of the invention is capable of contacting an alloresponsive cell with at least one peptide (e.g., SCT) of the invention expressed on its surface such that at least one NK cell is inhibited and alloresponse is inhibited. In one embodiment, the cell of the invention is capable of contacting an alloresponsive cell with at least one peptide (e.g., SCT) of the invention expressed on its surface such that at least one NK cell is inhibited and allogenic tolerance is induced.

In one embodiment, the present invention includes cellular therapy where cells are modified to comprise or express at least one peptide (e.g., SCT) of the invention, and the cell is infused to a recipient in need thereof.

In one embodiment, the present invention includes cellular therapy where the method comprises administering to a subject a composition comprising a cell, such as an antigen presenting cell, that comprises or expresses at least one peptide (e.g., SCT) of the invention. For example, in one embodiment, the method comprises administering a composition comprising an antigen presenting cell that is loaded with at least one peptide (e.g., SCT) of the invention and expresses the at least one peptide (e.g., SCT) on the surface.

In one embodiment, the present invention includes cellular therapy where the method comprises administering to a subject a composition comprising a cell that is activated or stimulated by an antigen presenting cell that that comprises or expresses at least one peptide (e.g., SCT) of the invention. For example, in one embodiment, the method comprises contacting a cell, such as a naïve T cell, to an antigen presenting cell that is loaded with at least one peptide (e.g., SCT) of the invention and expresses the at least one peptide (e.g., SCT) on the surface; thereby activating the cell. The method comprises administering a composition comprising the activated cell to a subject. For example, in one embodiment, the method of the invention comprises the following steps: (1) providing a population of naïve T cells; (2) providing a population of dendritic cells; (3) loading or pulsing the dendritic cells with one or more peptides (e.g., SCT) of the invention; (4) co-culturing the naïve T cells and loaded dendritic cells; and (5) isolating the stimulated T cells. In one embodiment, the method further comprises step (6) administering the stimulated T cells to a subject in need thereof.

In certain embodiments, the modified cell (e.g., an antigen presenting cell presenting at least one peptide (e.g., SCT) of the invention) is able to prevent or reduce an immune response in vivo. For example, in certain embodiments, the modified cell is able to prevent or reduce a rejection of transplant in the recipient.

In one embodiment, the modified T cells of the invention can undergo robust in vivo T cell expansion and can persist for an extended amount of time. For example, modified T cells of the invention can undergo robust in vivo T cell expansion and persist at high levels for an extended amount of time in blood and bone marrow and form specific memory T cells.

In some embodiments, the invention includes a type of cellular therapy where cells are genetically engineered to express an engineered TCR or chimeric antigen receptor (CAR) and the engineered TCR cell or CAR cell (e.g., CAR immune cell, CAR T cell, CAR B cell, etc.) is infused to a recipient in need thereof. The infused cell is able to kill or lyse alloresponsive cells in the recipient. Unlike antibody therapies, engineered TCR cells and CAR-modified cells are able to replicate in vivo resulting in long-term persistence that can lead to sustained alloresponse control. In various embodiments, the cells administered to the patient, or their progeny, persist in the patient for at least four months, five months, six months, seven months, eight months, nine months, ten months, eleven months, twelve months, thirteen months, fourteen month, fifteen months, sixteen months, seventeen months, eighteen months, nineteen months, twenty months, twenty-one months, twenty-two months, twenty-three months, two years, three years, four years, or five years after administration of the cell to the patient.

In one embodiment, the alloresponse suppression elicited by the engineered TCR cells or CAR-modified cells may be an active or a passive immune response, or alternatively may be due to a direct vs indirect immune response. In one embodiment, the engineered TCR cells or CAR transduced cells exhibit specific proinflammatory cytokine secretion and potent cytolytic activity in response to human alloresponsive cells expressing the antigen, TCR, and/or BCR.

With respect to ex vivo immunization, at least one of the following occurs in vitro prior to administering the cell into a mammal: i) expansion of the cells, ii) introducing a nucleic acid encoding a CAR to the cells or iii) cryopreservation of the cells.

Ex vivo procedures are well known in the art and are discussed more fully below. Briefly, cells are isolated from a mammal (e.g., a human) and genetically modified (i.e., transduced or transfected in vitro) with a vector expressing a CAR disclosed herein. The CAR-modified cell can be administered to a mammalian recipient to provide a therapeutic benefit. The mammalian recipient may be a human and the CAR-modified cell can be autologous with respect to the recipient. Alternatively, the cells can be allogeneic, syngeneic or xenogeneic with respect to the recipient.

The procedure for ex vivo expansion of hematopoietic stem and progenitor cells is described in U.S. Pat. No. 5,199,942, incorporated herein by reference, can be applied to the cells of the present invention. Other suitable methods are known in the art, therefore the present invention is not limited to any particular method of ex vivo expansion of the cells. Briefly, ex vivo culture and expansion of cells comprises: (1) collecting CD34+ hematopoietic stem and progenitor cells from a mammal from peripheral blood harvest or bone marrow explants; and (2) expanding such cells ex vivo. In addition to the cellular growth factors described in U.S. Pat. No. 5,199,942, other factors such as flt3 L, IL-1, IL-3 and c-kit ligand, can be used for culturing and expansion of the cells.

In some embodiments, the CAR-modified cells and engineered TCR cells of the invention are used in the treatment of diseases, disorders and disorders associated with expression of alloresponsive cells. In various embodiments, the cells of the invention are used in the treatment of patients at risk for developing diseases, disorders and disorders associated with expression of alloresponsive cells. Thus, the present invention provides methods for the treatment or prevention of diseases, disorders and disorders associated with expression of alloresponsive cells comprising administering to a subject in need thereof, a therapeutically effective amount of the genetically engineered cells of the invention.

The present invention also provides methods for inhibiting the proliferation or reducing an alloresponsive cell population, the methods comprising contacting a population of alloresponsive cells with at least one genetically engineered cell of the invention that binds to the alloresponsive cell. In one embodiment, the present invention provides methods for inhibiting the proliferation or reducing the population of alloresponsive cells bearing antigen, TCR, and/or BCR, the methods comprising contacting the alloresponsive cell population with at least one genetically engineered cell of the invention that binds to the antigen, TCR domain, and/or BCR domain of the alloresponsive cells. In various embodiments, the cell of the invention reduces the quantity, number, amount or percentage of cells and/or alloresponsive cells by at least 1%, at least 10%, at least 25%, at least 30%, at least 40%, at least 50%, at least 65%, at least 75%, at least 85%, at least 95%, or at least 99% in a subject with or animal model for disease or disorder associated with expression of alloresponsive cells relative to a negative control. In one embodiment, the subject is a human.

The present invention also provides methods for preventing, treating and/or managing a disease associated with expression of alloresponsive cells, the methods comprising administering to a subject in need at least one genetically engineered cell of the invention that binds to the alloresponsive cells. The present invention also provides methods for preventing, treating and/or managing a disease associated with expression of cells bearing antigen, TCR, and/or BCR, the methods comprising administering to a subject in need at least one genetically engineered cell of the invention that binds to the antigen-, TCR-, and/or BCR-expressing cell. In one embodiment, the subject is a human. Non-limiting examples of disorders associated with expression of alloresponsive cells include autoimmune disease or disorder (e.g., lupus), inflammatory disease or disorder (e.g., allergies and asthma), disease or disorder caused by transplantation, an allograft rejection, an immune rejection, a chronic allogeneic rejection, an engraftment rejection, a transplant rejection, or any combination thereof, an inflammation, an inflammation caused by ischemia/reperfusion, an infection, an immune response to an allograft, or any combination thereof.

The present invention provides methods for preventing relapse of disease or disorder associated with expression of alloresponsive cells, the methods comprising administering to a subject in need thereof at least one genetically engineered cell of the invention that binds to the alloresponsive cells. In one embodiment, the methods comprise administering to the subject in need thereof multiple effective amounts of at least one genetically engineered cell of the invention that binds to the alloresponsive cells. In one embodiment, the methods comprise administering to the subject in need thereof an effective amount of at least one genetically engineered cell of the invention that binds to the alloresponsive cells in combination with an effective amount of another therapy.

The compositions and genetically engineered cells of the present invention may be administered either alone, or as a pharmaceutical composition in combination with diluents and/or with other components, such as IL-2, or other cytokines or cell populations.

In a further embodiment, the compositions of the present invention are administered to a patient in conjunction with (e.g., before, simultaneously or following) transplantation (e.g., bone marrow transplantation, organ transplantation, etc.). In certain embodiments, following the transplant, subjects receive an infusion of the expanded immune cells of the present invention. In an additional embodiment, expanded cells are administered before or following surgery.

Subjects to which administration of the compositions and pharmaceutical compositions of the invention is contemplated include, but are not limited to, humans and other primates, mammals including commercially relevant mammals such as non-human primates, cattle, pigs, horses, sheep, cats, and dogs.

The dosage of the above treatments to be administered to a patient will vary with the precise nature of the condition being treated and the recipient of the treatment. The scaling of dosages for human administration can be performed according to art-accepted practices. Strategies for T cell dosing and scheduling have been discussed (Ertl et al, 2011, Cancer Res, 71:3175-81; Junghans, 2010, Journal of Translational Medicine, 8:55).

Pharmaceutical compositions of the present invention may comprise a composition as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present invention are in one embodiment formulated for intravenous administration.

Pharmaceutical compositions of the present invention may be administered in a manner appropriate to the disease to be treated (or prevented). The quantity and frequency of administration will be determined by such factors as the condition of the patient, and the type and severity of the patient's disease, although appropriate dosages may be determined by clinical trials.

When “an immunologically effective amount” or “therapeutic amount” is indicated, the precise amount of the compositions of the present invention to be administered can be determined by a physician with consideration of individual differences in age, weight, extent of infection or metastasis, and condition of the patient (subject). It can generally be stated that a pharmaceutical composition comprising the cells described herein may be administered at a dosage of 10⁴ to 10⁹ cells/kg body weight, in some instances 10⁵ to 10⁶ cells/kg body weight, including all integer values within those ranges. The cell compositions may also be administered multiple times at these dosages. The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease and adjusting the treatment accordingly.

In various embodiments, it may be desired to administer activated cells to a subject and then subsequently redraw blood (or have an apheresis performed), activate cells therefrom according to the present invention, and reinfuse the patient with these activated and expanded cells. This process can be carried out multiple times every few weeks. In various embodiments, cells can be activated from blood draws of from 10 cc to 400 cc. In various embodiments, cells are activated from blood draws of 20 cc, 30 cc, 40 cc, 50 cc, 60 cc, 70 cc, 80 cc, 90 cc, or 100 cc. Not to be bound by theory, using this multiple blood draw/multiple reinfusion protocol, may select out certain populations of cells.

The administration of the subject compositions may be carried out in any convenient manner, including by aerosol inhalation, injection, ingestion, transfusion, implantation or transplantation. The compositions described herein may be administered to a patient transarterially, subcutaneously, intradermally, intranodally, intramedullary, intramuscularly, by intravenous (i.v.) injection, or intraperitoneally. In one embodiment, the T cell compositions of the present invention are administered to a patient by intradermal or subcutaneous injection. In one embodiment, the cell compositions of the present invention are administered by i.v. injection. The compositions of cells may be injected directly into a transplant site, lymph node, or site of infection.

In various embodiments of the present invention, cells activated and expanded using the methods described herein, or other methods known in the art where cells are expanded to therapeutic levels, are administered to a patient in conjunction with (e.g., before, simultaneously or following) any number of relevant treatment modalities including but not limited to treatment with agents such as antiviral therapy, cidofovir and interleukin-2, Cytarabine (also known as ARA-C) or natalizumab treatment for MS patients or efalizumab treatment for psoriasis patients or other treatments for PML patients. In some embodiments, the cells of the invention may be used in a treatment regimen in combination with chemotherapy, radiation, immunosuppressive agents, such as cyclosporin, azathioprine, methotrexate, mycophenolate, and FK506, antibodies, or other immunoablative agents such as CAMPATH, anti-CD3 antibodies or other antibody therapies, cytoxin, fludarabine, cyclosporin, FK506, rapamycin, mycophenolic acid, steroids, FR901228, cytokines, and irradiation. Drugs that inhibit either the calcium dependent phosphatase calcineurin (cyclosporine and FK506) or inhibit the p70S6 kinase that is important for growth factor induced signaling (rapamycin). (Liu et al., Cell 66:807-815, 1991; Henderson et al., Immun. 73:316-321, 1991; Bierer et al., Curr. Opin. Immun. 5:763-773, 1993) can also be used. In one embodiment, the cell compositions of the present invention are administered to a patient in conjunction with (e.g., before, simultaneously or following) bone marrow transplantation, cell ablative therapy using either chemotherapy agents such as, fludarabine, extemal-beam radiation therapy (XRT), cyclophosphamide, or antibodies such as OKT3 or CAMPATH. In one embodiment, the cell compositions of the present invention are administered following B-cell ablative therapy such as agents that react with CD20, e.g., Rituxan.

In one embodiment, subjects may undergo leukapheresis, wherein leukocytes are collected, enriched, or depleted ex vivo to select and/or isolate the cells of interest, e.g., T cells. These cell isolates may be expanded by methods known in the art and treated such that one or more peptide (e.g., SCT) of the invention may be introduced, thereby creating a HLA SCT cell (e.g., HLA SCT immune cell, HLA SCT T cell, HLA SCT B cell, etc.) of the invention. Subjects in need thereof may subsequently undergo standard treatment with high dose chemotherapy followed by peripheral blood stem cell transplantation. In various embodiments, following or concurrent with the transplant, subjects receive an infusion of the expanded HLA SCT cells (e.g., HLA SCT immune cell, HLA SCT T cell, HLA SCT B cell, etc.) of the present invention. In one embodiment, expanded cells are administered before or following surgery.

### EXPERIMENTAL EXAMPLES

The invention is now described with reference to the following Examples. These Examples are provided for the purpose of illustration only and the invention should in no way be construed as being limited to these Examples, but rather should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore and are not to be construed as limiting in any way the remainder of the disclosure.

### Example 1: Decoy HLA-E SCT for Allogeneic Donor Cells

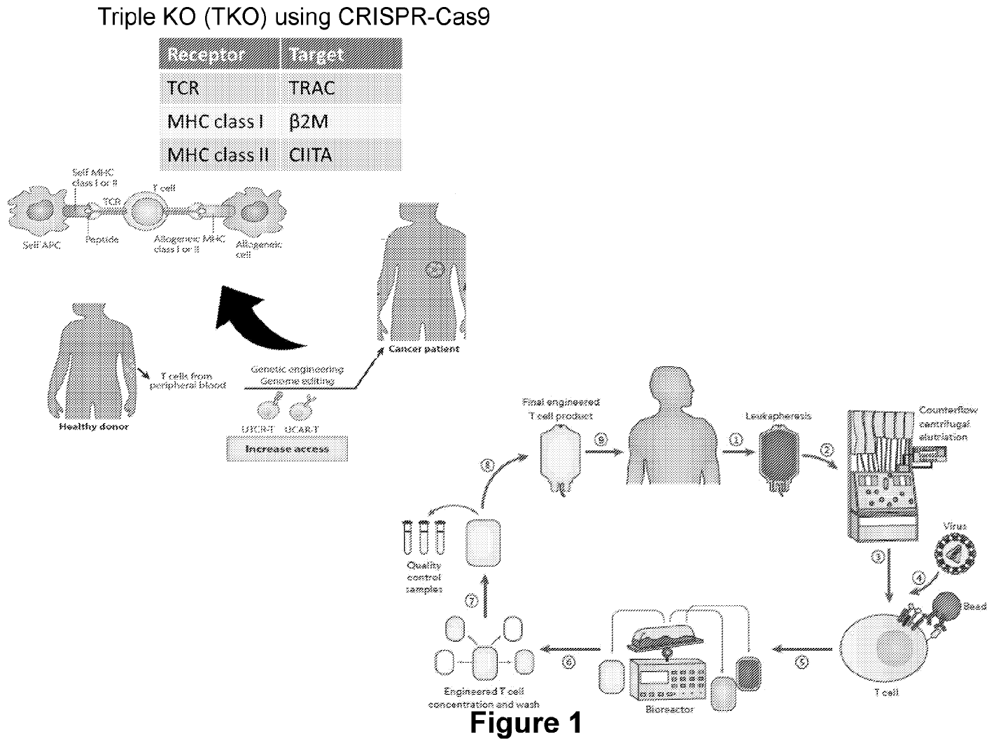

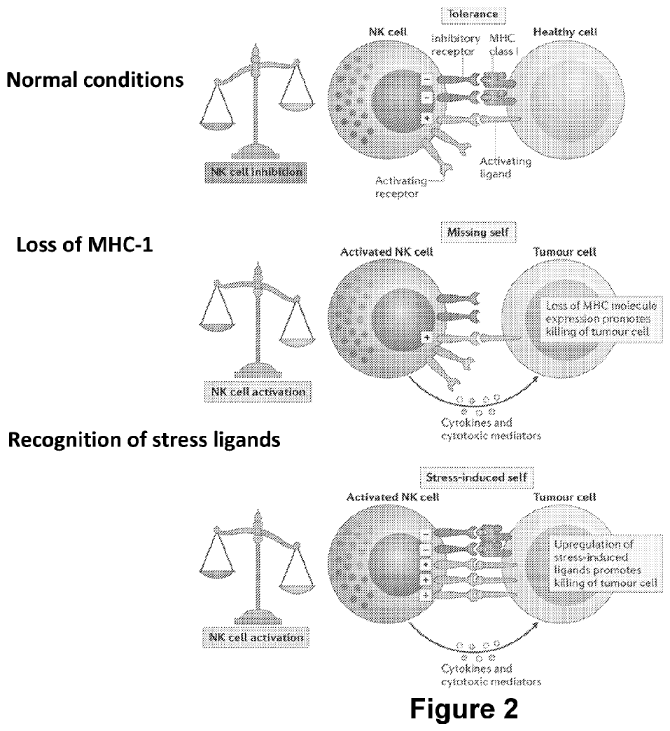

To avoid allo-rejection of the donor CAR T cells by the host, the present studies performed multiple gene edits to render the donor T cells null for Beta-2 microglobulin (B2M), CIITA, and native TCR alpha chain in order to eliminate cell surface MHC class I molecules, MHC class II molecules, and native TCR ab heterodimer. Elimination of cell surface MHC I and MHC II reduces the risk of allo-recognition by the recipient (host) immune system and avoids (or delays) rejection of the transferred donor T cells. Elimination of cell surface native TCR ab heterodimer reduces the risk of graft versus host disease (GvHD), which is mediated by donor T cell recognition of host/recipient MHC molecules and is associated with moderate-severe morbidity and mortality. Genetic editing of B2M, CIITA, and TRAC locus generates triple knockout T cells (TKO) that persist and avoid causing GvHD (FIG. 1). However, it is well established that MHC I deficient cells (of any lineage) are rendered sensitive to NK cells from an unrelated individual and are quickly eliminated after transfer into the unrelated recipient (FIG. 2). KO of MHC-I induces NK cell killing due to “missing self” hypothesis of NK cells (Karre et al., Immunol Today Vol. 11(7), 1990) and NK cells recognize stress ligands, which are upregulated on surface of T cells (Lanier/Spies et al., Science Vol. 285(5428), 1999).

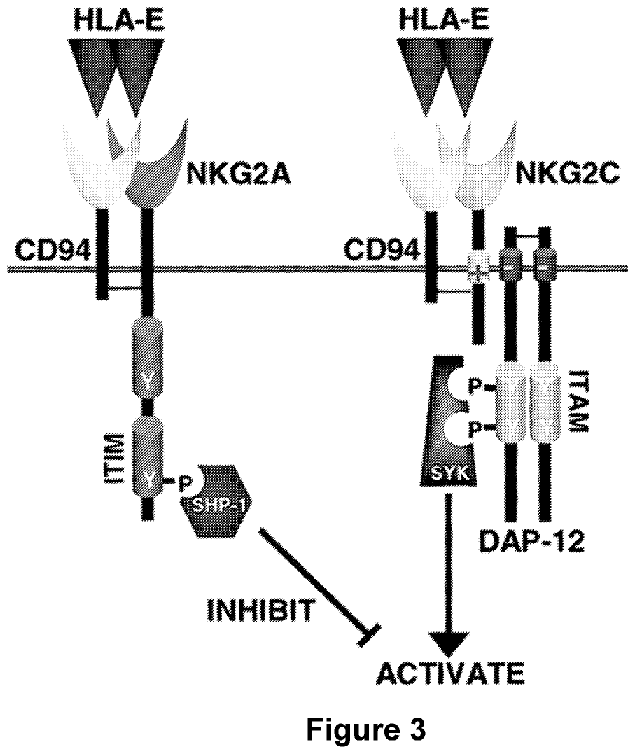

The studies described herein have inserted HLA-E/beta-2-microglobulin/peptide single chain trimer (SCT) into TKO T cells in order to manufacture “Universal” T cells (FIG. 3). The data demonstrated that TKO T cells expressing HLA-E SCT (TKO/E-SCT) avoid recognition by the immune system, specifically NK cells and T cells, and shows improved persistence after transfer into patients. More specifically, expression and interaction of HLA-E with NKG2A prevented the clearance of UCART cells because the interaction between NKG2A and NKG2C is dependent on the peptide that is presented by HLA-E (Rolle et al., Cell Rep Vol. 24, 2018; Llano et al., Eur. J. Immunol. Vol. 28, 1998).

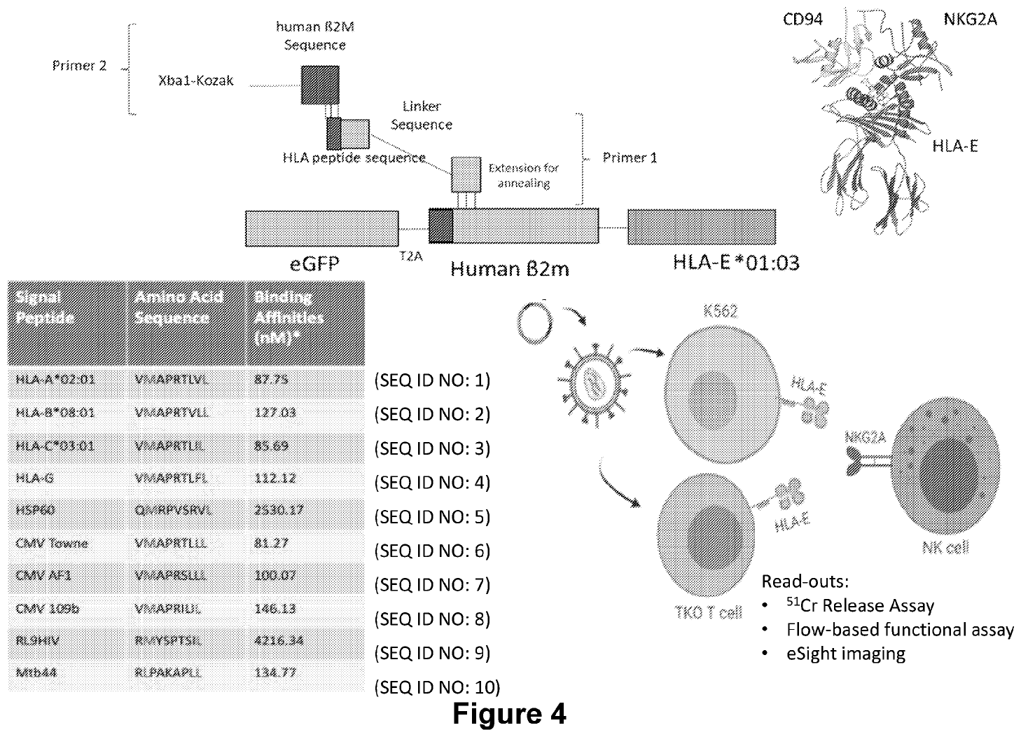

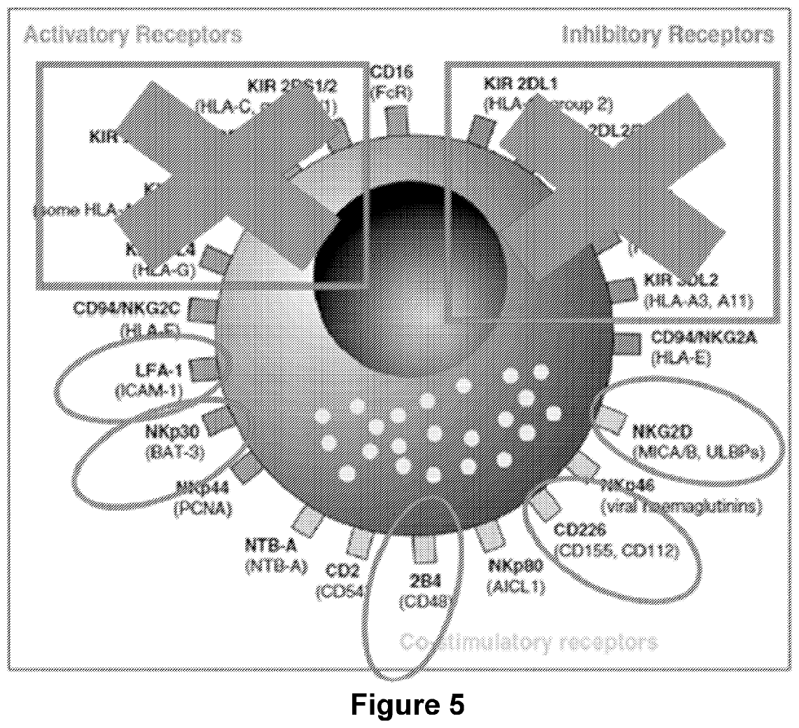

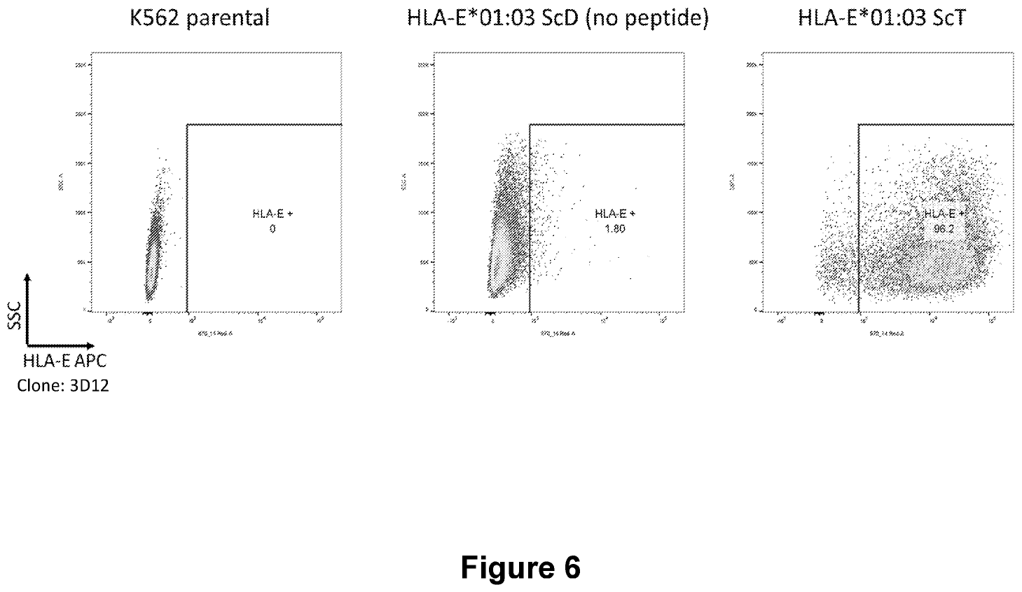

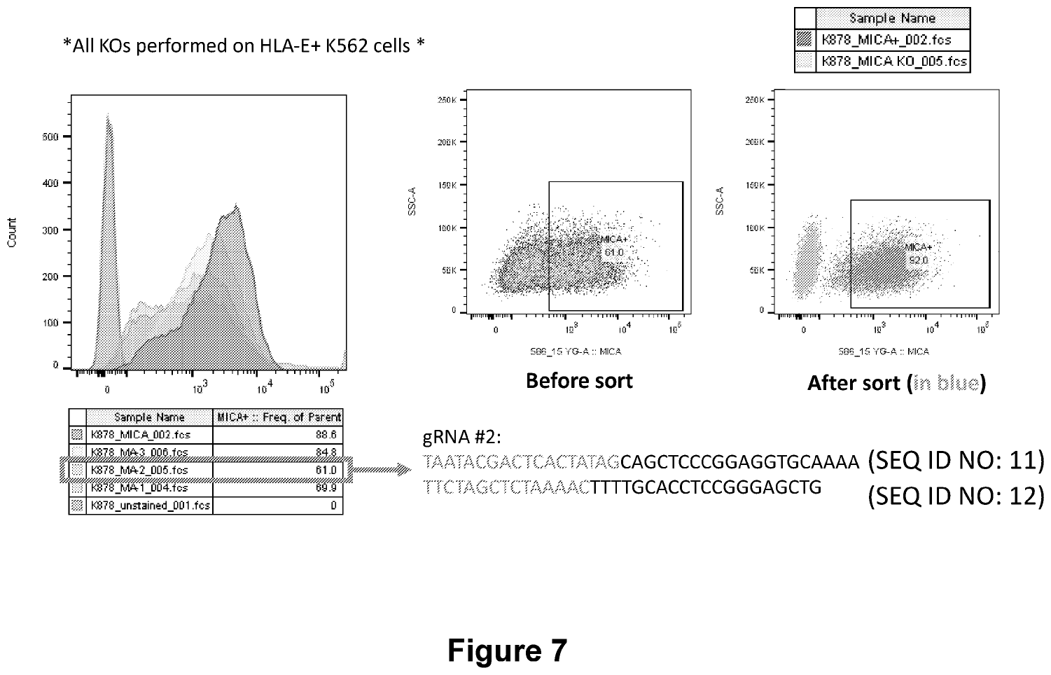

Furthermore, the present studies have utilized the SCT format as a facile platform to generate a new molecular entity that solves a critical gap in the ability to engraft normal donor cells or tissues in an unrelated allogeneic host. As shown in FIG. 4, the identity along with the amino acid sequence and the binding affinity (nM) for HLA-E*01:03 was presented. The present studies designed and tested an HLA-E single-chain trimer (ScT) as an inhibitory receptor for UCART cells and also determined the key NK cell activating receptors that recognize stress ligands on UCART cells (FIG. 5). By LV gene transfer, a panel of K562 transfectants have established to develop a screening platform that permitted functional assays using NK cells as effectors to determine the degree of protection against cytolysis (FIG. 6 through FIG. 9). Expression of HLA-E/beta-2-microglobulin single chain dimer (with no peptide) resulted in low level expression in cells that were devoid of MHC class I indicating that addition of the peptide sequence was required for expression of normal cell surface levels. Next, seven HLA-E SCT constructs expressed in K562 were examined and used as targets in a standard 4 h ⁵¹Chromium release assay. NK cells were used from several normal donors as effector cells to study the ability of HLA-E SCT to act as a decoy to inhibit NK cell mediated killing. It was well-established that both NKG2A and NKG2C bind HLA-E present on the target cell. It was the integration of combined signaling through the two NKG2 receptors that determined NK cell effector function.

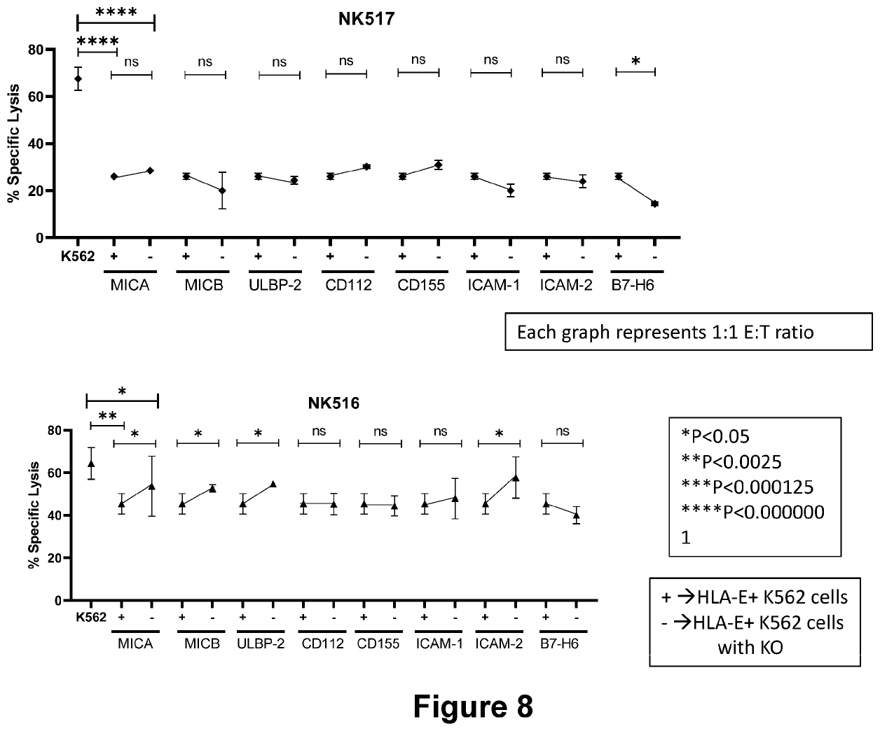

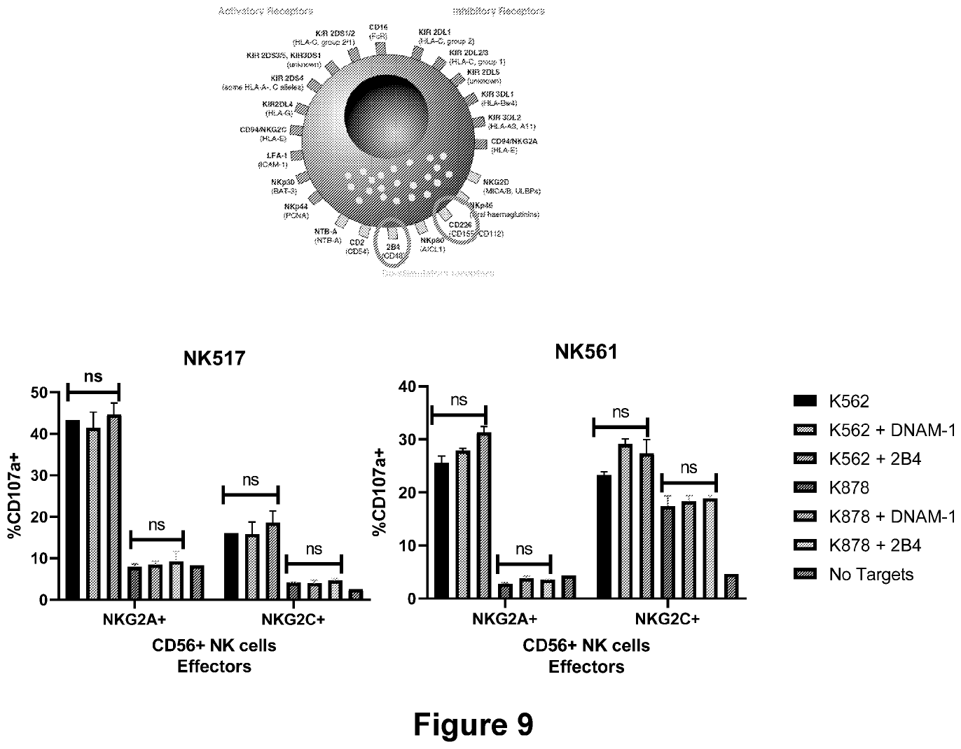

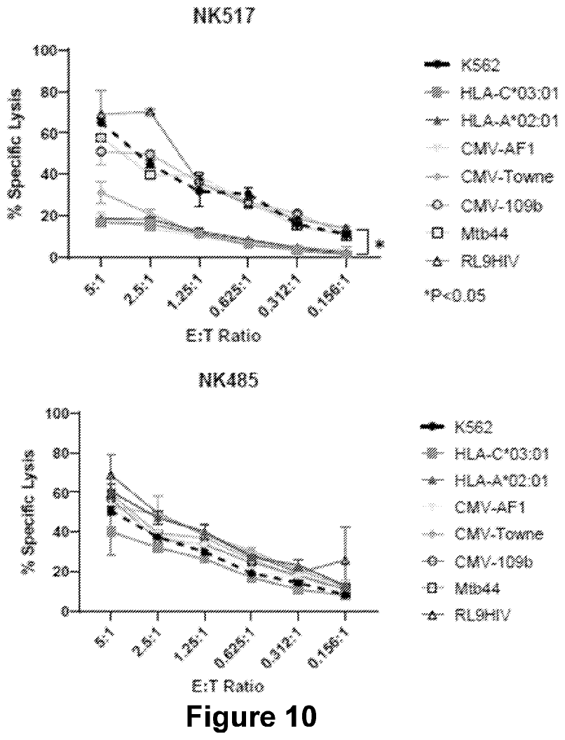

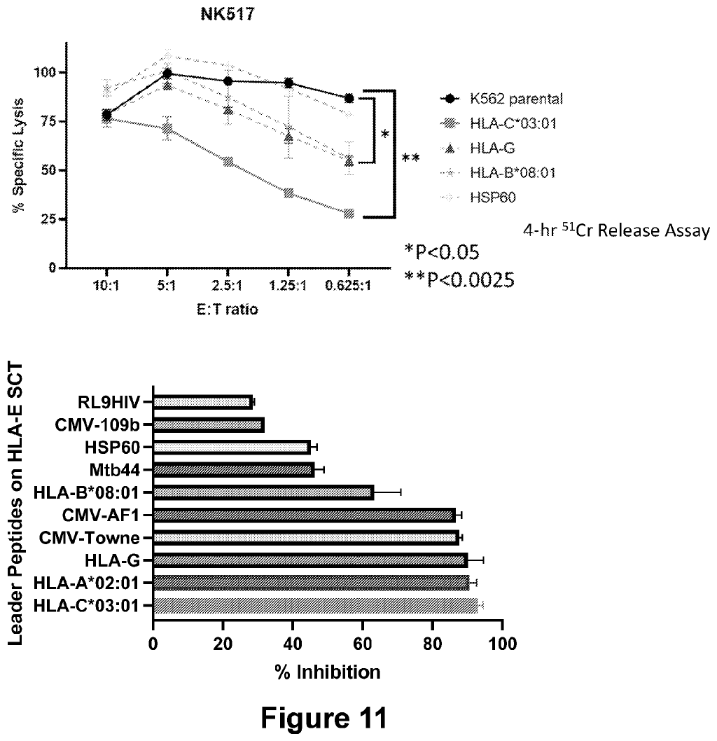

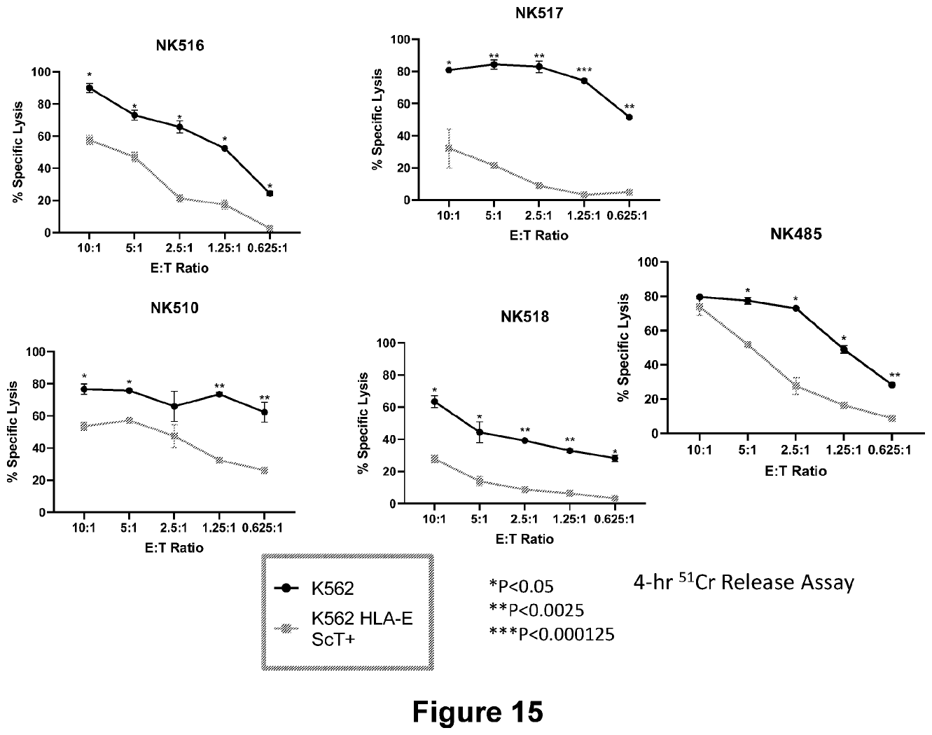

As shown in FIG. 10 and FIG. 11 NK cells from donor 517 were inhibited most significantly by the HLA-C*03.01 leader peptide construct. Inhibition of NK cell lysis was also seen with several other SCT constructs tested; however, the C*0301 leader peptide provided the most reproducible inhibition (FIG. 8 and FIG. 12 through FIG. 15). The experiment was repeated using a larger panel (n=5) of NK cell donors and again, there was significant inhibition of K562 lysis that was mediated by the HLA-E SCT with the C*03:01 leader peptide (aka K878) (FIG. 15).

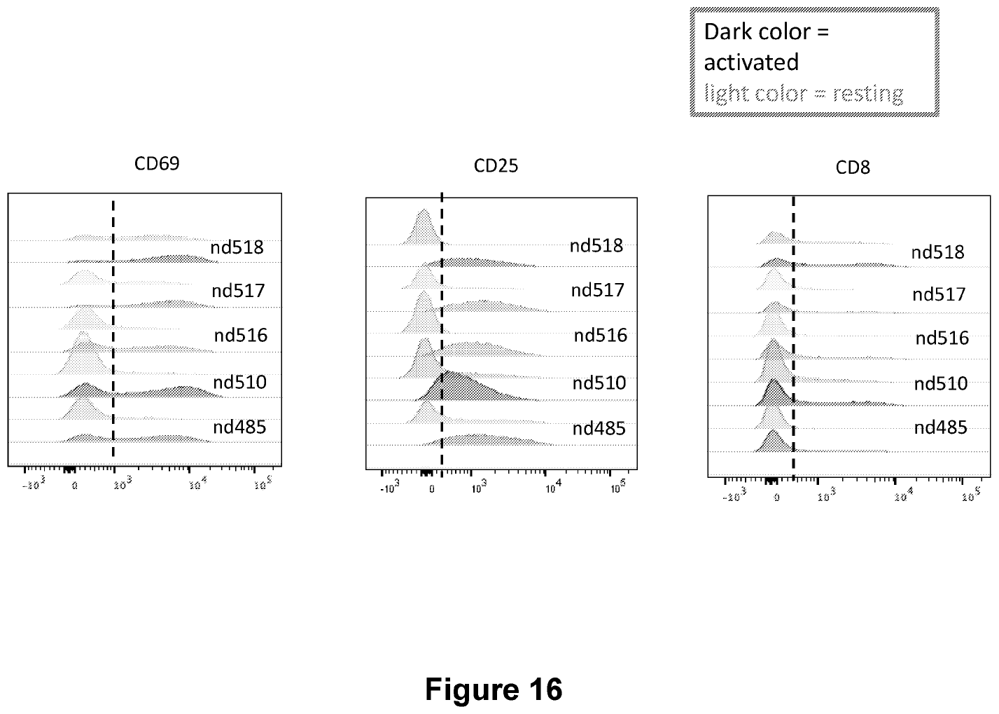

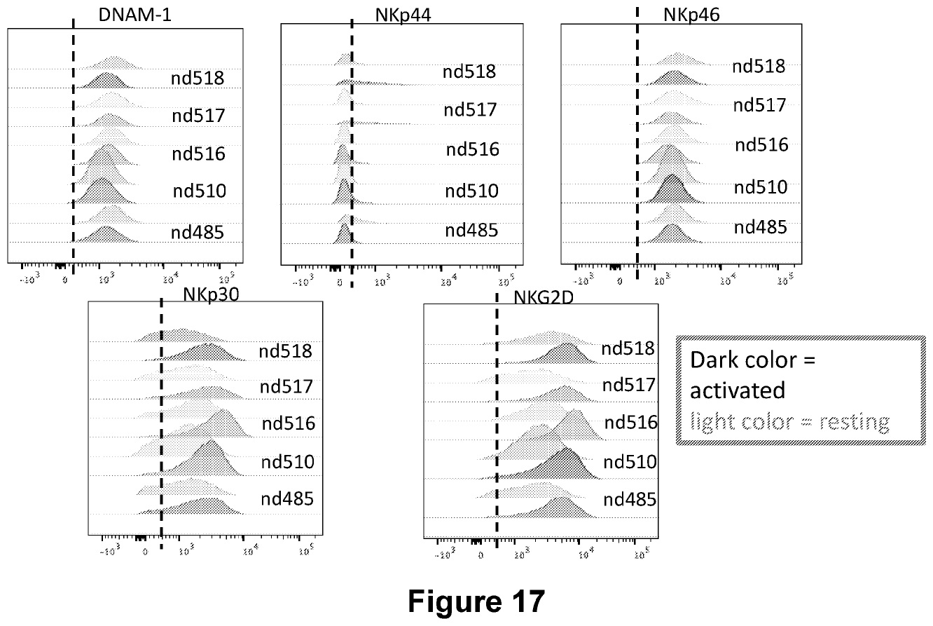

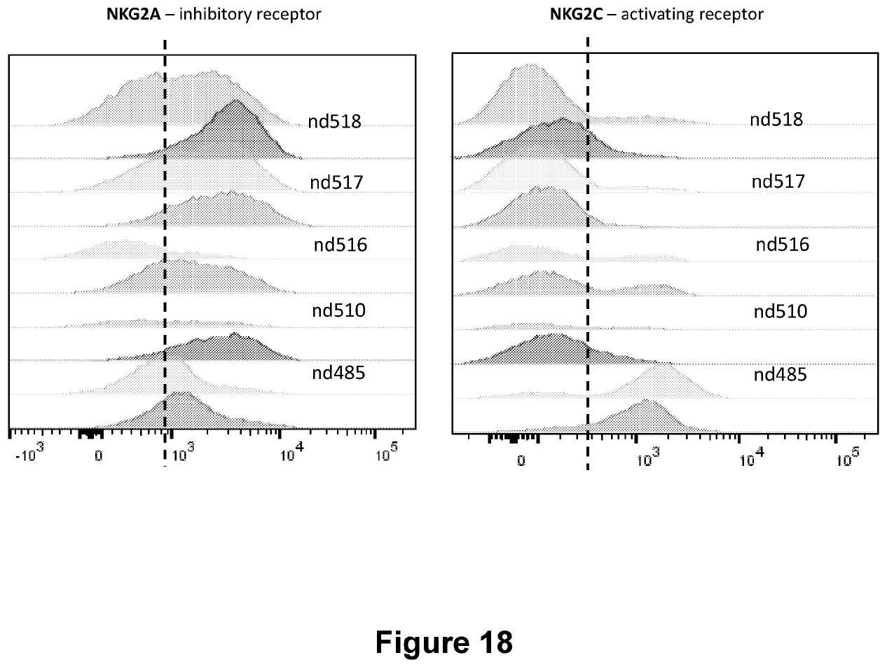

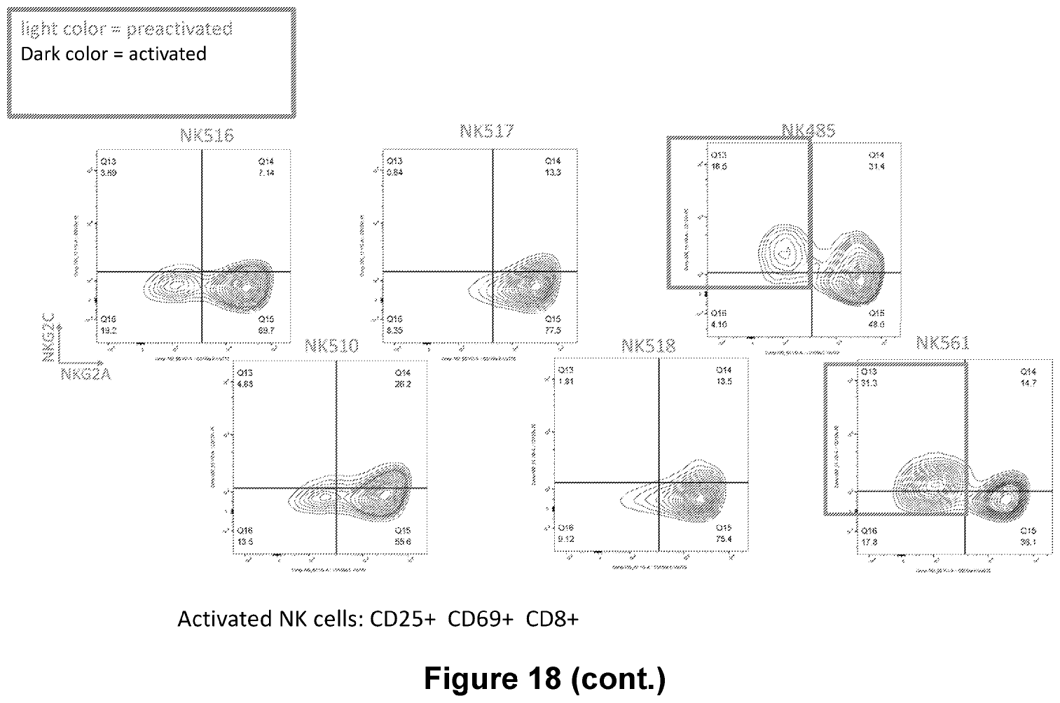

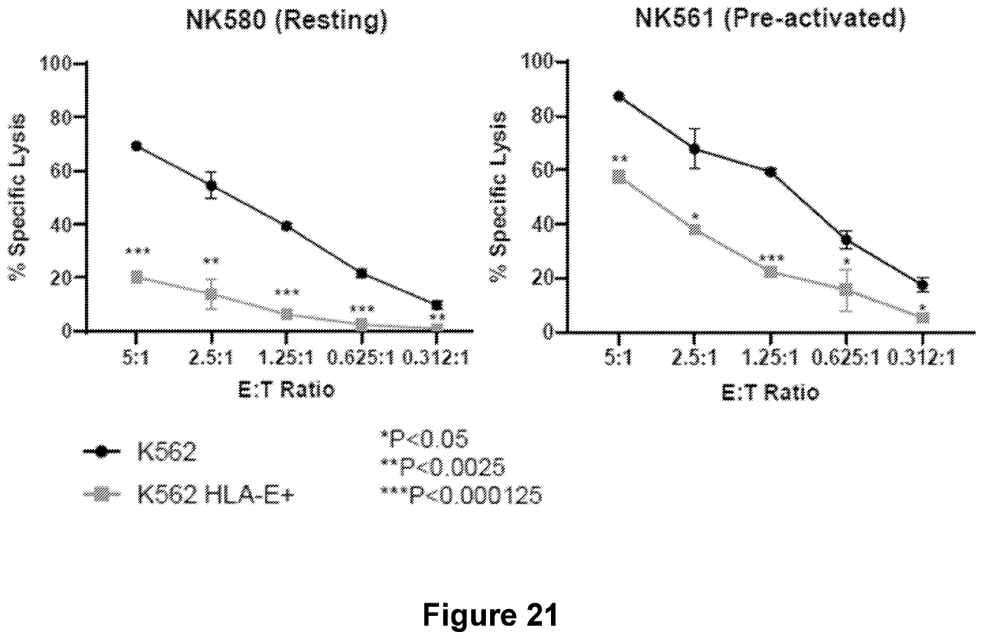

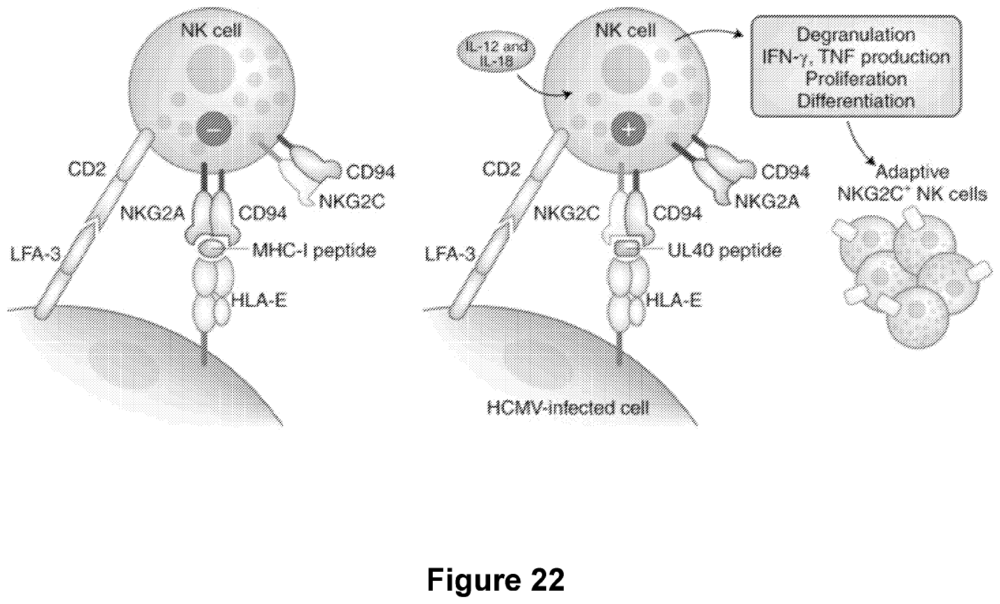

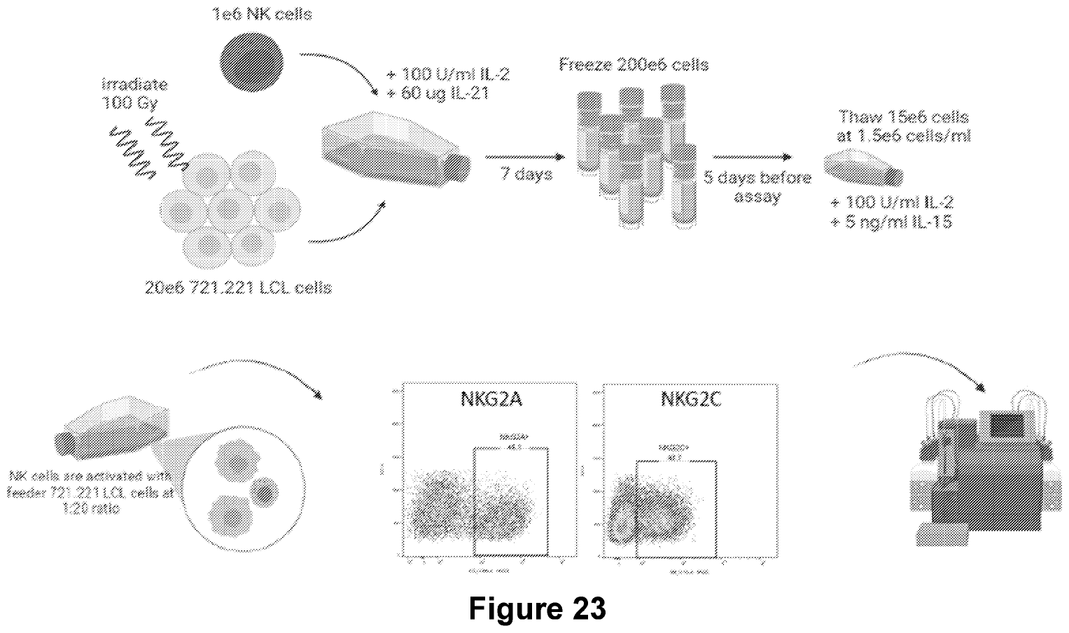

To better understand the differences of NK cell inhibition mediated by K878 (HLA-E SCT), the NKG2A and NKG2C expressions were determined by flow cytometry (FIG. 16 through FIG. 20). NK517 NK cells expressed NKG2A exclusively and NK485 NK cells expressed significant NKG2C as a single population or as a NKG2A+/NKG2C+ double population. NK485 NK cells were also less susceptible to inhibition by the HLA-E SCT. In contrast, NK517 NK cells were almost completely inhibited by HLA-E SCT (FIG. 15 and FIG. 18). Further studies have shown that the NKG2A+/NKG2C+ double positive subset was inhibited by HLA-E SCT, while NKG2C+ single positive NK cells were minimally inhibited by HLA-E SCT. This observation has been repeated on multiple occasions with several donors (FIG. 21 through FIG. 24).

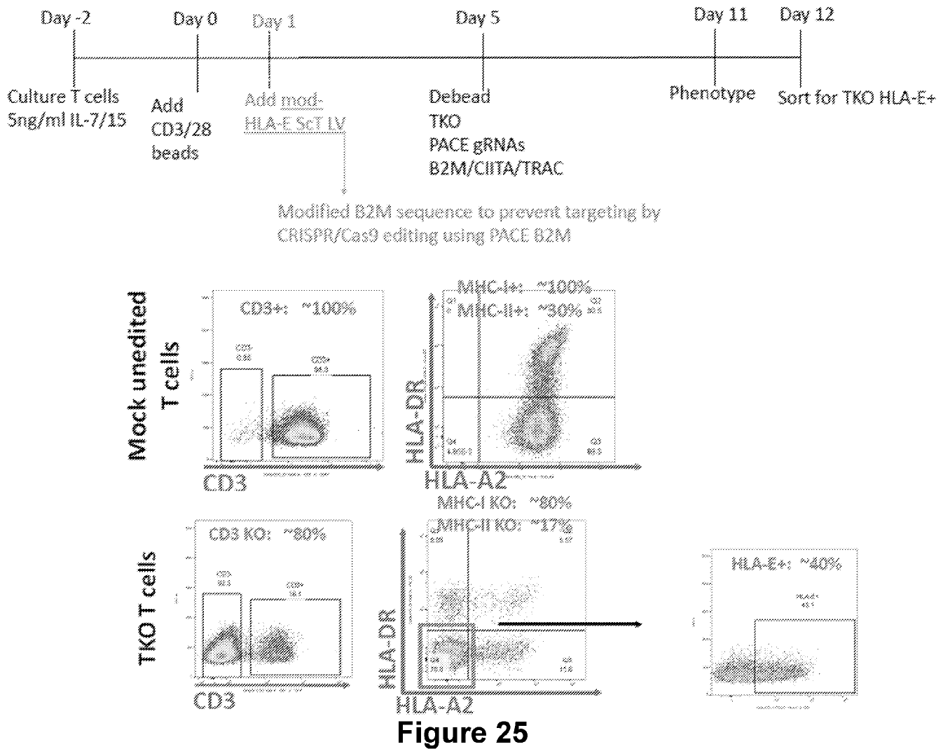

Subsequent experiments used LV gene transfer to express HLA-E SCT encoding the C*03:01 leader peptide in TKO T cells (FIG. 25). Using standard molecular techniques with CRISPR-Cas9, normal donor purified T cells were first activated with anti-CD3/CD28 beads and 24 h later transduced with LV encoding HLA-E*01:03 encoding the C*03:01 leader peptide and placed back into culture. Four days later, the T cells were debeaded and then in the presence of Cas9 ribonucleoprotein (RNP) and sgRNAs for B2M, CIITA, TRAC electroporated using the Maxcyte GTx instrument. Afterwards, the modified T cells were placed back into culture for 7 more days prior to purification using a cell sorter. The purified cells were >99% TKO as assessed by standard flow cytometry.

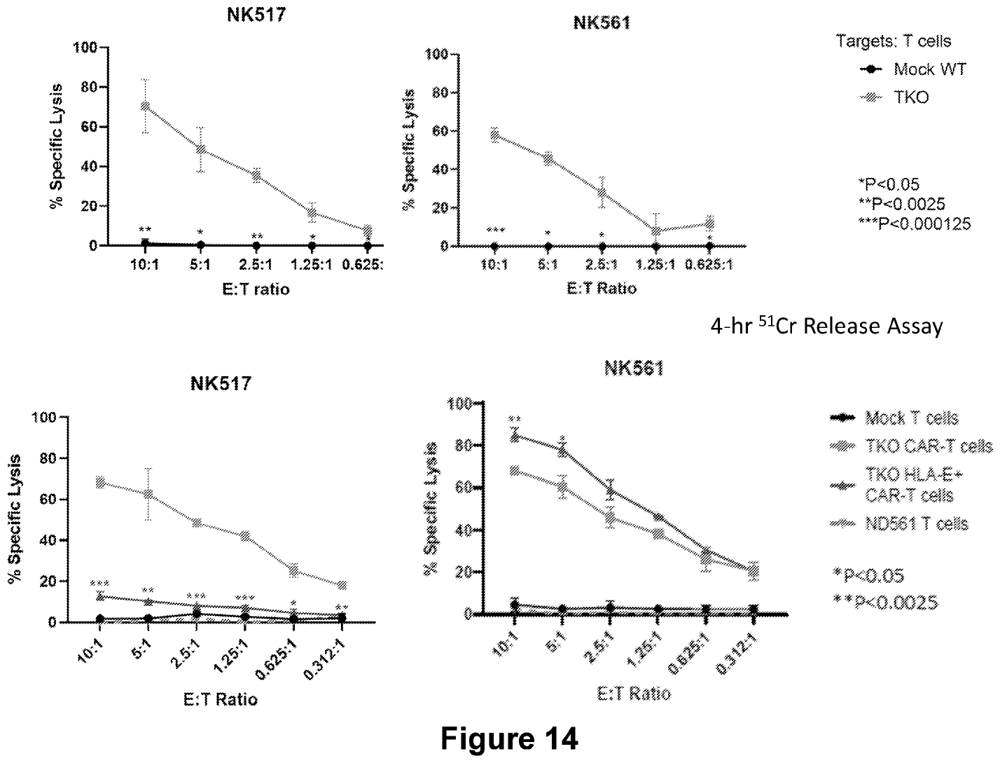

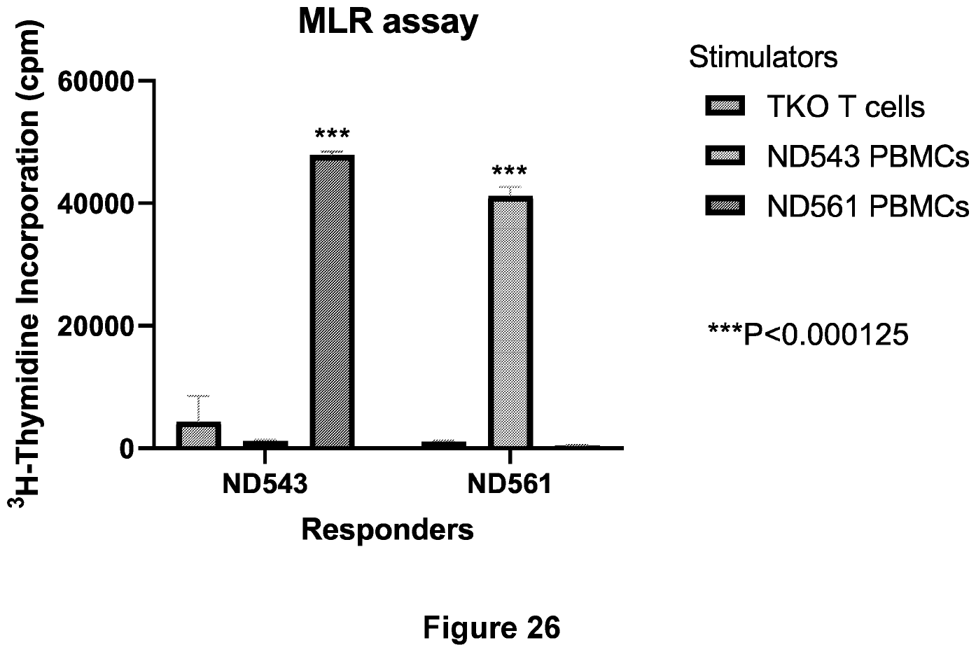

In a representative experiment, the TKO cells (lack MHC-I and MHC-II and CD3/TCR) were susceptible to recognition by NK cells as measured in a standard Cr51 lysis assay. In contrast, the control (mock, WT) T cells which express MHC-I are resistant to lysis as expected (FIG. 14). The TKO cells were next tested in a mixed lymphocyte reaction (MLR test) to determine if the edited TKO cells stimulate normal donors T cells from an unrelated donor (FIG. 26). This is a commonly used test for alloreactivity and is used to assess histocompatibility differences between two individuals. As shown in FIG. 26, two normal donors were examined to assess possible alloreactivity. TKO cells from ND561 and the control autologous PBMC failed to stimulate responder T cells from ND 543, while PBMC561 that were unedited were highly stimulatory as shown by the incorporation of tritiated thymidine in a standard proliferation assay. Likewise, TKO cells from ND543 and the control autologous PBMC failed to stimulate responder T cells from ND561, while the control allogeneic (ND543) PBMC were highly stimulatory. This experiment provided conclusive evidence that TKO cells obtained by standard CRISPR-Cas9 gene editing were non-stimulatory in MLR assays.

Furthermore, subsequent studies further assessed the ability of TKO cells expressing HLA-E SCT to avoid recognition by NK cells. This was a crucial test to determine the efficacy of how HLA-E SCT protects TKO cells from NK cells. TKO cells devoid of MHC-I were very susceptible to NK cell lysis, while control mock/WT (unedited) T cells were much less susceptible to lysis consistent with the prior experiments (FIG. 14). The TKO cells expressing HLA-E SCT were resistant to lysis by NK cells. Thus, TKO cells expressing HLA-E SCT were resistant to attack by allogeneic T cells and by allogeneic NK cells in conventional immunologic assays.

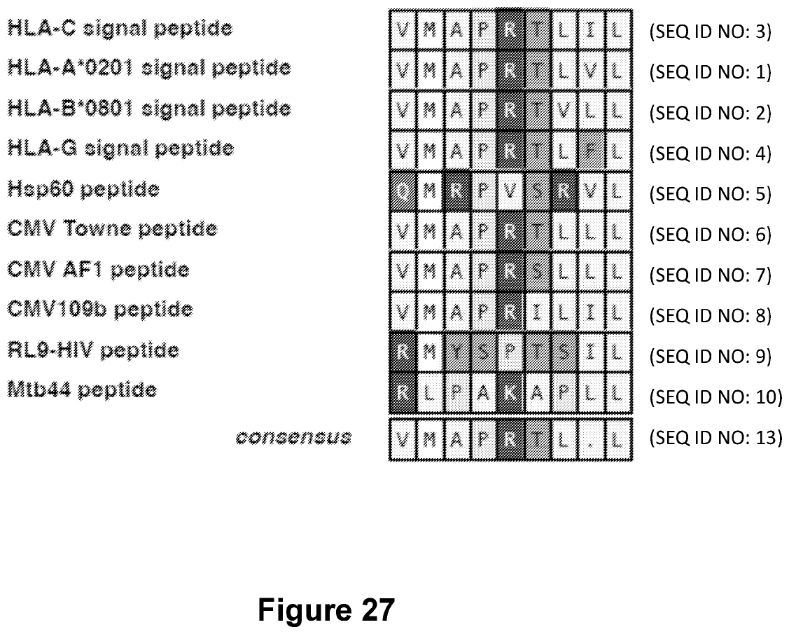

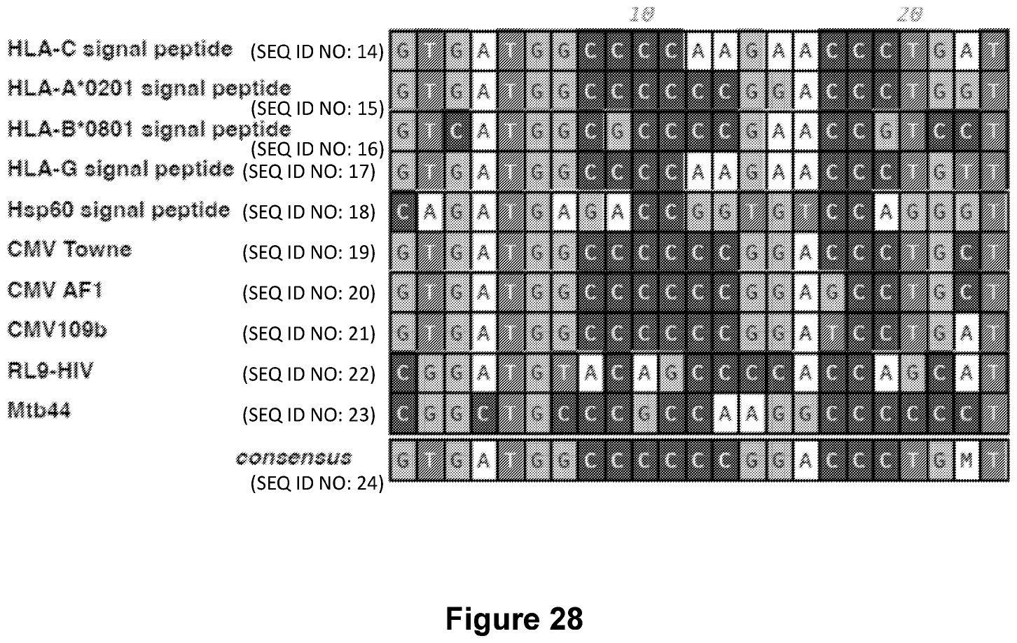

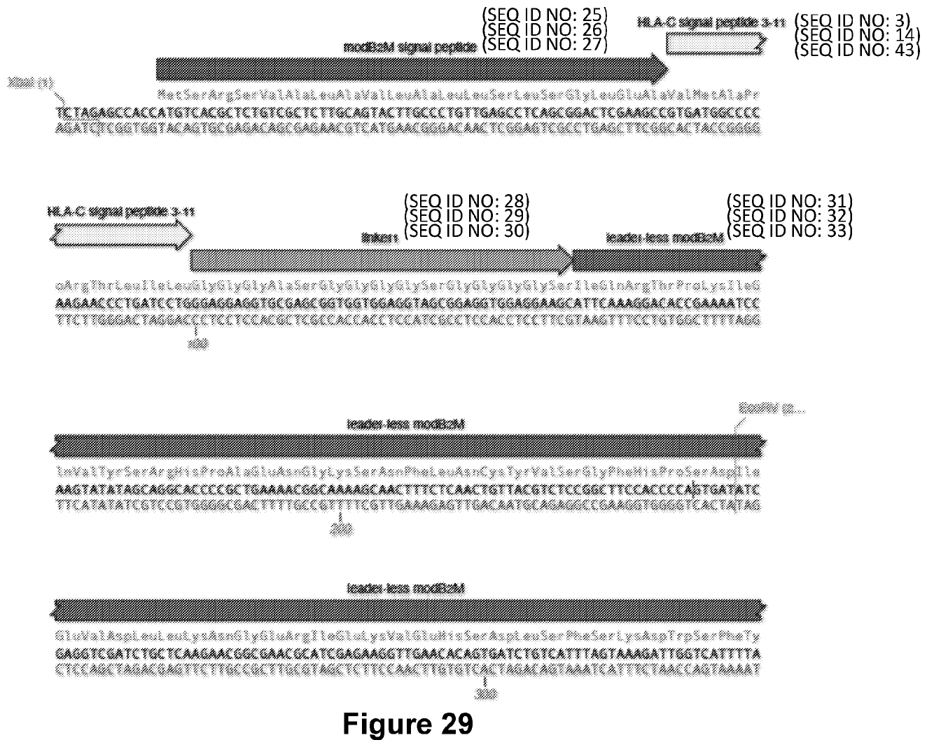

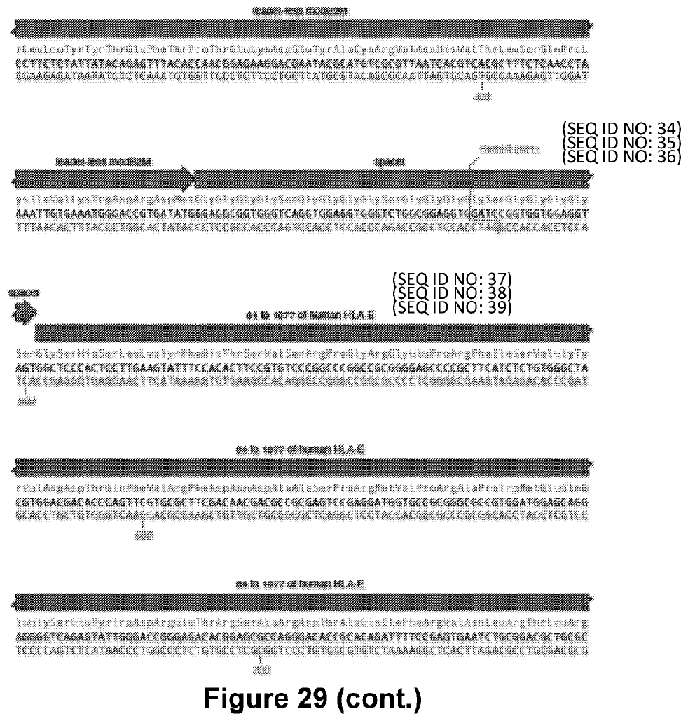

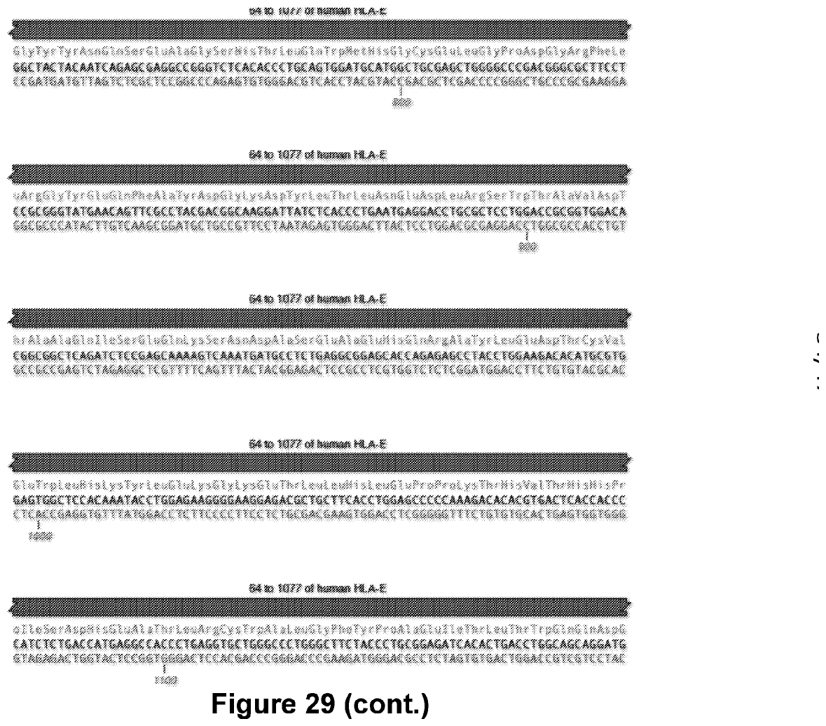

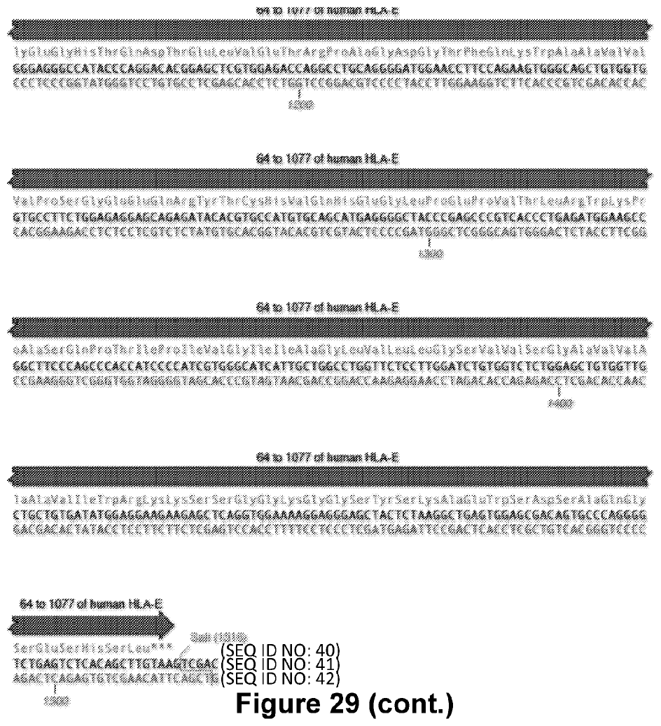

In conclusion, a longstanding problem in biomedical science is the inability to transplant cells, tissues, or organs from one person to another individual without the need for immune-suppression. Allografts and xenografts are rapidly rejected by the host's immune system and the mechanisms involving the innate and adaptive immune system have been delineated in detail. The present studies demonstrated that HLA-E SCT presenting HLA-C leader peptide resulted in significant NK cell inhibition (FIG. 27 through FIG. 29). Furthermore, TKO CAR T cells were targeted for NK cell-mediated killing due to the missing-self response and HLA-E SCT expression on UCART cells inhibited NK cell-mediated killing. Moreover, NK cell populations with higher NKG2C expression were inhibited by HLA-E SCT to a lesser degree than donors with higher NKG2A+ populations. Additional studies focus on evaluation of various stress ligands (i.e., MICA/ULBPs) on TKO cells using standard in vitro assays. In addition, studies also focus on evaluation of TKO/HLA-E SCT/CAR cells in humanized animal models with Nalm-6 leukemia and in-vivo tests with hIL-15 NSG mice to determine in-vivo persistence of UCART cells. Thus, the present studies indicated that the HLA-E SCT protected target cells from NK mediated killing and thus, expression of this novel SCT protected normal donor cells or tissues (that were gene-edited to eliminate MHC molecules) from recognition by the immune system of an unrelated allogeneic host. The HLA-E SCT enable to engineer any cell or tissue or organ that allows transplantation into an unrelated host, such as another human being.

The materials and methods employed in these experiments are now described.

### Standard 4 h ⁵¹Chromium Release Assay

NK cells from normal donor 517 were expanded for 10 d in IL-2/IL-15 and used as effector cells at the indicated effector to target ratios (E:T ratio) in a standard 4 hr ⁵¹Cr release assay. Target cells (10,000 per well) studied included K562 parental and K562 transfected with various HLA-E single chain trimer constructs. Target cells were labeled with ⁵¹Chromium for 1 h, washed 3×, and then plated in 96 well trays containing NK cells. All assays are performed in triplicate. After 4 h incubation at 37° C., 5% CO₂, the plate was removed from the incubator and centrifuged for 10 minutes. 50 ul of supernatant was carefully removed and counted in a MicroBeta2 LumiJET microplate counter (Perkin Elmer). Data were represented as percent-specific lysis reported as mean+/−SD. Percent inhibition of lysis for each SCT was calculated based on CPM obtained for maximum release (SDS) values and demonstrated that leader peptides expressed by the various SCTs influence the inhibitory function of these molecules in NK activity (FIG. 11).

### Real Time Apoptotic Cell Death Analysis

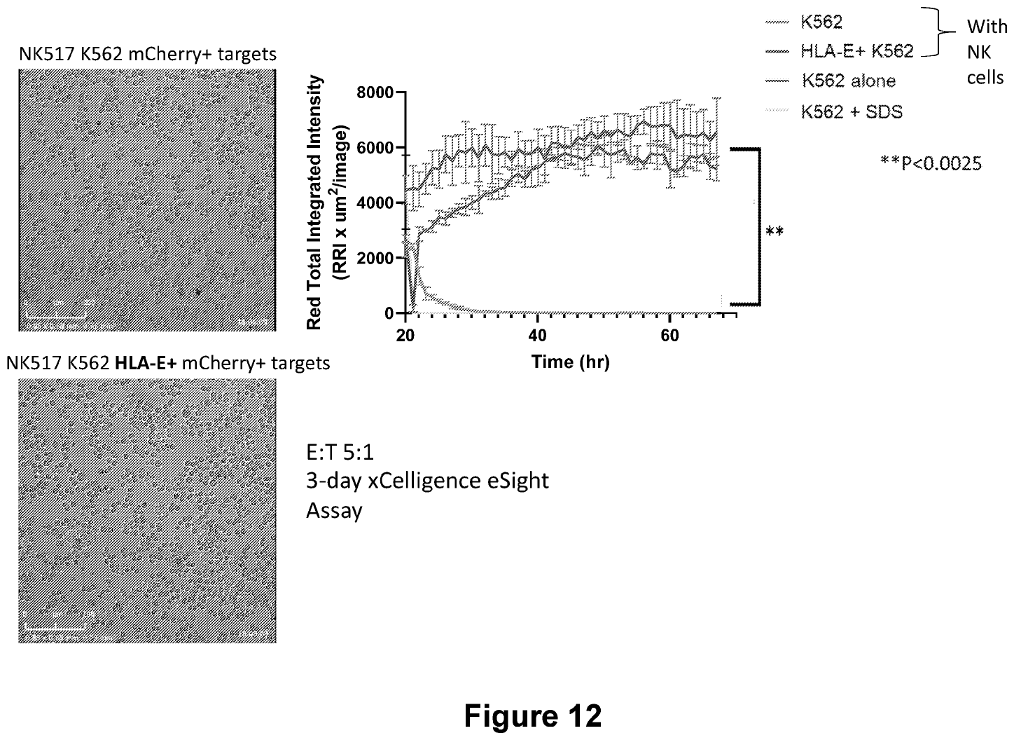

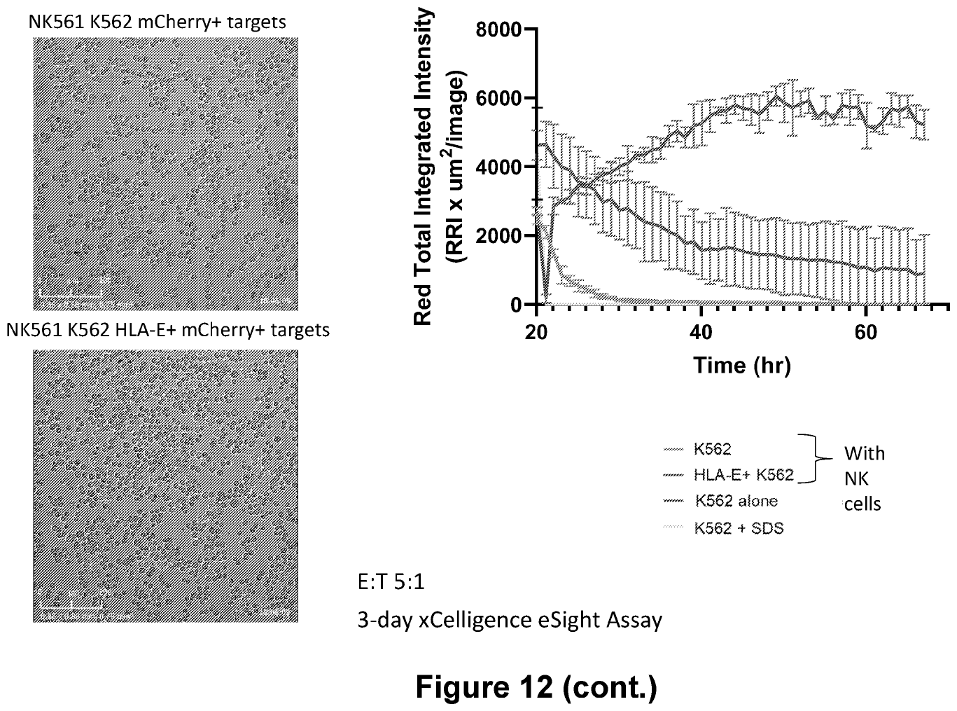

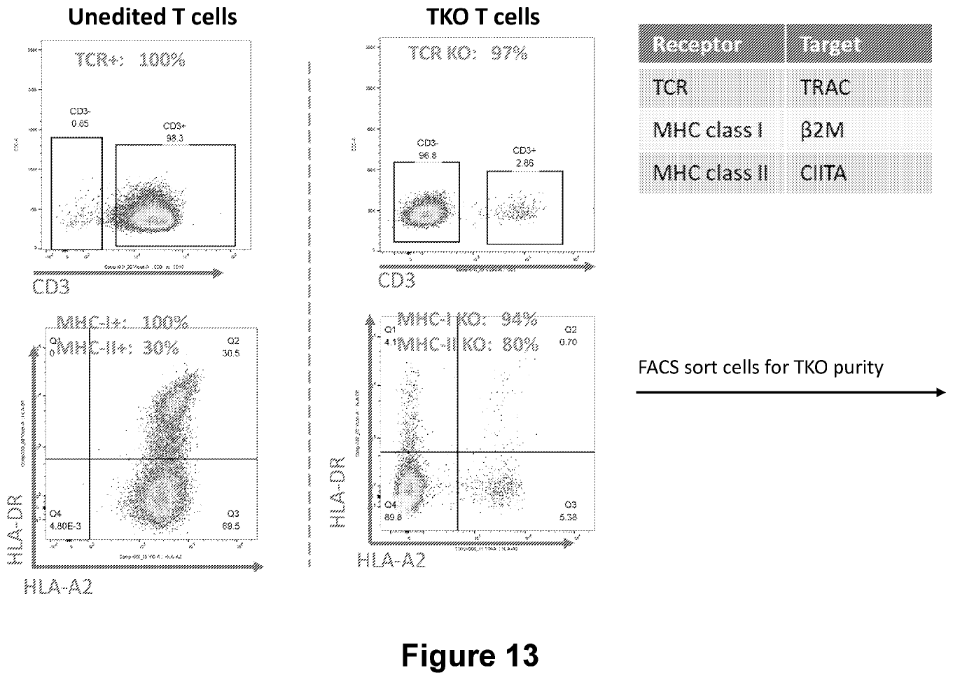

Real time apoptotic cell death analysis (live cell imaging with cellular impedance) was performed using the xCELLigence real time cell analysis eSIGHT system (Agilent). mCherry labeled-K562 target cells were plated (10,000/well) and allowed to adhere for 24 h. Effector NK cells (normal donor 517) were added at a 5:1 E:T ratio and the assay was continued for 3 additional days. Red total integrated intensity (live imaging) was measured every 15 min for 3 days. Decreased in intensity reflected elimination of targets. Killing of K562 parental cells was evident (red line), while K562 cells expressing the HLA-C*03:01 leader peptide/HLA-E SCT (aka K878) were protected and survive in the presence of NK517 (FIG. 12). Parental K562 cells alone (no NK) also exhibited growth (control), while K562 cells died in the presence of SDS (control). Values reflected the mean of triplicate wells+/−SD.

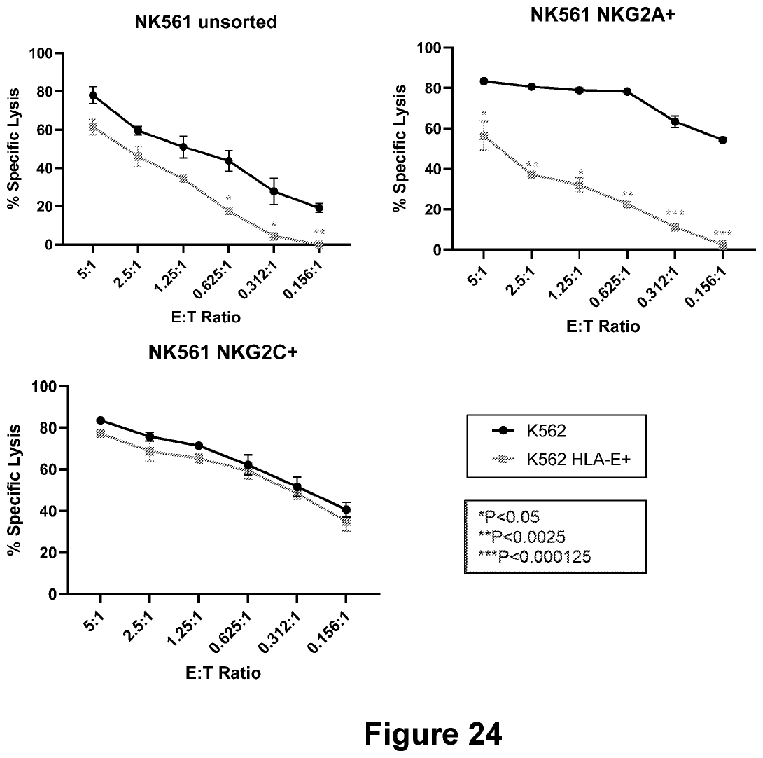

HLA-E SCT provided partial protection from killing by ND561 NK cells (21% NKG2C+) in the 3 d live cell imaging (xCELLigence) assay. This data contrasted with that observed for ND517 (primarily all NKs were NKG2A) were complete lysis protection by SCT was observed (FIG. 12). The balance of NK populations (NKG2A vs. NKG2C) within a donor influences the functional outcome upon SCT engagement by NK cells (FIG. 24).

### TKO Edited T Cells

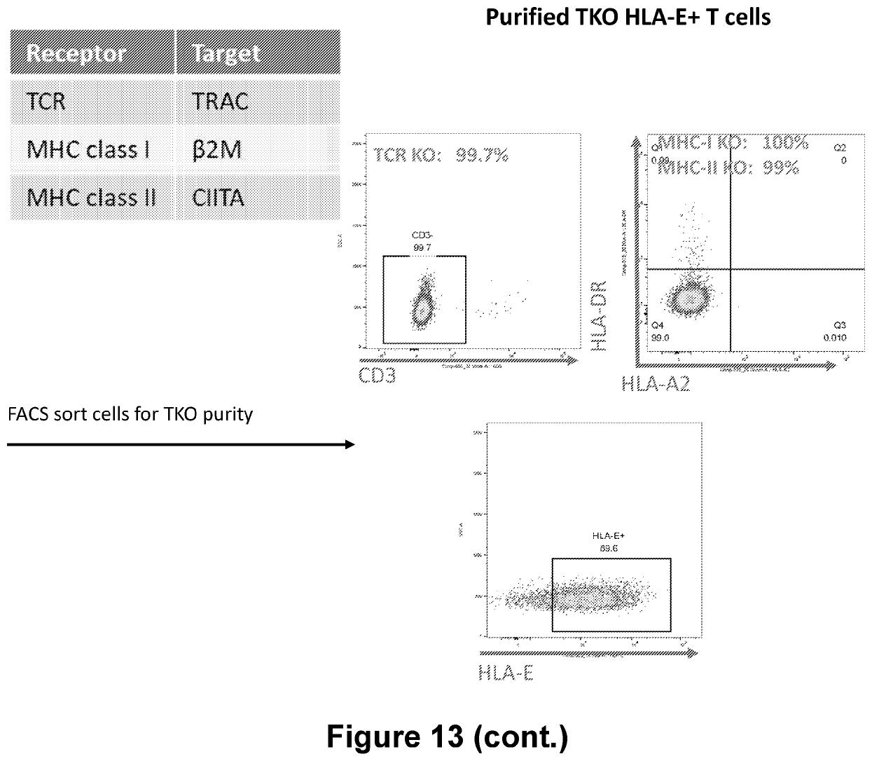

Normal donor T cells were activated with anti-CD3/CD28 beads (Dynal) under standard conditions. After 5 d, cells were gene-edited using sgRNAs for TRAC, B2M, and CIITA by electroporation (Maxcyte GTx) in the presence of Cas-9 RNP as described. In some experiments, the Triple knock-out (TKO) edited T cells were transduced with lentivirus encoding HLA-E SCT (C*03:01 leader peptide) and maintained in media containing IL-7/IL-15. Unedited ND T cells served as the control. Phenotyping was performed on d 10 and analyzed by flow cytometry (FIG. 13).

### Expression of HLA-E on TKO T Cells

TKO T cells were sensitive to NK mediated lysis and were protected by expression of HLA-E SCT. NK517 effector T cells were used in 4 h ⁵¹Cr release assays to examine the protection mediated by overexpression of HLA-E SCT in normal donor TKO T cells. As a control, unedited (mock) allogeneic T cells were resistant to NK cell lysis as were autologous mock (ND517) T cells. TKO CAR-T cells with the HLA-E SCT were tested and shown to be resistant to NK cell mediated killing. Control TKO CAR-T cells (red line) were sensitive to NK cell lysis (FIG. 14).

NK cells from five normal donors were studied in a 4 h ⁵¹Cr release assay using K562 HLA-E SCT targets. Consistent protection was seen across all five donors (FIG. 15). As a control, parental K562 cells were killed by NK cells from all donors.

### NKG2A/NKG2C Expression

Some donors expressed NKG2C that is a known activating receptor, which binds HLA-E on target cells. It has been reported that CMV seronegative individuals have low levels (1-2%) of NKG2C+ NK cells, while a subset (about 1/3) have significant numbers of NKG2C+ NK cells (FIG. 18).

### NK Cell Activation by K562 Cells+/−HLA-E SCT

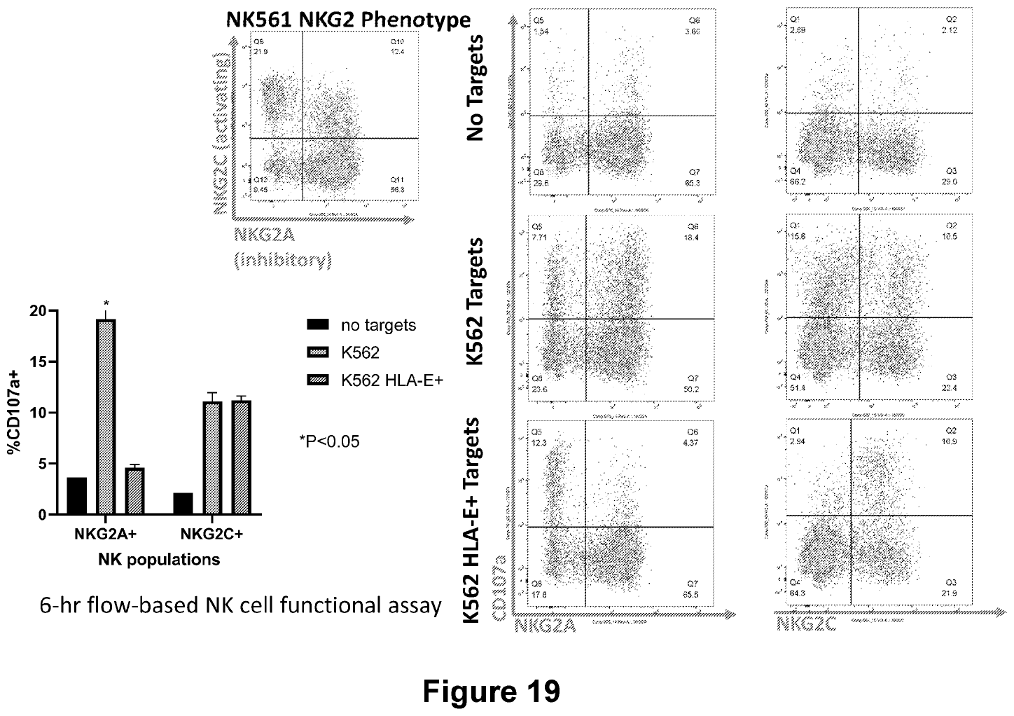

Flow based assay was utilized to examine NK cell activation by K562 cells+/−HLA-E SCT. NK cells were mixed with K562 cells (1:1 ratio) for 6 h and then stained with antibodies to CD107a to measure degranulation of the major subsets (NKG2A+ and NKG2C+). The bar graph summarized the representative results that indicated the protection provided by HLA-E SCT for the NKG2A+ subset (FIG. 19). In contrast, HLA-E SCT conferred no protection of K562 by NKG2C+ subset.

### Stress Ligands Evaluation

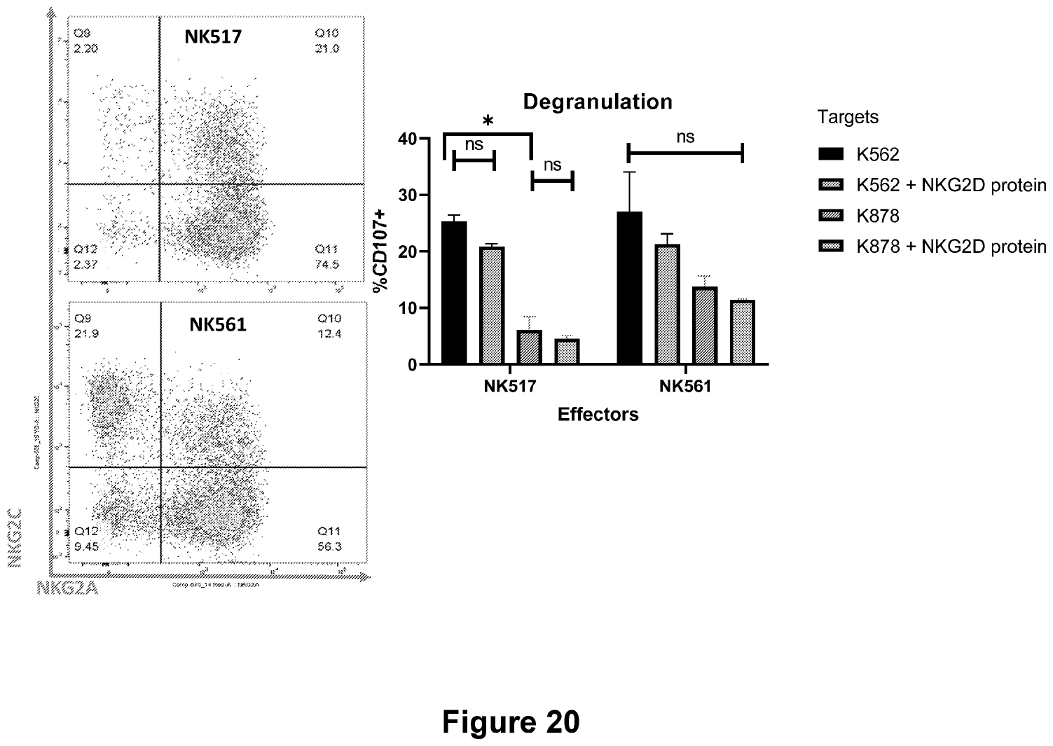

Soluble NKG2D protein was employed to block stress ligands (MICA, MICB, ULBP 1-6) on K562 target cells, which interfered with NKG2D mediated recognition of target cells. The data demonstrated that NKG2D protein had minimal efficacy for parental K562 and K878 (HLA-E SCT) target cell recognition by NK cells from two donors (FIG. 20).

### Example 2: Determining the Histocompatibility Barriers Between Universal CAR T (UCART) Cells and NK Cells

CAR T cell adoptive therapies have proven efficacy in the treatment of hematological malignancies. However, there are challenges to the current CAR-T cell manufacturing process, one being manufacturing failure due to dysfunctional, patient-derived T cells (Thommen et al., 2018, Cancer Cell, 33:547-562). A proposed solution to overcoming this limitation is the development of universal CAR-T (UCART) cells engineered from healthy, normal donor-derived T cells. Histocompatibility barriers must be addressed when creating the UCART cell. Ablation of the alpha/beta TCR surface expression prevents graft-versus-host disease while host-versus-graft responses are mitigated by ablating the surface expression of the major histocompatibility complex (MHC) class I and II molecules (Abdelhakim et al., 2017, Biomedicines; Wang et al., 2015, Stem Cells Transl Med., 4:1234-1245). These triple knockout (TKO) T cells serve as universal recipients for development of the UCART cell.

Importantly, a consequence of MHC class I ablation is NK cell activation due to the “missing-self” response (Ljunggren et al., 1990, Immunol Today, 11:237-244). HLA-E, a non-classical MHC class I molecule, is known to interact with the NK cell inhibitory receptor NKG2A and its overexpression on UCART cells was herein shown to mitigate NK cell activation. Because the HLA-E/NKG2A interaction is dependent upon the peptide presented by HLA-E, 10 HLA-E single-chain trimer (SCT) constructs expressing various peptide sequences were created to determine which HLA-E/peptide complex would result in significant NK cell inhibition (Borrego et al., 1998, J Exp Med 1998, 187:813-818). Ten HLA-E single-chain trimer (SCT) constructs, each encoding and presenting a different signal peptide, were created for cell surface expression on K562 cells. HLA-E+ K562 cells were used in preliminary experiments before moving the lead candidate HLA-E construct into TKO T cells. Functional assays were conducted using primary human NK cells.

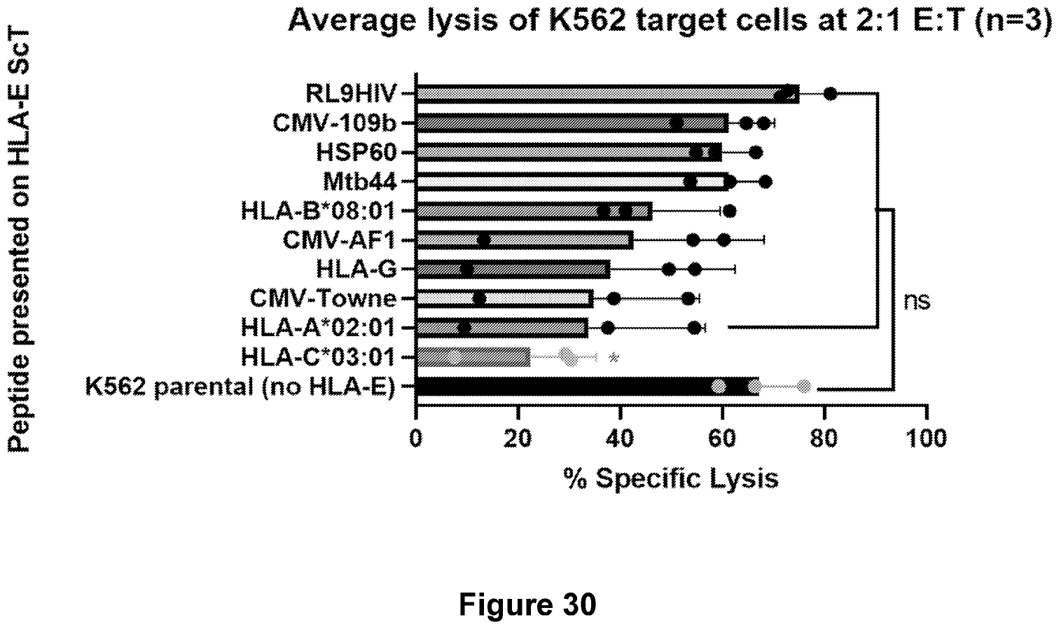

K562 cells transduced to express these HLA-E/peptide complexes were used as a model for UCART cells in preliminary experiments. An HLA-E SCT presenting an HLA-C leader peptide (HLA-E/C) resulted in significant inhibition of NK cells as determined by flow cytometry-based NK cell degranulation (CD107a) assays. Decreased lysis of K562 HLA-E/C-expressing target cells in ⁵¹Cr release assays further validated the inhibitory effect of HLA-E/C complexes on NK cell activation (FIG. 12 and FIG. 30 through FIG. 35). More specifically, FIG. 30 depicts representative results of a 4-hour ⁵¹Cr release assay demonstrating HLA-E presenting HLA-C*03:01 leader peptide resulted in the most significant reduction in K562 lysis indicating its ability to inhibit NK cell activation due to the “missing-self” response. Shown at 2:1 E:T ratio.

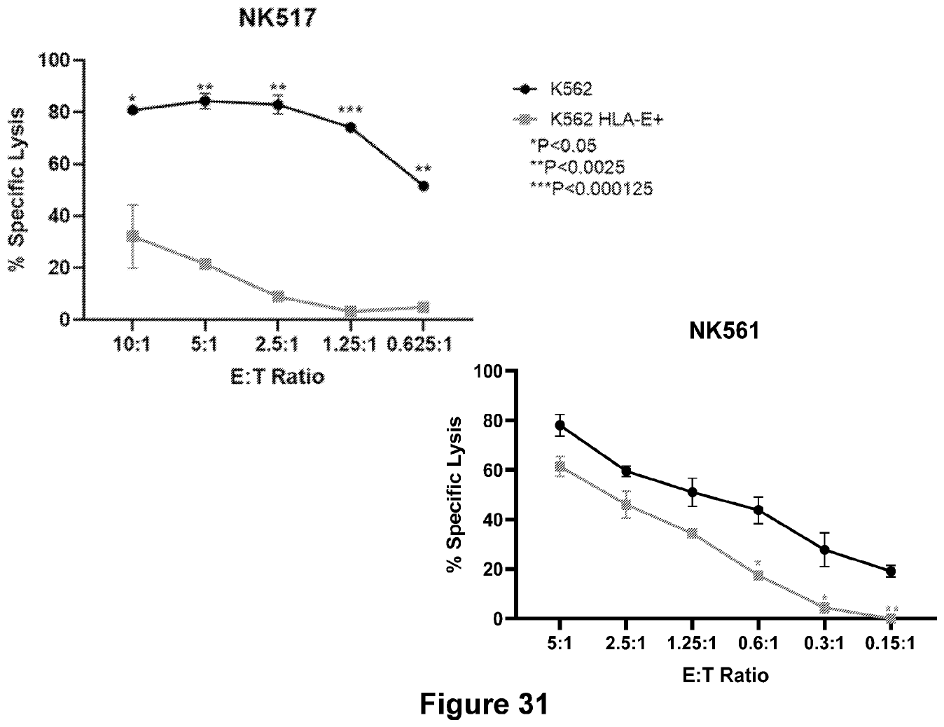

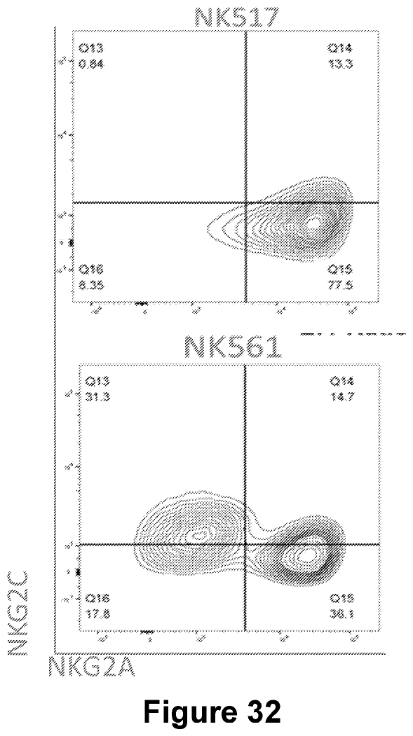

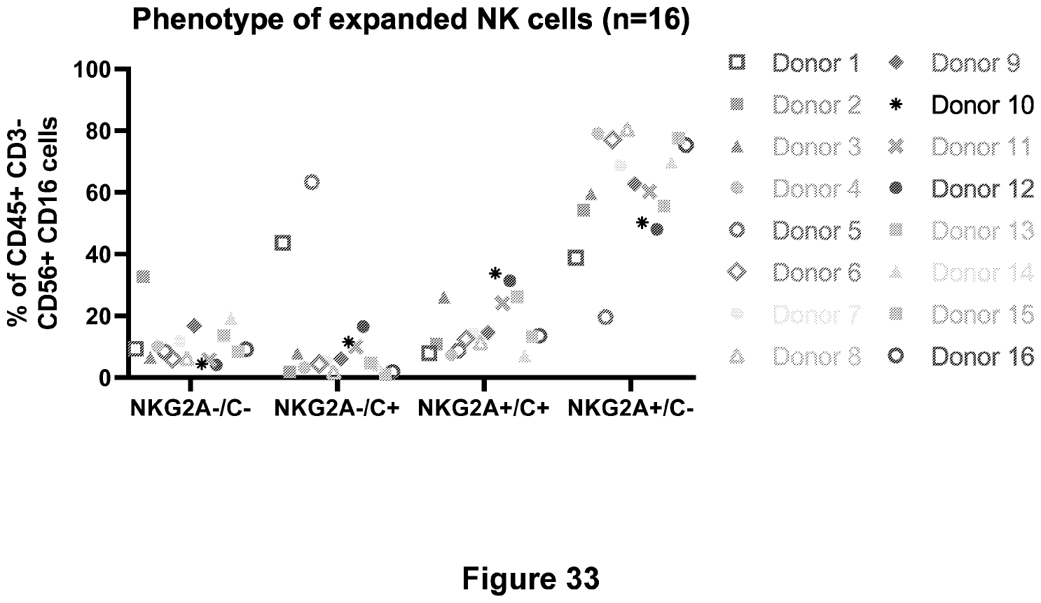

FIG. 31 further depicts representative results demonstrating HLA-E+ K562 cells inhibited NK cells from different donors and effect depended on phenotypic differences of donor NK cell populations and FIG. 32 depicts representative results demonstrating surface expression of NKG2A and NKG2C of donors shown in FIG. 31. FIG. 33 depicts representative results demonstrating NKG2A and NKG2C surface expression of expanded NK cells form 16 different normal donors.

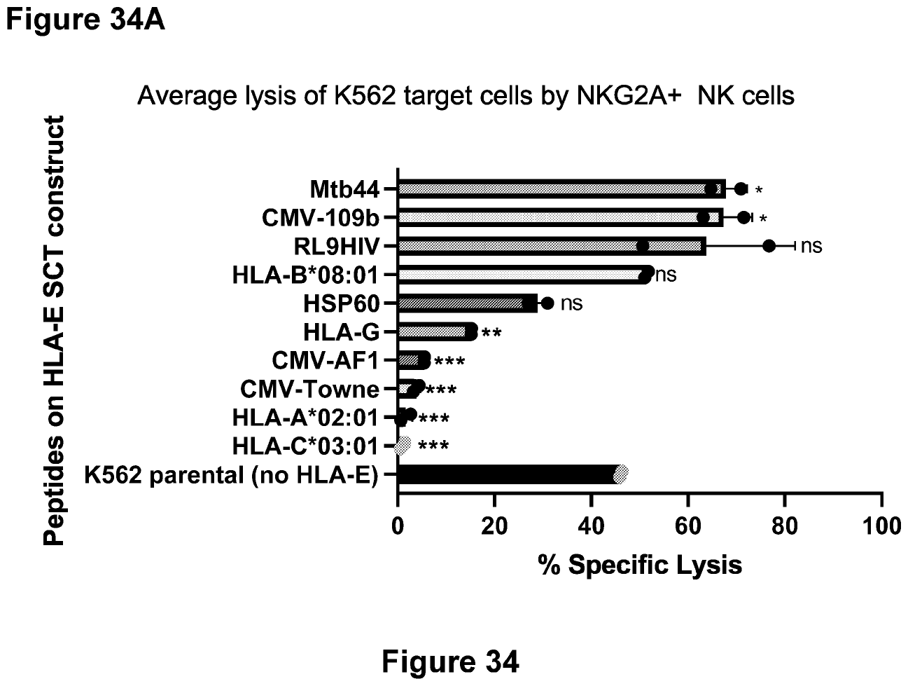

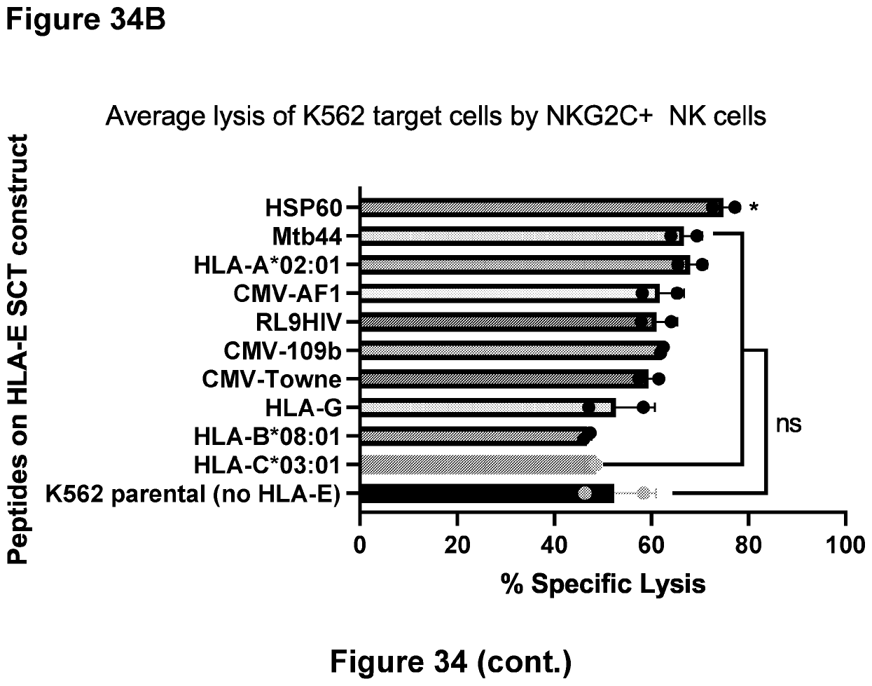

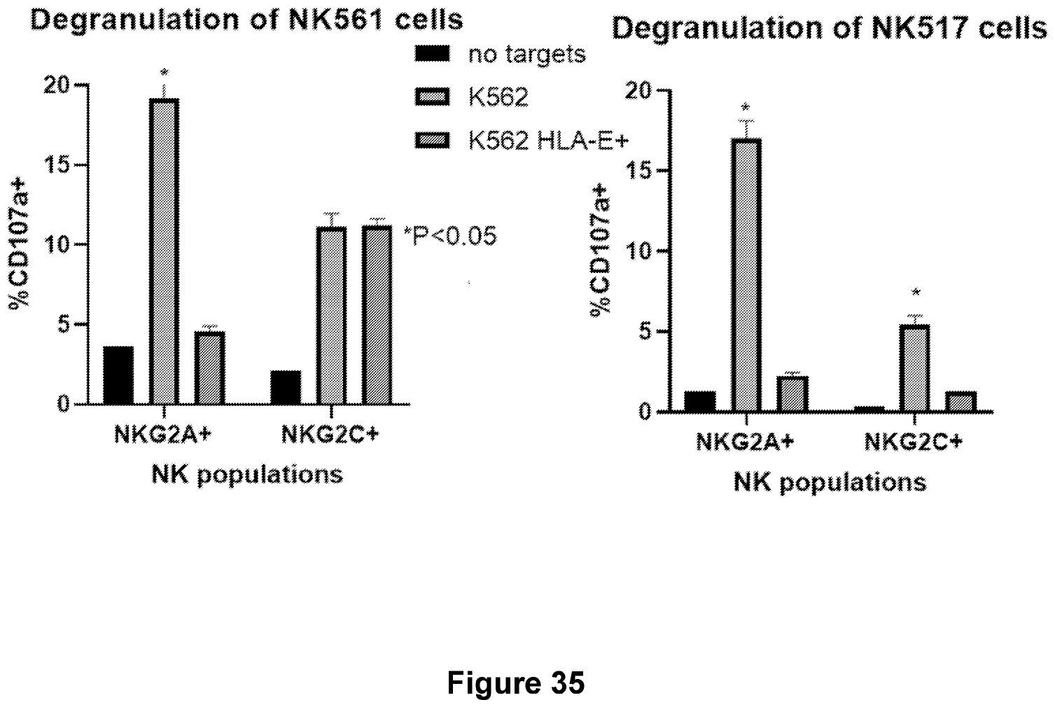

FIG. 34, comprising FIG. 34A and FIG. 34B, depicts representative results demonstrating sorted NKG2A+ NK cells were inhibited by HLA-E+ K562 cells and sorted NKG2C+ NK cells were not inhibited by HLA-E expression on K562. (**P<0.0025, ***P<0.000125; data shown at 2:1 E:T ratio) FIG. 34A depicts representative results demonstrating sorted NKG2A+ NK cells were inhibited by HLA-E+ K562 cells. FIG. 34B depicts representative results demonstrating sorted NKG2C+ NK cells were not inhibited by HLA-E expression on K562. FIG. 35 depicts representative results demonstrating that degranulation of NKG2A+ NK cells was reduced when co-cultured with HLA-E+ K562 cells. Degranulation of NKG2C+ NK cells increased when co-cultured with HLA-E+ K562 indicating NK cell activation.

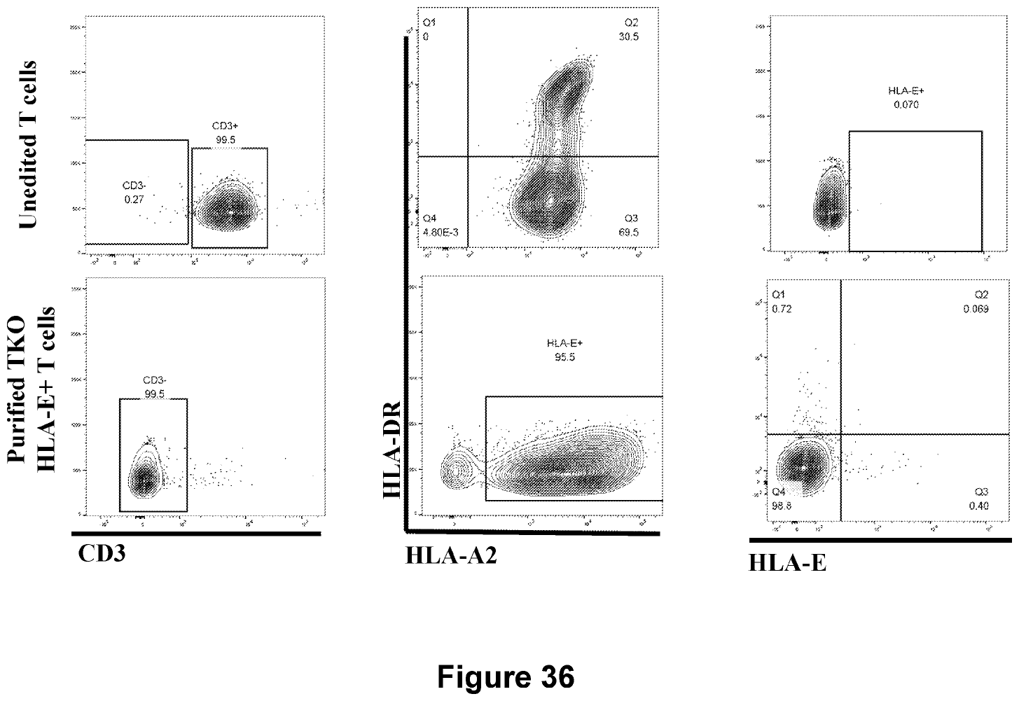

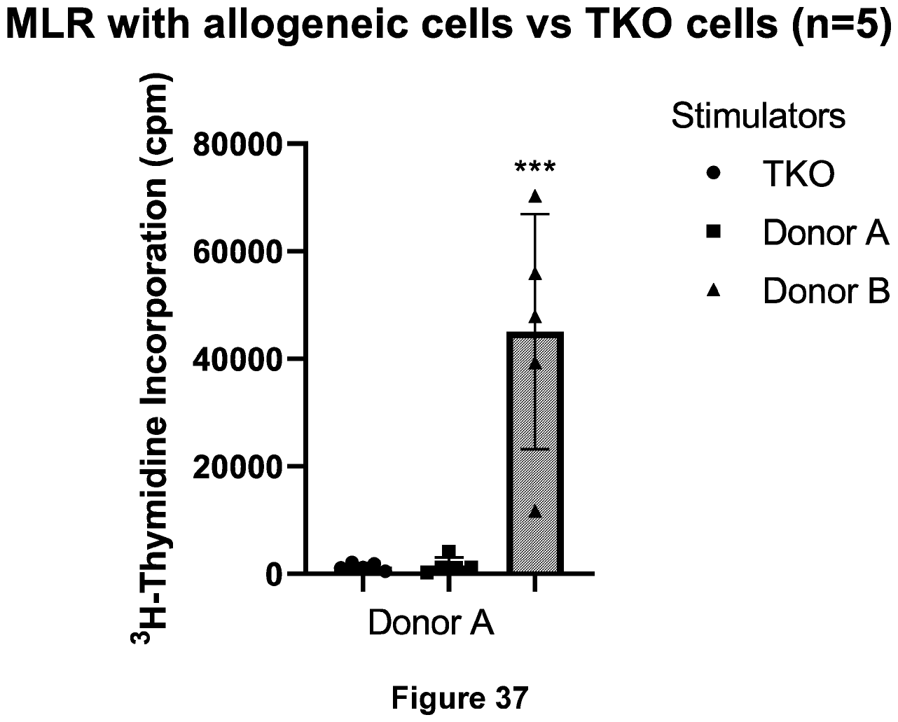

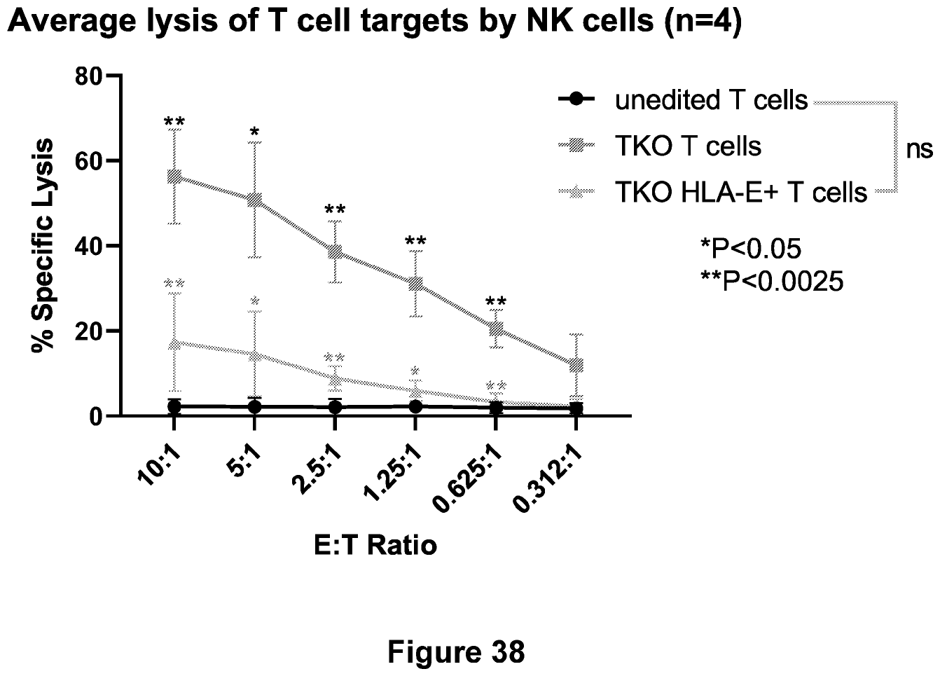

Expression of an HLA-E SCT on K562 cells consistently reduced NK cell-mediated lysis. The effect of HLA-E on NK cell activity depended on phenotypic differences of donor NK cell populations with NKG2A+ populations showing the most inhibition. HLA-E+ K562 cells persisted up to 48 hours after co-culture with NK cells. Finally, HLA-E/C complex expression on TKO T cells lead to protection against NK lytic activity (FIG. 36 through FIG. 38). More specifically, FIG. 36 depicts representative results demonstrating editing strategy and efficiency of generating TKO HLA-E+ T cells and FIG. 37 depicts representative results demonstrating TKO T cells were not recognized by allogeneic donor PBMCs as an alloreactive response was not measured in a mixed lymphocyte reaction (MLR). FIG. 38 depicts representative results demonstrating TKO CAR T cells were targeted and killed by NK cells due to “missing-self” response. HLA-E expression on TKO CAR T cells inhibited NK cell mediated-lysis.

Altogether these data demonstrate the effectiveness of selected HLA-E/peptide complexes to inhibit NK cell activation due to the “missing-self” response. Modifications such as the one described here prevent UCART cell clearance due to host recognition (NK cell activation) and may ultimately lead to a more potent, safe, and long-term persistent UCART cell therapy.

### Example 3: HLA-E SCT Expression Conferred In Vivo Protection Against NK Activity

In vivo protective effect of HLA-E SCT expression on a lymphoma cell line against human NK activity was assessed in NOG hIL-15 mice. This immunodeficient mouse model expressed human IL-15, a cytokine supportive of human NK cell engraftment. NK activity was evaluated against K562 (a lymphoma cell line devoid of MHC class I) and an engineered K562 line expressing HLA-E SCT, designated as K878. Mice were IV injected with activated and expanded human NK cells then SQ injected with K562 on the left hind flank and K878 cells on the right hind flank for a dual-flank model.

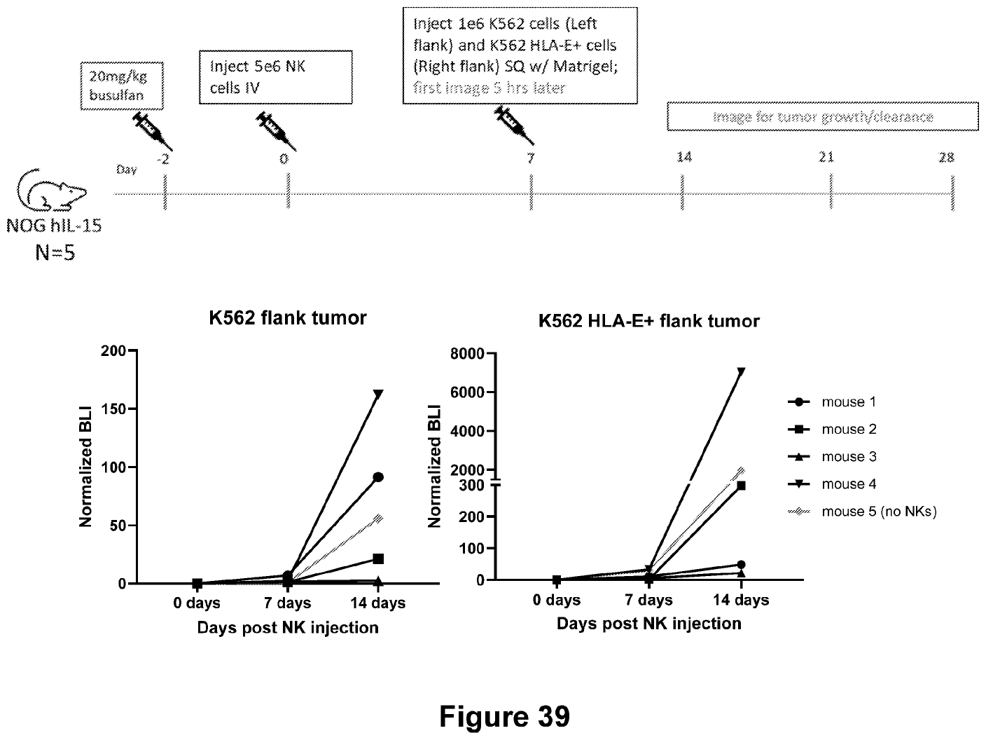

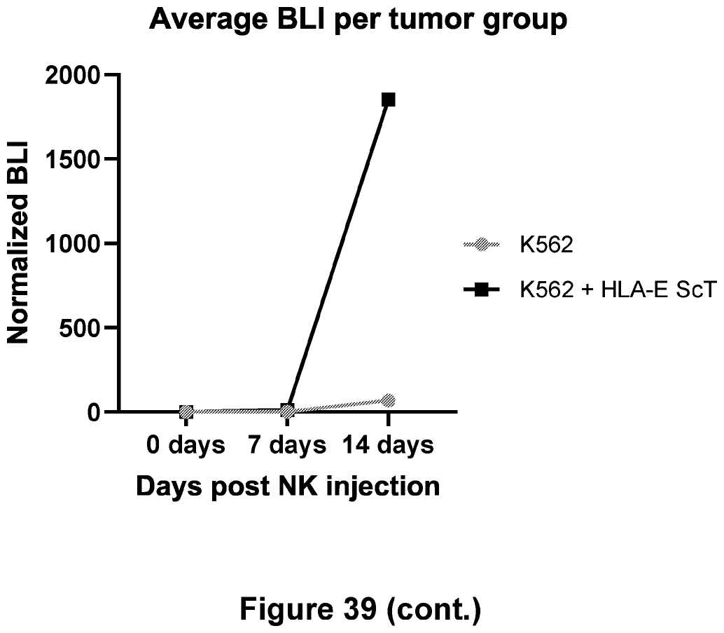

Bioluminescence imaging (BLI) once a week revealed that HLA-E SCT expression inhibited NK cells resulting in outgrowth of K878 relative to K562 by day 14 as determined by increased bioluminescence signals (FIG. 39). These results indicated that the HLA-E SCT expression conferred an in vivo protective advantage against human NK cell activity and further validating its expression in TKO T cells for the manufacturing of an allogeneic T cell therapy.

In-vivo functionality of HLA-E ScT: NOG hIL-15 mice (Taconic Biosciences) were injected intraperitoneal (IP) with 20 mg/kg busulfan two days prior to intravenous (IV) injection of 5e6 activated and expanded NK cells (FIG. 39). One week after NK injection, K562 and K562 HLA-E SCT+(K878) cells were resuspended in a 1:1 mix of PBS and Matrigel Matrix (Corning) at 1e6 cells/μl (FIG. 39). Both tumor lines expressed a click beetle luciferase marker for bioluminescence imaging (BLI) tracking. One million K562 tumor cells were injected subcutaneously (SQ) on the left hind flank of each mouse, and 1e6 K878 tumor cells were injected SQ on the right hind flank of each mouse. BLI was measured 5 hours after tumor injection (Day 0) then every week for 3 weeks (FIG. 39).

### Example 4: Sequences

HLA-A*0201 signal peptide (SEQ ID NO: 1)
VMAPRTLVL
HLA-B*0801 signal peptide (SEQ ID NO: 2)
VMAPRTVLL
HLA-C signal peptide (SEQ ID NO: 3)
VMAPRTLIL
HLA-G signal peptide (SEQ ID NO: 4)
VMAPRTLFL
Hsp60 signal peptide (SEQ ID NO: 5)
QMRPVSRVL
CMV Towne peptide (SEQ ID NO: 6)
VMAPRTLLL
CMV AF1 peptide (SEQ ID NO: 7)
VMAPRSLLL
CMV109b peptide (SEQ ID NO: 8)
VMAPRILIL
RL9-HIV peptide (SEQ ID NO: 9)
RMYSPTSIL
Mtb44 peptide (SEQ ID NO: 10)
RLPAKAPLL
K878_MA-2_005 (SEQ ID NO: 11)
TAATACGACTCACTATAGCAGCTCCCGGAGGTGCAAAA
K878_MA-1_004 (SEQ ID NO: 12)
TTCTAGCTCTAAAACTTTTGCACCTCCGGGAGCTG
consensus (SEQ ID NO: 13)
VMAPRTLL
Nucleotide sequence encoding HLA-C signal peptide (SEQ ID NO: 14)
GTGATGGCCCCAAGAACCCTGATCCTG
Nucleotide sequence encoding HLA-A*0201 signal peptide (SEQ ID NO: 15)
GTGATGGCCCCCCGGACCCTGGTGCTG
Nucleotide sequence encoding HLA-B*0801 signal peptide (SEQ ID NO: 16)
GTCATGGCGCCCCGAACCGTCCTCCTG
Nucleotide sequence encoding HLA-G signal peptide (SEQ ID NO: 17)
GTGATGGCCCCAAGAACCCTGTTCCTG
Nucleotide sequence encoding Hsp60 peptide (SEQ ID NO: 18)
CAGATGAGACCGGTGTCCAGGGTACTG
Nucleotide sequence encoding CMV Towne peptide (SEQ ID NO: 19)
GTGATGGCCCCCCGGACCCTGCTGCTG
Nucleotide sequence encoding CMV AF1 peptide (SEQ ID NO: 20)
GTGATGGCCCCCCGGAGCCTGCTGCTG
Nucleotide sequence encoding CMV109b peptide (SEQ ID NO: 21)
GTGATGGCCCCCCGGATCCTGATCCTG
Nucleotide sequence encoding RL9-HIV peptide (SEQ ID NO: 22)
CGGATGTACAGCCCCACCAGCATCCTG
Nucleotide sequence encoding Mtb44 peptide (SEQ ID NO: 23)
CGGCTGCCCGCCAAGGCCCCCCTGCTG
consensus (SEQ ID NO: 24)
GTGATGGCCCCCCGGACCCTGMT
modB2M signal peptide (SEQ ID NO: 25)
MSRSVALAVLALLSLSGLEA
Nucleotide sequence encoding modB2M signal peptide (SEQ ID NO: 26)
ATGTCACGCTCTGTCGCTCTTGCAGTACTTGCCCTGTTGAGCCTCAGCGGACTCGAAGCC
Complementarity nucleotide sequence of the nucleotide sequence encoding modB2M
signal peptide (SEQ ID NO: 27)
TACAGTGCGAGACAGCGAGAACGTCATGAACGGGACAACTCGGAGTCGCCTGAGCTTCGG
Linker 1-Amino acid sequence (SEQ ID NO: 28)
GGGASGGGGSGGGGS
Linker 1-Nucleotide sequence (SEQ ID NO: 29)
GGAGGAGGTGCGAGCGGTGGTGGAGGTAGCGGAGGTGGAGGAAGC
Linker 1-Complementarity nucleotide sequence (SEQ ID NO: 30)
CCTCCTCCACGCTCGCCACCACCTCCATCGCCTCCACCTCCTTCG
Leader-less modB2M peptide (SEQ ID NO: 31)
IQRTPKIQVYSRHPAENGKSNFLNCYVSGFHPSDIEVDLLKNGERIEKVEHSDLSFSKDWSFYLLYYTEFTPTEK
DEYACRVNHVTLSQPKIVKWDRDM
Nucleotide sequence encoding Leader-less modB2M peptide (SEQ ID NO: 32)
ATTCAAAGGACACCGAAAATCCAAGTATATAGCAGGCACCCCGCTGAAAACGGCAAAAGCAACTTTCTCAACTGT
TACGTCTCCGGCTTCCACCCCAGTGATATCGAGGTCGATCTGCTCAAGAACGGCGAACGCATCGAGAAGGTTGAA
CACAGTGATCTGTCATTTAGTAAAGATTGGTCATTTTACCTTCTCTATTATACAGAGTTTACACCAACGGAGAAG
GACGAATACGCATGTCGCGTTAATCACGTCACGCTTTCTCAACCTAAAATTGTGAAATGGGACCGTGATATG
Complementarity nucleotide sequence of the nucleotide sequence encoding Leader-less
modB2M peptide (SEQ ID NO: 33)
TAAGTTTCCTGTGGCTTTTAGGTTCATATATCGTCCGTGGGGCGACTTTTGCCGTTTTCGTTGAAAGAGTTGACA
ATGCAGAGGCCGAAGGTGGGGTCACTATAGCTCCAGCTAGACGAGTTCTTGCCGCTTGCGTAGCTCTTCCAACTT
GTGTCACTAGACAGTAAATCATTTCTAACCAGTAAAATGGAAGAGATAATATGTCTCAAATGTGGTTGCCTCTTC
CTGCTTATGCGTACAGCGCAATTAGTGCAGTGCGAAAGAGTTGGATTTTAACACTTTACCCTGGCACTATAC
Spacer-Amino acid sequence (SEQ ID NO: 34)
GGGGSGGGGSGGGGSGGGGS
Spacer-Nucleotide sequence (SEQ ID NO: 35)
GGAGGCGGTGGGTCAGGTGGAGGTGGGTCTGGCGGAGGTGGATCCGGTGGTGGAGGTAGT
Spacer-Complementarity nucleotide sequence (SEQ ID NO: 36)
CCTCCGCCACCCAGTCCACCTCCACCCAGACCGCCTCCACCTAGGCCACCACCTCCATCA
Heavy chain of HLA-E*0103 (SEQ ID NO: 37)
GSHSLKYFHTSVSRPGRGEPRFISVGYVDDTQFVRFDNDAASPRMVPRAPWMEQEGSEYWDRETRSARDTAQIFR
VNLRTLRGYYNQSEAGSHTLQWMHGCELGPDGRFLRGYEQFAYDGKDYLTLNEDLRSWTAVDTAAQISEQKSNDA
SEAEHQRAYLEDTCVEWLHKYLEKGKETLLHLEPPKTHVTHHPISDHEATLRCWALGFYPAEITLTWQQDGEGHT
QDTELVETRPAGDGTFQKWAAVVVPSGEEQRYTCHVQHEGLPEPVTLRWKPASQPTIPIVGIIAGLVLLGSVVSG
AVVAAVIWRKKSSGGKGGSYSKAEWSDSAQGSESHSL
Nucleotide sequence encoding Heavy chain of HLA-E*0103 (SEQ ID NO: 38)
GGCTCCCACTCCTTGAAGTATTTCCACACTTCCGTGTCCCGGCCCGGCCGCGGGGAGCCCCGCTTCATCTCTGTG
GGCTACGTGGACGACACCCAGTTCGTGCGCTTCGACAACGACGCCGCGAGTCCGAGGATGGTGCCGCGGGCGCCG
TGGATGGAGCAGGAGGGGTCAGAGTATTGGGACCGGGAGACACGGAGCGCCAGGGACACCGCACAGATTTTCCGA
GTGAATCTGCGGACGCTGCGCGGCTACTACAATCAGAGCGAGGCCGGGTCTCACACCCTGCAGTGGATGCATGGC
TGCGAGCTGGGGCCCGACGGGCGCTTCCTCCGCGGGTATGAACAGTTCGCCTACGACGGCAAGGATTATCTCACC
CTGAATGAGGACCTGCGCTCCTGGACCGCGGTGGACACGGCGGCTCAGATCTCCGAGCAAAAGTCAAATGATGCC
TCTGAGGCGGAGCACCAGAGAGCCTACCTGGAAGACACATGCGTGGAGTGGCTCCACAAATACCTGGAGAAGGGG
AAGGAGACGCTGCTTCACCTGGAGCCCCCAAAGACACACGTGACTCACCACCCCATCTCTGACCATGAGGCCACC
CTGAGGTGCTGGGCCCTGGGCTTCTACCCTGCGGAGATCACACTGACCTGGCAGCAGGATGGGGAGGGCCATACC
CAGGACACGGAGCTCGTGGAGACCAGGCCTGCAGGGGATGGAACCTTCCAGAAGTGGGCAGCTGTGGTGGTGCCT
TCTGGAGAGGAGCAGAGATACACGTGCCATGTGCAGCATGAGGGGCTACCCGAGCCCGTCACCCTGAGATGGAAG
CCGGCTTCCCAGCCCACCATCCCCATCGTGGGCATCATTGCTGGCCTGGTTCTCCTTGGATCTGTGGTCTCTGGA
GCTGTGGTTGCTGCTGTGATATGGAGGAAGAAGAGCTCAGGTGGAAAAGGAGGGAGCTACTCTAAGGCTGAGTGG
AGCGACAGTGCCCAGGGGTCTGAGTCTCACAGCTTGTAA
Complementarity nucleotide sequence of the sequence encoding Heavy chain of HLA-
E*0103 (SEQ ID NO: 39)
CCGAGGGTGAGGAACTTCATAAAGGTGTGAAGGCACAGGGCCGGGCCGGCGCCCCTCGGGGCGAAGTAGAGACAC
CCGATGCACCTGCTGTGGGTCAAGCACGCGAAGCTGTTGCTGCGGCGCTCAGGCTCCTACCACGGCGCCCGCGGC
ACCTACCTCGTCCTCCCCAGTCTCATAACCCTGGCCCTCTGTGCCTCGCGGTCCCTGTGGCGTGTCTAAAAGGCT
CACTTAGACGCCTGCGACGCGCCGATGATGTTAGTCTCGCTCCGGCCCAGAGTGTGGGACGTCACCTACGTACCG
ACGCTCGACCCCGGGCTGCCCGCGAAGGAGGCGCCCATACTTGTCAAGCGGATGCTGCCGTTCCTAATAGAGTGG
GACTTACTCCTGGACGCGAGGACCTGGCGCCACCTGTGCCGCCGAGTCTAGAGGCTCGTTTTCAGTTTACTACGG
AGACTCCGCCTCGTGGTCTCTCGGATGGACCTTCTGTGTACGCACCTCACCGAGGTGTTTATGGACCTCTTCCCC
TTCCTCTGCGACGAAGTGGACCTCGGGGGTTTCTGTGTGCACTGAGTGGTGGGGTAGAGACTGGTACTCCGGTGG
GACTCCACGACCCGGGACCCGAAGATGGGACGCCTCTAGTGTGACTGGACCGTCGTCCTACCCCTCCCGGTATGG
GTCCTGTGCCTCGAGCACCTCTGGTCCGGACGTCCCCTACCTTGGAAGGTCTTCACCCGTCGACACCACCACGGA
AGACCTCTCCTCGTCTCTATGTGCACGGTACACGTCGTACTCCCCGATGGGCTCGGGCAGTGGGACTCTACCTTC
GGCCGAAGGGTCGGGTGGTAGGGGTAGCACCCGTAGTAACGACCGGACCAAGAGGAACCTAGACACCAGAGACCT
CGACACCAACGACGACACTATACCTCCTTCTTCTCGAGTCCACCTTTTCCTCCCTCGATGAGATTCCGACTCACC
TCGCTGTCACGGGTCCCCAGACTCAGAGTGTCGAACATT
Representative HLA-C signal peptide-HLA-E SCT (SEQ ID NO: 40)
MSRSVALAVLALLSLSGLEAVMAPRTLILGGGASGGGGSGGGGSIQRTPKIQVYSRHPAENGKSNFLNCYVSGFH
PSDIEVDLLKNGERIEKVEHSDLSFSKDWSFYLLYYTEFTPTEKDEYACRVNHVTLSQPKIVKWDRDMGGGGSGG
GGSGGGGSGGGGSGSHSLKYFHTSVSRPGRGEPRFISVGYVDDTQFVRFDNDAASPRMVPRAPWMEQEGSEYWDR
ETRSARDTAQIFRVNLRTLRGYYNQSEAGSHTLQWMHGCELGPDGRFLRGYEQFAYDGKDYLTLNEDLRSWTAVD
TAAQISEQKSNDASEAEHQRAYLEDTCVEWLHKYLEKGKETLLHLEPPKTHVTHHPISDHEATLRCWALGFYPAE
ITLTWQQDGEGHTQDTELVETRPAGDGTFQKWAAVVVPSGEEQRYTCHVQHEGLPEPVTLRWKPASQPTIPIVGI
IAGLVLLGSVVSGAVVAAVIWRKKSSGGKGGSYSKAEWSDSAQGSESHSL
Nucleotide sequence encoding representative HLA-C signal peptide-HLA-E SCT (SEQ ID
NO: 41)
TCTAGAGCCACCATGTCACGCTCTGTCGCTCTTGCAGTACTTGCCCTGTTGAGCCTCAGCGGACTCGAAGCCGTG
ATGGCCCCAAGAACCCTGATCCTGGGAGGAGGTGCGAGCGGTGGTGGAGGTAGCGGAGGTGGAGGAAGCATTCAA
AGGACACCGAAAATCCAAGTATATAGCAGGCACCCCGCTGAAAACGGCAAAAGCAACTTTCTCAACTGTTACGTC
TCCGGCTTCCACCCCAGTGATATCGAGGTCGATCTGCTCAAGAACGGCGAACGCATCGAGAAGGTTGAACACAGT
GATCTGTCATTTAGTAAAGATTGGTCATTTTACCTTCTCTATTATACAGAGTTTACACCAACGGAGAAGGACGAA
TACGCATGTCGCGTTAATCACGTCACGCTTTCTCAACCTAAAATTGTGAAATGGGACCGTGATATGGGAGGCGGT
GGGTCAGGTGGAGGTGGGTCTGGCGGAGGTGGATCCGGTGGTGGAGGTAGTGGCTCCCACTCCTTGAAGTATTTC
CACACTTCCGTGTCCCGGCCCGGCCGCGGGGAGCCCCGCTTCATCTCTGTGGGCTACGTGGACGACACCCAGTTC
GTGCGCTTCGACAACGACGCCGCGAGTCCGAGGATGGTGCCGCGGGCGCCGTGGATGGAGCAGGAGGGGTCAGAG
TATTGGGACCGGGAGACACGGAGCGCCAGGGACACCGCACAGATTTTCCGAGTGAATCTGCGGACGCTGCGCGGC
TACTACAATCAGAGCGAGGCCGGGTCTCACACCCTGCAGTGGATGCATGGCTGCGAGCTGGGGCCCGACGGGCGC
TTCCTCCGCGGGTATGAACAGTTCGCCTACGACGGCAAGGATTATCTCACCCTGAATGAGGACCTGCGCTCCTGG
ACCGCGGTGGACACGGCGGCTCAGATCTCCGAGCAAAAGTCAAATGATGCCTCTGAGGCGGAGCACCAGAGAGCC
TACCTGGAAGACACATGCGTGGAGTGGCTCCACAAATACCTGGAGAAGGGGAAGGAGACGCTGCTTCACCTGGAG
CCCCCAAAGACACACGTGACTCACCACCCCATCTCTGACCATGAGGCCACCCTGAGGTGCTGGGCCCTGGGCTTC
TACCCTGCGGAGATCACACTGACCTGGCAGCAGGATGGGGAGGGCCATACCCAGGACACGGAGCTCGTGGAGACC
AGGCCTGCAGGGGATGGAACCTTCCAGAAGTGGGCAGCTGTGGTGGTGCCTTCTGGAGAGGAGCAGAGATACACG
TGCCATGTGCAGCATGAGGGGCTACCCGAGCCCGTCACCCTGAGATGGAAGCCGGCTTCCCAGCCCACCATCCCC
ATCGTGGGCATCATTGCTGGCCTGGTTCTCCTTGGATCTGTGGTCTCTGGAGCTGTGGTTGCTGCTGTGATATGG
AGGAAGAAGAGCTCAGGTGGAAAAGGAGGGAGCTACTCTAAGGCTGAGTGGAGCGACAGTGCCCAGGGGTCTGAG
TCTCACAGCTTGTAAGTCGAC
Complementarity nucleotide sequence of the nucleotide sequence encoding
representative HLA-C signal peptide-HLA-E SCT (SEQ ID NO: 42)
AGATCTCGGTGGTACAGTGCGAGACAGCGAGAACGTCATGAACGGGACAACTCGGAGTCGCCTGAGCTTCGGCAC
TACCGGGGTTCTTGGGACTAGGACCCTCCTCCACGCTCGCCACCACCTCCATCGCCTCCACCTCCTTCGTAAGTT
TCCTGTGGCTTTTAGGTTCATATATCGTCCGTGGGGCGACTTTTGCCGTTTTCGTTGAAAGAGTTGACAATGCAG
AGGCCGAAGGTGGGGTCACTATAGCTCCAGCTAGACGAGTTCTTGCCGCTTGCGTAGCTCTTCCAACTTGTGTCA
CTAGACAGTAAATCATTTCTAACCAGTAAAATGGAAGAGATAATATGTCTCAAATGTGGTTGCCTCTTCCTGCTT
ATGCGTACAGCGCAATTAGTGCAGTGCGAAAGAGTTGGATTTTAACACTTTACCCTGGCACTATACCCTCCGCCA
CCCAGTCCACCTCCACCCAGACCGCCTCCACCTAGGCCACCACCTCCATCACCGAGGGTGAGGAACTTCATAAAG
GTGTGAAGGCACAGGGCCGGGCCGGCGCCCCTCGGGGCGAAGTAGAGACACCCGATGCACCTGCTGTGGGTCAAG
CACGCGAAGCTGTTGCTGCGGCGCTCAGGCTCCTACCACGGCGCCCGCGGCACCTACCTCGTCCTCCCCAGTCTC
ATAACCCTGGCCCTCTGTGCCTCGCGGTCCCTGTGGCGTGTCTAAAAGGCTCACTTAGACGCCTGCGACGCGCCG
ATGATGTTAGTCTCGCTCCGGCCCAGAGTGTGGGACGTCACCTACGTACCGACGCTCGACCCCGGGCTGCCCGCG
AAGGAGGCGCCCATACTTGTCAAGCGGATGCTGCCGTTCCTAATAGAGTGGGACTTACTCCTGGACGCGAGGACC
TGGCGCCACCTGTGCCGCCGAGTCTAGAGGCTCGTTTTCAGTTTACTACGGAGACTCCGCCTCGTGGTCTCTCGG
ATGGACCTTCTGTGTACGCACCTCACCGAGGTGTTTATGGACCTCTTCCCCTTCCTCTGCGACGAAGTGGACCTC
GGGGGTTTCTGTGTGCACTGAGTGGTGGGGTAGAGACTGGTACTCCGGTGGGACTCCACGACCCGGGACCCGAAG
ATGGGACGCCTCTAGTGTGACTGGACCGTCGTCCTACCCCTCCCGGTATGGGTCCTGTGCCTCGAGCACCTCTGG
TCCGGACGTCCCCTACCTTGGAAGGTCTTCACCCGTCGACACCACCACGGAAGACCTCTCCTCGTCTCTATGTGC
ACGGTACACGTCGTACTCCCCGATGGGCTCGGGCAGTGGGACTCTACCTTCGGCCGAAGGGTCGGGTGGTAGGGG
TAGCACCCGTAGTAACGACCGGACCAAGAGGAACCTAGACACCAGAGACCTCGACACCAACGACGACACTATACC
TCCTTCTTCTCGAGTCCACCTTTTCCTCCCTCGATGAGATTCCGACTCACCTCGCTGTCACGGGTCCCCAGACTC
AGAGTGTCGAACATTCAGCTG
Complementarity nucleotide sequence of the nucleotide sequence encoding HLA-C signal
peptide (SEQ ID NO: 43)
CACTACCGGGGTTCTTGGGACTAGGAC
Heavy chain of HLA-E*0101 (SEQ ID NO: 44)
GSHSLKYFHTSVSRPGRGEPRFISVGYVDDTQFVRFDNDAASPRMVPRAPWMEQEGSEYWDRETRSARDTAQIFR
VNLRTLRGYYNQSEAGSHTLQWMHGCELGPDRRFLRGYEQFAYDGKDYLTLNEDLRSWTAVDTAAQISEQKSNDA
SEAEHQRAYLEDTCVEWLHKYLEKGKETLLHLEPPKTHVTHHPISDHEATLRCWALGFYPAEITLTWQQDGEGHT
QDTELVETRPAGDGTFQKWAAVVVPSGEEQRYTCHVQHEGLPEPVTLRWKPASQPTIPIVGIIAGLVLLGSVVSG
AVVAAVIWRKKSSGGKGGSYSKAEWSDSAQGSESHSL
Nucleotide sequence encoding Heavy chain of HLA-E*0101 (SEQ ID NO: 45)
GGCTCCCACTCCTTGAAGTATTTCCACACTTCCGTGTCCCGGCCCGGCCGCGGGGAGCCCCGCTTCATCTCTGTG
GGCTACGTGGACGACACCCAGTTCGTGCGCTTCGACAACGACGCCGCGAGTCCGAGGATGGTGCCGCGGGCGCCG
TGGATGGAGCAGGAGGGGTCAGAGTATTGGGACCGGGAGACACGGAGCGCCAGGGACACCGCACAGATTTTCCGA
GTGAACCTGCGGACGCTGCGCGGCTACTACAATCAGAGCGAGGCCGGGTCTCACACCCTGCAGTGGATGCATGGC
TGCGAGCTGGGGCCCGACAGGCGCTTCCTCCGCGGGTATGAACAGTTCGCCTACGACGGCAAGGATTATCTCACC
CTGAATGAGGACCTGCGCTCCTGGACCGCGGTGGACACGGCGGCTCAGATCTCCGAGCAAAAGTCAAATGATGCC
TCTGAGGCGGAGCACCAGAGAGCCTACCTGGAAGACACATGCGTGGAGTGGCTCCACAAATACCTGGAGAAGGGG
AAGGAGACGCTGCTTCACCTGGAGCCCCCAAAGACACACGTGACTCACCACCCCATCTCTGACCATGAGGCCACC
CTGAGGTGCTGGGCCCTGGGCTTCTACCCTGCGGAGATCACACTGACCTGGCAGCAGGATGGGGAGGGCCATACC
CAGGACACGGAGCTCGTGGAGACCAGGCCTGCAGGGGATGGAACCTTCCAGAAGTGGGCAGCTGTGGTGGTGCCT
TCTGGAGAGGAGCAGAGATACACGTGCCATGTGCAGCATGAGGGGCTACCCGAGCCCGTCACCCTGAGATGGAAG
CCGGCTTCCCAGCCCACCATCCCCATCGTGGGCATCATTGCTGGCCTGGTTCTCCTTGGATCTGTGGTCTCTGGA
GCTGTGGTTGCTGCTGTGATATGGAGGAAGAAGAGCTCAGGTGGAAAAGGAGGGAGCTACTCTAAGGCTGAGTGG
AGCGACAGTGCCCAGGGGTCTGAGTCTCACAGCTTG
Histone H2A peptide (SEQ ID NO: 46)
RIIPRHLQL
Nucleotide sequence encoding Histone H2A peptide (SEQ ID NO: 47)
CGGATCATCCCCCGGCACCTGCAGCTG

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A peptide comprising:
a human leukocyte antigen (HLA) signal peptide or a fragment thereof,
a modified beta-2-microglobulin (B2M) or a fragment thereof, and
an HLA class I histocompatibility antigen, alpha chain E (HLA-E) or a fragment thereof,
wherein the HLA signal peptide or the fragment thereof comprises at least one amino acid sequence that is at least about 70% identical to the amino acid sequence selected from SEQ ID NOs: 1-10, 13, and 46.

2. The peptide of claim 1, wherein the peptide is a single chain trimer.

3. The peptide of claim 1, wherein the HLA signal peptide comprises at least one selected from the group consisting of HLA-A*02:01 signal peptide or a fragment thereof, HLA-B*08:01 signal peptide or a fragment thereof, HLA-C*03:01 signal peptide or a fragment thereof, HLA-G signal peptide or a fragment thereof, HSP60 signal peptide or a fragment thereof, CMV Towne signal peptide or a fragment thereof, CMV AF1 signal peptide or a fragment thereof, CMV 109b signal peptide or a fragment thereof, RL9HIV signal peptide or a fragment thereof, and Mtb44 signal peptide or a fragment thereof.

4. The peptide of claim 1, wherein the HLA-E is a human HLA-E or a fragment thereof.

5. The peptide of claim 1, wherein the modified B2M comprises at least one selected from the group consisting of a modified B2M signal peptide or a fragment thereof and leader-less modified B2M or a fragment thereof.

6. The peptide of claim 5, wherein
the modified B2M signal peptide or the fragment thereof is linked to the HLA signal peptide or the fragment thereof;
the HLA signal peptide or the fragment is linked to the leader-less modified B2M or the fragment thereof, and
the leader-less modified B2M or the fragment thereof is linked to the HLA-E or the fragment thereof.

7. The peptide of claim 6, wherein the peptide further comprises at least one linker and at least one spacer.

8. The peptide of claim 7, wherein the peptide comprises an amino acid sequence that is at least about 70% identical to the amino acid sequence set forth in SEQ ID NO: 40.

9. The peptide of claim 1, wherein the peptide prevents, reduces, or inhibits a natural killer (NK) cell-mediated killing of at least one donor cell.

10. The peptide of claim 1, wherein the peptide increases the persistence or reduces the clearance of at least one donor cell.

11. A composition comprising at least one peptide of any one of claims 1-10.

12. The composition of claim 11, wherein the composition is a pharmaceutically acceptable composition.

14. A nucleic acid molecule comprising:
a nucleotide sequence encoding a human leukocyte antigen (HLA) signal peptide or a fragment thereof,
a nucleotide sequence encoding a modified beta-2-microglobulin (B2M) or a fragment thereof, and
a nucleotide sequence encoding an HLA class I histocompatibility antigen, alpha chain E (HLA-E) or a fragment thereof;
wherein the HLA signal peptide or the fragment thereof comprises at least one amino acid sequence that is at least about 70% identical to the amino acid sequence selected from SEQ ID NOs: 1-10, 13, and 46.

15. The nucleic acid molecule of claim 14, wherein the nucleotide sequence encoding the HLA signal peptide or the fragment thereof comprises at least one nucleotide sequence that is at least about 70% identical to the nucleotide sequence selected from SEQ ID NOs: 14-24 and 47.

16. The nucleic acid molecule of claim 14, wherein the nucleic acid molecule comprises a nucleotide sequence that is at least about 70% identical to the nucleotide sequence set forth in SEQ ID NO: 41.

17. A nucleic acid molecule comprising
a nucleotide sequence encoding a human leukocyte antigen (HLA) signal peptide or a fragment thereof,
a nucleotide sequence encoding a modified beta-2-microglobulin (B2M) or a fragment thereof, and
a nucleotide sequence encoding an HLA class I histocompatibility antigen, alpha chain E (HLA-E) or a fragment thereof;
wherein the nucleotide sequence encoding the HLA signal peptide or the fragment thereof comprises at least one nucleotide sequence that is at least about 70% identical to the nucleotide sequence selected from SEQ ID NOs: 14-24 and 47.

18. The nucleic acid molecule of claim 17, wherein the HLA signal peptide comprises at least one selected from the group consisting of HLA-A*02:01 signal peptide or a fragment thereof, HLA-B*08:01 signal peptide or a fragment thereof, HLA-C*03:01 signal peptide or a fragment thereof, HLA-G signal peptide or a fragment thereof, HSP60 signal peptide or a fragment thereof, CMV Towne signal peptide or a fragment thereof, CMV AF1 signal peptide or a fragment thereof, CMV 109b signal peptide or a fragment thereof, RL9HIV signal peptide or a fragment thereof, and Mtb44 signal peptide or a fragment thereof.

19. The nucleic acid molecule of claim 17, wherein the HLA-E is a human HLA-E or a fragment thereof.

20. The nucleic acid molecule of claim 17, wherein the nucleotide sequence encoding the modified B2M comprises at least one selected from the group consisting of a nucleotide sequence encoding a modified B2M signal peptide or a fragment thereof and a nucleotide sequence encoding a leader-less modified B2M or a fragment thereof.

21. The nucleic acid molecule of claim 20, wherein
the nucleotide sequence encoding the modified B2M signal peptide or the fragment thereof is linked to the nucleotide sequence encoding the HLA signal peptide or the fragment thereof,
the nucleotide sequence encoding the HLA signal peptide or the fragment is linked to the nucleotide sequence encoding the leader-less modified B2M or the fragment thereof, and
the nucleotide sequence encoding the leader-less modified B2M or the fragment thereof is linked to the nucleotide sequence encoding the HLA-E or the fragment thereof.

22. The nucleic acid molecule of claim 21, wherein the nucleic acid molecule further comprises at least one nucleotide sequence encoding a linker and at least one nucleotide sequence encoding a spacer.

23. The nucleic acid molecule of claim 22, wherein the nucleic acid molecule comprises a nucleotide sequence that is at least about 70% identical to the nucleotide sequence set forth in SEQ ID NO: 41.

24. The nucleic acid molecule of claim 17, wherein the nucleic acid molecule prevents, reduces, or inhibits a natural killer (NK) cell-mediated killing of at least one donor cell.

25. The nucleic acid molecule of claim 17, wherein the nucleic acid molecule increases the persistence or reduces the clearance of at least one donor cell.

26. A composition comprising at least one nucleic acid molecule of any one of claims 14-25.

27. A genetically engineered cell comprising at least one nucleic acid molecule of any one of claims 14-25.

28. The genetically engineered cell of claim 27, wherein the genetically engineered cell is modified to not express at least one selected from the group consisting of a beta-2-microglobulin (B2M), class II major histocompatibility complex transactivator (CIITA), and native T cell receptor (TCR).

29. The genetically engineered cell of claim 28, wherein the genetically engineered cell is a triple knockout (TKO) cell that does not express at least one selected from the group consisting of a major histocompatibility complex (MHC) I, MHC II, and native TCR.

30. The genetically engineered cell of claim 29, wherein the genetically engineered cell is selected from the group consisting of an autologous cell, allogenic cell, alloresponsive cell, T cell, induced pluripotent stem cell (IPSC), chimeric antigen receptor (CAR) cell, and any combination thereof.

31. The genetically engineered of claim 30, wherein the T cell is selected from the group consisting of an alloresponsive T cell, T cell bearing engineered TCRs, allo-specific T cell, T cell bearing alloreactive TCR, CAR T cell, engineered TCR-expressing T cell, and any combination thereof.

32. A method of preventing, reducing, or eliminating an allograft or xenograft rejection in a subject in need thereof, wherein the method comprises administering a therapeutically effective amount of at least one cell of claim 27 to the subject.

33. A method of preventing, reducing, or eliminating an alloresponse in a subject in need thereof, wherein the method comprises administering a therapeutically effective amount of at least one cell of claim 27 to the subject.

34. A method of depleting the level of an alloresponsive cell in a subject in need thereof, wherein the method comprises administering a therapeutically effective amount of at least one cell of claim 27 to the subject.

35. A method of inducing an allogeneic tolerance in a subject in need thereof, wherein the method comprises administering a therapeutically effective amount of at least one cell of claim 27 to the subject.

36. A method of improving the effectiveness of a CAR therapy in a subject in need thereof, wherein the method comprises administering a therapeutically effective amount of at least one CAR cell of claim 30 to the subject.

37. A method of improving the effectiveness of an engineered TCR cell therapy in a subject in need thereof, wherein the method comprises administering a therapeutically effective amount of at least one T cell of claim 31 to the subject.

38. The method of any one of claims 32-37, wherein the subject has at least one selected from the group consisting of an organ transplantation, tissue transplantation, cell transplantation, allotransplantation, intestinal transplantation, reconstructive transplantation, autoimmune disease or disorder, graft-versus-host disease (GvHD), disease or disorder associated with at least one HLA receptor, disease or disorder associated with at least one HLA-containing receptor, disease or disorder associated with at least one MHC receptor, disease or disorder associated with at least one MHC-containing receptor, disease or disorder associated with expression of alloresponsive cells, disease or disorder associated with organ transplantation, disease or disorder associated with tissue transplantation, disease or disorder associated with cell transplantation, disease or disorder associated with allotransplantation, disease or disorder associated with intestinal transplantation, disease or disorder associated with reconstructive transplantation, cancer, and disease or disorder associated with cancer.

39. A method of preventing or treating a disease or disorder in a subject in need thereof, wherein the method comprises administering a therapeutically effective amount of at least one cell of claim 27 to the subject.

40. The method of claim 39, wherein the method comprises depleting, reducing, or eliminating the level of at least one selected from the group consisting of an alloresponsive cell, immune cell, T cell, B cell, natural killer cell, white blood cell, myeloid cell, and plasma cell.

41. The method of claim 39, wherein the method is an engineered TCR therapy.

42. The method of claim 39, wherein the disease or disorder is selected from the group consisting of a cancer, disease or disorder associated with cancer, and any combination thereof.

43. The method of claim 39, wherein the disease or disorder is selected from the group consisting of a disease or disorder associated with expression of alloresponsive cells, disease or disorder associated with at least one HLA receptor, disease or disorder associated with at least one HLA-containing receptor, disease or disorder associated with at least one MHC receptor, disease or disorder associated with at least one MHC-containing receptor, GvHD, autoimmune disease or disorder, disease or disorder associated with organ transplantation, disease or disorder associated with tissue transplantation, disease or disorder associated with cell transplantation, disease or disorder associated with allotransplantation, disease or disorder associated with intestinal transplantation, disease or disorder associated with reconstructive transplantation, cancer, disease or disorder associated with cancer, and any combination thereof.

44. The method of claim 43, wherein the disease or disorder associated with expression of alloresponsive cell is selected from the group consisting of an allograft rejection, immune rejection, chronic allogeneic rejection, engraftment rejection, transplant rejection, inflammation, inflammation caused by ischemia/reperfusion, infection, immune response to an allograft, and any combination thereof.

45. The method of claim 39, wherein the subject had at least one selected from the group consisting of an organ transplantation, tissue transplantation, cell transplantation, allotransplantation, intestinal transplantation, and reconstructive transplantation.

46. A method of preventing or treating a cancer in a subject in need thereof, wherein the method comprises administering a therapeutically effective amount of at least one cell of claim 27 to the subject.
